(19) United States
(12) Patent Application Publication
(10) Pub. No.: US 2010/0221709 A1
(43) Pub. Date: Sep. 2, 2010

(54) METHODS FOR IDENTIFYING AGENTS THAT MODULATE APOPTOSIS IN CELLS THAT OVER-EXPRESS A BCL-2 FAMILY MEMBER PROTEIN

(75) Inventors: David M. Hockenbery, Seattle, WA (US); Michael K. Manion, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Fred Hutchinson Cancer Research Center, Seattle, WA (US)

(21) Appl. No.: 12/400,717

(22) Filed: Mar. 9, 2009

Related U.S. Application Data
(63) Continuation of application No. 11/331,652, filed on Jan. 13, 2006, now abandoned.
(60) Provisional application No. 60/644,349, filed on Jan. 14, 2005.

(51) Int. Cl. C12Q 1/68; G01N 33/574

(52) U.S. Cl. 435/6; 435/7.21

(57) ABSTRACT

The present invention provides methods and combinations of methods for identifying agents that modulate the apoptotic state of a cell by binding to the hydrophobic groove of a Bcl-2 family member anti-apoptotic protein. In certain embodiments, the methods generally comprise the use of Bcl-2 family member proteins having one or more mutations in the hydrophobic groove that, relative to a corresponding protein lacking the mutation, affect, e.g., binding of desired agents or in vitro antimycin sensitivity without substantially altering tertiary protein structure. In these embodiments, the methods comprise the identification of agents that exhibit reduced binding affinities and/or other biological activities for the mutant proteins relative to the corresponding Bcl-2 family member lacking the mutation. In other embodiments, the methods generally comprise the detection of the ability of an agent to induce increased glucose uptake or lactate production in proportion to the level of expression of an anti-apoptotic Bcl-2 family member protein.

### CROSS REFERENCES TO RELATED APPLICATIONS

The present application is a continuation of U.S. Utility application Ser. No. 11/331,652, filed Jan. 13, 2006 which claims priority to U.S. Provisional Patent Application No. 60/644,349, filed Jan. 14, 2005, the entire disclosure of which is incorporated by reference herein.

### STATEMENT AS TO RIGHTS TO INVENTIONS MADE UNDER FEDERALLY SPONSORED RESEARCH AND DEVELOPMENT

This work was supported by grants from the National Institutes of Health: Pilot Award from Cancer Center Support Grant 5P30CA015704-3 and U01 Cooperative Agreement 1U01CA91310. The U.S. government may have certain rights in the invention.

### BACKGROUND OF THE INVENTION

Members of the evolutionarily conserved Bcl-2 family are important regulators of apoptotic cell death and survival. The proteins Bcl-2, Bcl-x_{L}, Bcl-w, A1 and Mcl-1 are death antagonists while Bax, Bak, Bad, Bcl-xs, Bid, and Bik are death agonists (Kroemer et al., Nature Med 6:614-620 (1997)). Bcl-2 family member proteins are predominantly localized in the outer mitochondrial membrane, but are also found in the nuclear membrane and endoplasmic reticulum (Kroemer et al., supra).

Among Bcl-2 family member proteins, there are several conserved amino acid motifs, designated BH1 through BH4. The pro-apoptotic members of the family, Bax and Bad, contain a BH3 domain that is sufficient to induce cell death (Chittenden et al., EMBO J. 14:5589-5596 (1995); Hunter et al., J. Biol. Chem. 271:8521-8524 (1996)). Interestingly, the BH3 domain is conserved in the anti-apoptotic proteins Bcl-2 and Bcl-x_{L}. Recently, it was reported that cleavage of Bcl-x_{L} and Bcl-2 in the loop domain removes the N-terminal BH4 domain and converts Bcl-x_{L} and Bcl-2 into a potent pro-death molecule (Cheng et al., Science 278:1966-1968 (1997); Clem et al., Proc. Nat. Acad. Sci. USA 95:554-559 (1998)).

NMR structure analysis of a complex between Bcl-x_{L} and a 16 residue peptide encompassing the Bak BH3 domain demonstrated that the BH3 peptide, in an amphipathic alpha-helical configuration, binds with high affinity to the hydrophobic pocket created by the BH1, BH2 and BH3 domains of Bcl-x_{L} (Sattler et al., Science 275:983-986 (1997)). Leucine at position 1 of the BH3 domain core and aspartic acid at position 6 are believed to be critical residues for both heterodimerization and apoptosis induction. In further support of this conclusion, a number of “BH3 only” death promoters have been identified which have no similarity to Bcl-2 beyond their BH3 domain homology (Kelekar et al., Trends Cell Biol. 8:324-330 (1998)). These include Bik, Bim, Hrk, Bad, Blk, and Bid, which cannot homodimerize, but rely on binding to anti-apoptotic proteins such as Bcl-2 to induce cell death.

The exact mechanisms by which Bcl-2 prevents apoptosis remain elusive. In light of the importance of mitochondria in apoptosis and the mitochondrial location of Bcl-2, it appears that one major site where Bcl-2 interrupts apoptotic signals is at the level of mitochondria. Mitochondria play a central role in mediating apoptosis in a number of apoptotic models (Kroemer et al., Immunol. Today 18:44-51 (1997); Zamzami et al., J. Exp. Med. 183:1533-1544 (1996); Zamzami et al., J. Exp. Med. 182:367-377 (1995)). Cells induced to undergo apoptosis show an early disruption of mitochondrial transmembrane potential (ΔΨₘ) preceding other changes of apoptosis, such as nuclear fragmentation and exposure of phosphatidylserine on the outer plasma membrane. Isolated mitochondria or released mitochondrial products induce nuclear apoptosis in a cell-free reconstituted system (Liu et al., Cell 86:147-157 (1996); Newmeyer et al., Cell 79:353-364 (1994)).

It has been shown that Bcl-2 inhibits apoptosis concomitant with preventing mitochondrial permeability transition and by stabilizing ΔΨₘ (Zamzami et al., J. Exp. Med. 183:1533-1544 (1996)). In the absence of Bcl-2, apoptogenic factors, such as cytochrome c and apoptosis inducing factor (AIF), are released from mitochondria in response to apoptotic triggers (Susin et al., J. Exp. Med. 184:1331-1341 (1996); Kluck et al., Science 275:1132-1136 (1997)). This release in turn leads to sequential caspase activation and results in nuclear and membrane changes associated with apoptosis.

Bcl-2 family members display a distinct tissue-specific expression. In adult human liver, Bcl-2 expression is confined to bile duct cells (Charlotte et al., Am. J. Pathol. 144:460-465 (1994)) and is absent in both normal and malignant hepatocytes. In contrast, expression of Bcl-x_{L} RNA and protein can be detected in adult quiescent hepatocytes and increases by 4 to 5 fold during the G1 phase of regenerating hepatocytes (Tzung et al., Am. J. Pathol. 150:1985-1995 (1997)). Increased Bcl-x_{L} expression is also observed in hepatoma cell lines, such as HepG2.

Some diseases are believed to be related to the down-regulation of apoptosis in the affected cells. For example, neoplasias may result, at least in part, from an apoptosis-resistant state in which cell proliferation signals inappropriately exceed cell death signals. Furthermore, some DNA viruses, such as Epstein-Barr virus, African swine fever virus and adenovirus, parasitize the host cellular machinery to drive their own replication and at the same time modulate apoptosis to repress cell death and allow the target cell to reproduce the virus. Moreover, certain diseases, such as lymphoproliferative conditions, cancer (including drug resistant cancer), arthritis, inflammation, autoimmune diseases, and the like, may result from a down regulation of cell death signals. In such diseases, it would be desirable to promote apoptotic mechanisms.

Most currently used chemotherapeutic agents target cellular DNA and induce apoptosis in tumor cells (Fisher et al., Cell 78:539-542 (1994)). A decreased sensitivity to apoptosis induction has emerged as an important mode of drug resistance. In particular, over-expression of Bcl-2 and Bcl-x_{L} confers resistance to multiple chemotherapeutic agents, including alkylating agents, antimetabolites, topoisomerase inhibitors, microtubule inhibitors and anti-tumor antibiotics, and may constitute a mechanism of clinical chemoresistance in certain tumors (Minn et al., Blood 86:1903-1910 (1995); Decaudin et al., Cancer Res. 57:62-67 (1997)).

Neither Bcl-2 nor Bcl-x_{L}, however, protects cells from every apoptotic inducer. For example, over-expression of Bcl-2 offers little protection against Thy-1-induced thymocyte death and Fas-induced apoptosis (Hueber et al., J. Exp. Med. 179:785-796 (1994); Memon et al., J. Immunol. 15:4644-4652 (1995)). At the mitochondrial level, Bcl-2 over-expressed in the outer mitochondrial membrane inhibits PT pore induction by t-butyl-hydroperoxide, protonophore and atractyloside, but not by calcium ions, diamide or caspase 1 (Zamzami et al., J. Exp. Med. 183:1533-1544 (1996); Susin et al., J. Exp. Med. 186:25-37 (1997)). Thus, one class of mitochondrially-active agents may directly affect the mitochondrial apoptosis machinery while bypassing the site of Bcl-2 function and the protection offered by Bcl-2 family members. An agent of this type may potentially be useful in overcoming the multi-drug resistance imparted by Bcl-2 or Bcl-x_{L} and are of great need in the art.

The antimycins constitute a class of mitochondrially-active agents. The antimycins generally comprise a N-formylamino salicylate moiety linked to a dilactone ring through an amide bond. The antimycins differ in the hydrophobic R groups attached to the dilactone ring opposite the amide bond. (See, e.g., Rieske, Pharm. Ther. 11:415-420 (1980)). For example, antimycin A₁ has a hexyl group at the R₁ position (Formula I) of the dilactone ring while antimycin A₃ has a butyl group at that position.) Extensive literature has been published on the structure-activity relationship of the antimycins and their inhibition of cytochrome bc₁ (Miyoshi et al., Biochim. Biophys. Acta 1229:149-154 (1995); Tokutake et al., Biochim. Biophys. Acta 1142:262-268 (1993); Tokutake et al., Biochim. Biophys. Acta 1185:271-278 (1994)). The published structure of cytochrome bc₁ complex with bound antimycin A₁ reveals that antimycin A₁ occupies a position in the Qi ubiquinone binding site on cytochrome b (Xia et al., Proc. Nat. Acad. Sci. USA 94:11399-11404 (1997)). The antimycins generally inhibit mitochondrial respiration, which suggests that the differences in the hydrophobic R groups on the dilactone ring are not critical for cytochrome b binding. Mutagenesis and structure-activity studies of antimycin A demonstrate that the cytochrome bc₁-inhibitory activity is highly dependent on the N-formylamino salicylic acid moiety (Tokutake et al. (1994), supra). Methylation of the phenolic hydroxyl or modification of the N-formylamino group both significantly reduce the ability of antimycin A to bind to and inhibit cytochrome bc₁. Methylation of the phenolic hydroxyl diminishes inhibitory activity by 2.5 logs. Substitution of the formylamino group with acetylamino and propylamino groups at the 3-position reduce cytochrome bc₁ activity by 1.2 and 2.4 logs, respectively. Thus, the N-formylamino salicylate moiety is generally understood to be important for binding of the antimycins to cytochrome b.

Two antimycins, antimycin A₁ and A₃, have been discovered to inhibit the activity of the anti-apoptotic Bcl-2 family member proteins, Bcl-2 or Bcl-x_{L}. Thus, these molecules are potentially useful compounds for the medical profession and patients suffering from proliferative disease and other diseases where apoptosis is inappropriately regulated. The antimycins are toxic, however, because they also inhibit mitochondrial respiration. Additional studies have been carried out that have established that non-toxic derivatives of antimycin can effectively inhibit the activity of the anti-apoptotic Bcl-2 family member proteins without blocking oxidative phosphorylation. (WO 01/14365). It is clear from these studies that additional agents can be discovered which inhibit the activity of the anti-apoptotic Bcl-2 family member proteins without blocking oxidative phosphorylation.

Therefore, there is a critical need for methods of identifying additional agents that modulate apoptosis by inhibiting the activity of anti-apoptotic Bcl-2 family member proteins and that are effective in inducing apoptosis in cells where apoptosis is inappropriately regulated, including, for example, derivatives of antimycins that induce apoptosis while exhibiting reduced inhibition of mitochondrial respiration. The present invention provides such methods and additional advantages to the skilled artisan.

### SUMMARY OF THE INVENTION

The present invention provides methods and combinations of methods for identifying agents that modulate the apoptotic state of a cell by binding to the hydrophobic groove of a Bcl-2 family member anti-apoptotic protein. In certain embodiments, the methods generally comprise the use of Bcl-2 family member proteins having one or more mutations in the hydrophobic groove that, relative to a corresponding protein lacking the mutation, affect, e.g., binding of desired agents or in vitro antimycin sensitivity without substantially altering tertiary protein structure. In these embodiments, the methods comprise the identification of agents that exhibit reduced binding affinities and/or other biological activities for the mutant proteins relative to the corresponding Bcl-2 family member lacking the mutation. In other embodiments, the methods generally comprise the detection of the ability of an agent to induce increased glucose uptake or lactate production in proportion to the level of expression of an anti-apoptotic Bcl-2 family member protein.

In particular embodiments of the invention a combination of methods are used to select agents which modulate apoptosis in a cell that over-expresses a Bcl-2 family anti-apoptotic protein. This particular combination comprises selecting the agents that 1) demonstrate the ability to selectively induce apoptosis in a cell line that over-expresses the Bcl-2 family member anti-apoptosis protein as compared to a wild-type cell; 2) demonstrate the ability to inhibit pore formation in a lipid-enclosed vesicle that has on it's surface the Bcl-2 family member anti-apoptotic protein; 3) demonstrate the ability to be competitively displaced from binding to the Bcl-2 family member anti-apoptotic protein by a BH3 peptide; and 4) lack the ability to competitively displace the BH3 peptide from binding to the Bcl-2 family member anti-apoptotic protein. Agents that demonstrate each of these activities can be distinguished from agents that bind to the hydrophobic pocket of a Bcl-2 family anti-apoptotic protein but fail to modulate apoptosis.

### BRIEF DESCRIPTION OF THE DRAWINGS

FIGS. 1A and 1B depict cell viability 24 hours after treatment with staurosporine (STS) (FIG. 1A) and antimycin (FIG. 1B) toxicity in TAMH cells over-expressing Bcl-x_{L} mutants as determined by Alamar staining.

FIGS. 2A through 2C depicts the results from fluorescence anisotropy binding experiments to determine the affinity of the interaction between Bcl-x_{L}(ΔC) and AA₁ or BH3 peptide. FIG. 2A provides the fluorescence anisotropy of AA₁ (200 nM) as measured during titration with increasing concentrations of Bcl-x_{L}(ΔC) proteins. Fraction of AA₁ bound was calculated from measured anisotropies. FIG. 2B depicts the fluorescence anisotropy of FITC-labeled BH3 peptide and fraction bound was determined as in FIG. 2A. FIG. 2C depicts a competition of AA₁ (200 nM) with increasing amounts of BH3I-1 for binding to Bcl-x_{L} (3.16 μM).

FIG. 3A through 3D depict the inhibition of Bcl-x_{L}(ΔC) pore formation by AA₁ analogs and BAK BH3 peptides. FIG. 3A demonstrates that AA₁ and MeOAA₁, but not phenacyl-AA₁ (PAC-AA₁) or BH3I-1, inhibit Bcl-x_{L}(ΔC)-induced release of calcein from liposomes. Similarly, BAK wild-type BH3 peptide, but not L78A-BH3, inhibited Bch x_{L}(ΔC) pore formation. FIG. 3B demonstrates a comparison of pore formation by Bcl-x_{L}^{mu}(ΔC) and Bcl-x_{L}^{wt}(ΔC) proteins. FIG. 3C demonstrates the ability of AA₁ to inhibit pore formation by Bcl-x_{L}^{wt}(ΔC) and Bcl-x_{L}^{mu}(ΔC) proteins (1 μM). FIG. 3D demonstrates the ability of BAK BH3 peptide to inhibit pore formation by Bcl-x_{L}^{wt}(ΔC) and Bcl-x_{L}^{mu}(ΔC) proteins (1 μM). For FIGS. 3C and 3D each data point represents the cumulative calcein flux at each concentration of AA₁. Each assay was done in triplicate with standard deviations shown.

FIGS. 4A through 4D depict alignments of side chain mutations on Bcl-x_{L}(ΔC) structure (1BXL) used as docking target for AA₁. FIG. 4A provides alignments of Bcl-x_{L}(ΔC) structure in free (light grey) and BAK-BH3-bound (black) conformations. The modeled AA₁ (dark gray) is shown in place of BAK-BH3 in the binding pocket. The Cα RMSD for residues Glu-92, Phe-97, Ala-142, and Phe-146 is 1.3 Å, whereas overall Cα is 2.5 Å. FIGS. 4B through 4D provide modeling of mutations in the hydrophobic groove of 1BXL. Stars indicate clashing contacts. FIG. 4B depicts F97W which makes two moderate clashing contacts, each at 2.6 Å. FIG. 4C depicts the A142L mutation which makes an extreme clashing contact with CD1 to 08 AA₁ at 1.2 Å. FIG. 4D depicts the F146L mutation where although Phe-146 makes two van der Waals contacts with the C27 of AA₁ the F146L mutation only makes one contact from CD1 (3.4 Å).

FIGS. 5A through 5C depict antimycin A induction of apoptosis in myeloma cell lines. FIG. 5A depicts the percentage of dead cells measured as propidium iodide (PI)-positive by flow cytometry, after 24 h treatment with 20 μg/ml antimycin A (▪) or DMSO (□). FIG. 5B depicts treatment-specific cell death (treated control percent PI-positive) for RPMI-8226 cells treated with antimycin A (20 μg/ml). FIG. 5C depicts treatment-specific apoptotic cell populations measured by percent sub-G₁ DNA content, increased Annexin V cell surface staining, or reduced DiOC₆(3) dye retention, following 24 h treatment of RPMI-8226 cells with 5 (□) or 20 (▪)μg/ml antimycin A.

FIG. 6A through 6D depict a comparison of antimycin A and 2-OMe antimycin A in growth and viability assays. Myeloma cell proliferation determined by [³H]-thymidine incorporation, normalized to untreated control cells. (♦) H929, (▪) U266, () RPMI-8226 cells. FIG. 6A depicts antimycin A₃ dose response. FIG. 6B depicts the 2-OMe antimycin A₃ dose response. FIG. 6C depicts two-color analysis by flow cytometry of PI uptake and DiOC₆(3) dye retention for NCI-H929 cells treated for 18 h with 5 or 20 μg/ml antimycin A₃ (upper and lower left) or 2-OMe antimycin A₃ (upper and lower right). FIG. 6D depicts cell lines treated for 24 h with 20 μg/ml (□) 2-OMe antimycin A₃, or 10 μg/ml (▪) oligomycin (ATP synthase inhibitor) prior to PI dye exclusion assays of cell viability. Cell death is expressed as percentage of total cells.

FIG. 7A through 7C depict induction of aerobic glycolysis in TAMH cells. FIG. 7A depicts oxygen consumption by TABX2S cells. Arrows indicate successive additions of 2-OMe antimycin A₁ (10 μg) or antimycin A₁(10 μg). FIG. 7B depicts changes in lactate accumulation (□) and glucose consumption (▪) for TABX2S cells treated with 2-OMe antimycin A₁ plotted as percentage change from control cultures. Measurements for 120 s of drug exposure using μS-LOV. FIG. 7C depicts a comparison of glucose uptake responses to 2-OMe antimycin A₁ (10 μg/ml) for TAMH cells with high (2S), medium (Neo) and low Bcl-x_{L} expression (1A), measured as above. Plotted as absolute change from control cultures.

FIG. 8A through 8B depict the induction of aerobic glycosis in RPMI-8226 cells. FIG. 8A depicts mitochondrial membrane potential measured as quenching of safranin O fluorescence in digotonin-permeabilized RPMI-8226 cells. FIG. 8B depicts glucose consumption in 1×10⁶ RPMI-8226 cells treated with 1 μM 2-OMe antimycin A₁ or untreated for 24 h.

FIG. 9 depicts combined cytotoxicity of 2-OMe antimycin A and standard anticancer agents. Concentrations of standard chemotherapeutic agents were adjusted to achieve <20% cell death as single agents over 24 h treatments. Cells treated with 20 μg/ml 2-OMe antimycin A₁ and indicated concentrations of etoposide, melphalan or daunorubicin. Predicted viability at 24 h is derived from additive cytotoxicity of single agents (open symbols). Observed viability for combination (closed symbols). Each symbol represents separate experiment. Viabilities (PI-negative) are normalized to untreated cells.

FIG. 10 depicts resistance of normal bone marrow hematopoietic cells to antimycin A and 2-OMe antimycin A. Compounds were added to unfractionated human bone marrow mononuclear cells at indicated concentrations and incubated for 48 h. Lymphoid (□) and myeloid (▪) populations were gated by forward and side scatter profiles and cell death within these populations was determined by propidium iodide (PI) uptake.

FIGS. 11A through 11B depict myeloma tumor response in NOD/SCID mice. RPMI-8226 tumors were seeded by subcutaneous inoculation at day 0. FIG. 11A depicts the myeloma tumor response when 2-OMe antimycin A₁ was administered parenterally at 10 mg/ml on alternate days from day 6 to day 13 and day 26 to day 33 for a total of 6 to 7 doses (▾). Control animals received vehicle injections (♦). FIG. 11B depicts the delayed treatment of two mice with 10 mg/ml 2-OMeAA on alternate days from day 43 to day 48 (total of 3 doses).

### DESCRIPTION OF THE SPECIFIC EMBODIMENTS

The present invention provides methods for assaying candidate compounds to identify agents that modulate the activity of an anti-apoptotic Bcl-2 family member protein. In one aspect, the methods are based, inter alia, on the discovery that desired apoptosis-modulating compounds exhibit reduced binding affinities for Bcl-2 family member proteins having specific mutations in the hydrophobic groove formed by the BH1, BH2, and BH3 domains. Further, the agents also demonstrate reduced apoptosis-modulating activity in cells expressing the mutant Bcl-2 family member proteins. In certain embodiments, Bcl-2 family member proteins having one or more of these mutations are used in cell-based screening methods to identify candidate agents having the desired activity. Cell-based screens are used to determine relative levels of an apoptosis-associated physiological change (for example, cell death, cell shrinkage, chromosome condensation and migration (e.g., loss of DNA from cell nuclei or fragmentation of DNA as assessed by gel electrophoresis), and/or mitochondria swelling) induced by an agent in cells expressing a Bcl-2 family member mutant protein and cells expressing a corresponding wild-type Bcl-2 family member protein. In other embodiments, Bcl-2 family member proteins having one or more of the specific mutations are used in in vitro screening methods or, alternatively, in computer-based screening methods (e.g., virtual docking) to identify candidate agents.

Further, in another aspect, the methods are based on the discovery that desired apoptosis-modulating agents increase cellular glucose uptake and lactate production in proportion to the intracellular level of a Bcl-2 family member target protein. Cells treated with candidate agents are therefore screened for increased glucose uptake or lactate production relative to untreated cells to identify an agent that modulates the Bcl-2 family member protein activity.

In yet another aspect, the methods utilize a cell line adapted to growth in the presence of a 2-methoxy antimycin A derivative as further described herein (e.g., 2-MeO antimycin A₁ or A₃). Agents are screened for the ability to inhibit oxygen consumption in the 2-MeOAA resistant cell line, but not in the parental cell line.

To further confirm apoptosis-modulating biological activity, identified agents are tested in a combination of secondary screens. For example, agents identified in in vitro, computer-based, or glucose uptake screening methods can be further tested and screened in, e.g., a combination of cell-based screening methods as described herein for detecting apoptosis-associated physiological changes. In addition, identified agents can be tested, for example, for anti-proliferative activity in vivo (e.g., for anti-tumorigenic activity in a tumor xenograft model).

### DEFINITIONS

The term “apoptosis” refers to a regulated network of biochemical events which lead to a selective form of cell suicide, and is characterized by readily observable morphological and biochemical phenomena, such as the fragmentation of the deoxyribonucleic acid (DNA), condensation of the chromatin, which may or may not be associated with endonuclease activity, chromosome migration, margination in cell nuclei, the formation of apoptotic bodies, mitochondrial swelling, widening of the mitochondrial cristae, opening of the mitochondrial permeability transition pores and/or dissipation of the mitochondrial proton gradient.

The term “agent” is used herein to denote a chemical compound, or a mixture of chemical compounds, salts and solvates thereof, and the like, which are potentially capable of interacting with an anti-apoptotic Bcl-2 family member protein. In certain embodiments, the agent comprises an antimycin derivative or analog (e.g., a 2-methoxy antimycin derivative or analog).

The term “antimycins” refers to the antimycins A_{O(a-d)}, A₁ₐ, A_{1b}, A₂, A₃, the aniline of A₃, A₄, A₅, A₆, kitamycin A and B, urauchimycin A and B, deisovaleryl blastomycin, and dehexyl-deisovaleryloxy antimycin A. The antimycins are generally represented by the following Formula (I) and have the absolute configuration [2R, 3R, 4S, 7S, 8R]:

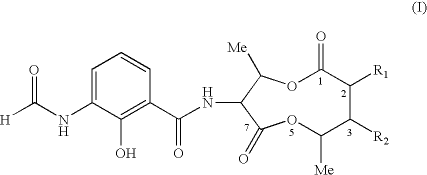

The groups at positions R₁ and R₂ vary as follows:

TABLE 1

| Name | R₁ | R₂ |
|---|---|---|
| antimycin A₀₍ₐ₎ | hexyl | hexanoic acid |
| antimycin A_{0(b)} | butyl | heptanoic acid |
| antimycin A_{0(c)} | octyl | butanoic acid |
| antimycin A_{0(d)} | heptyl | isovaleric acid |
| antimycin A_{1b} | hexyl | isovaleric acid |
| antimycin A₂ | hexyl | butanoic acid |
| antimycin A₃ | butyl | isovaleric acid |
| antimycin A₄ | butyl | butanoic acid |
| antimycin A₅ | ethyl | isovaleric acid |
| antimycin A₆ | ethyl | butanoic acid |
| kitamycin A | hexyl | hydroxyl |
| kitamycin B | isohexyl | hydroxyl |
| urauchimycin B | isohexyl | hydroxyl |
| deisovalerylblastomycin | butyl | hydroxyl |
| dehexyl-deisovalerylblastomycin | hydrogen | hydrogen |

The term “antimycin derivative or analog” refers to a chemical modification of an antimycin, by which one or more atoms of an antimycin are removed or substituted, or new atoms are added. “Antimycin derivatives or analogs” encompass both those compounds that can be made using antimycin itself as the starting molecule (e.g., isolating antimycin from a natural source and then changing the molecule) as well as compounds that are structurally related to antimycin but that are not synthesized directly from an antimycin molecule. An “antimycin derivative” further includes portions of an antimycin as well as chemical modifications thereof, and chiral variants of an antimycin. A “2-methoxy antimycin derivative or analog” (“2-OMe antimycin A derivative”) refers to an antimycin derivative or analog in which the phenolic hydroxyl group is methylated.

The term “preferentially induce” apoptosis refers to at least a 5-fold greater stimulation of apoptosis, at a given concentration of an agent, in cells that over-express a Bcl-2 family member protein as compared with cells that do not over-express the Bcl-2 family member protein (e.g., a 5-fold lower LD₅₀ or IC₅₀).

The term “substantially non-toxic” refers to an agent that induces apoptosis and/or other cellular toxicity in at least about 50 percent of cells in a cell population that over-expresses a Bcl-2 family member protein, but does not induce apoptosis and/or other cellular toxicity in more than about 5%, more preferably less than 1%, of cells in a cell population that do not over-express the Bcl-2 family member protein.

The term “Bcl-2 family member protein(s)” refers to an evolutionarily conserved family of proteins characterized by having one or more amino acid homology domains, BH1, BH2, BH3, and/or BH4. The Bcl-2 family member proteins include Bcl-2, Bcl-x_{L}, Bcl-w, A1, Mcl-1, Bax, Bak, Bad, Bcl-xs and Bid. The “Bcl-2 family member proteins” further include those proteins, or their biologically active fragments, that have at least 70%, preferably at least 80%, and more preferably at least 90% amino acid sequence identity with a Bcl-2 family member protein.

The term “anti-apoptotic Bcl-2 family member protein” refers to Bcl-2, Bcl-x_{L}, Bcl-w, A1, Mcl-1, and other proteins characterized by having one or more amino acid homology domains, BH1, BH2, BH3, and/or BH4, and that promote cell survival by attenuating or inhibiting apoptosis. The “anti-apoptotic Bcl-2 family member proteins” further include those proteins, or their biologically active fragments, that have at least 70%, preferably at least 80%, and more preferably at least 90% amino acid sequence identity with an anti-apoptotic Bcl-2 family member protein.

The terms “identity” or “percent identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using either a PILEUP or BLAST sequence comparison algorithm (see, e.g., J. Mol. Evol. 35:351-360 (1987); Higgins and Sharp, CABIOS 5:151-153 (1989); Altschul et al., J. Mol. Biol. 215:403-410 (1990); Zhang et al., Nucleic Acid Res. 26:3986-3990 (1998); Altschul et al., Nucleic Acid Res. 25:3389-3402 (1997)). Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, generally, Ausubel et al., supra).

In the context of Bcl-2 family member proteins, “correspondence” of one polypeptide sequence to another sequence (e.g., regions, fragments, nucleotide or amino acid positions, or the like) is based on the convention of numbering according to nucleotide or amino acid position number, and then aligning the sequences in a manner that maximizes the number of nucleotides or amino acids that match at each position, as determined by visual inspection or by using a sequence comparison algorithm such as, for example, PILEUP (see, e.g., supra; Higgins and Sharp, supra) or BLAST (see, e.g., Altschul et al., supra; Zhang et al., supra; Altschul et al., supra). For example, a mutant Bcl-2 family member amino acid sequence having one or more amino acid substitutions, additions, or deletions as compared to the wild-type protein may correspond to a second Bcl-2 family member amino acid sequence (e.g., the wild-type sequence or a functionally equivalent variant thereof) according to the convention for numbering the second Bcl-2 family member sequence, whereby the mutant sequence is aligned with the second Bcl-2 family member sequence such that at least 50%, typically at least 60%, more typically at least 70%, preferably at least 80%, more preferably at least 90%, and even more preferably at least 95% of the amino acids in a given sequence of at least 20 consecutive amino acids are identical. Because not all positions with a given “corresponding region” need be identical, non-matching positions within a corresponding region are herein regarded as “corresponding positions.”

As used herein, a single amino acid substitution in one (“first”) mutant Bcl-2 family member protein “corresponds” to a single amino acid substitution in a second mutant Bcl-2 family member protein (e.g., Bcl-x_{L}) where the corresponding substituted amino acid positions of the first and second mutant proteins are identical.

In the context of Bcl-2 family member protein mutants, the phrase “no substantial effect on (or ‘no substantial alteration of’) tertiary protein structure relative to the corresponding wild-type Bcl-2 family member protein” means that, when an approximation of each Cα carbon atom position in the Bcl-2 protein family member (Cα trace) of the mutant protein is superimposed onto a Cα trace of the corresponding wild-type protein and an α carbon root mean squared (RMS) difference is calculated (RMSD; i.e., the deviation of the mutant structure from that of the wild-type structure), the RMSD value is no more than about 1.0 Å, typically no more than about 0.75 Å, even more typically no more than about 0.5 Å, preferably no more than about 0.35 Å, and even more preferably no more than about 0.25 Å.

The terms “biologically active” or “biological activity” refer to the ability of a molecule or agent to modulate apoptosis, such as by binding to a Bcl-2 family member protein. Accordingly, the phrase “biologically active agent” as used herein is synonymous with the phrase “apoptosis-modulating agent.” A biologically active molecule or agent of the present invention can modulate apoptosis by causing a change in the mitochondrial proton motive force gradient (see, e.g., Example 2), by causing a change in mitochondrial swelling or the morphological characteristics of mitochondria (see, e.g., Example 2), by affecting the release of a reporter molecule, such as, for example, rhodamine 123 or calcein, from mitochondria or vesicles (see, e.g., Examples 4 and 8) comprising a pore-forming anti-apoptotic Bcl-2 family member protein (see, e.g., Example 8), or by causing any other morphological change associated with apoptosis.

The term “apoptosis-associated physiological change” refers to a change in cellular physiology that is indicative of apoptosis (e.g., cell shrinkage, chromosome condensation and migration, mitochondrial swelling, disruption of mitochondrial transmembrane potential, and the like).

The terms “therapeutically useful” and “therapeutically effective” refer to an amount of an agent that effectively modulates the apoptotic state of an individual cell, such that the inappropriately regulated cell death cycle in the cell returns to a normal state, and/or that apoptosis is induced.

The terms “diagnostically useful” and “diagnostically effective” refer to an agent (e.g., an antimycin derivative) that can be used for detecting the induction or inhibition of apoptosis in a subject. These terms further include molecules useful for detecting diseases associated with apoptosis, or the susceptibility to such diseases, and for detecting over-expression or under-expression of a Bcl-2 family member protein.

The terms “over-expression” and “under-expression” refer to increased or decreased levels of a Bcl-2 family member protein, respectively, in a cell (including, e.g., cells of tissues, organs, or populations of cells) as compared with the level of such a protein found in the same cell or a closely related non-malignant cell under normal physiological conditions.

The term “apoptosis-associated disease” includes diseases, disorders, and conditions that are linked to an increased or decreased state of apoptosis in at least some of the cells in a tissue of a subject. Such diseases include neoplastic disease (e.g., cancer and other proliferative diseases), tumor formation (see, e.g., Zornig et al., Biochim. Biophys. Acta 1551:F1-37 (2001)), arthritis (see, e.g., Liu & Pope, Curr. Opin. Pharmacol. 3:317-322 (2003)), inflammation (see, e.g., Haslett, Br. Med. Bull. 53:669-683 (1997)), autoimmune disease (see, e.g., Rathmell and Thompson, Cell 109 Suppl:S97-107 (2002); O'Reilly and Strasser, Inflamm. Res. 48:5-21 (1999)), human immunodeficiency virus (HIV) immunodeficiency syndrome (see, e.g., Kirschner et al., J. Acquir. Immune Defic. Syndr. 24:352-362 (2000)), neurodegenerative diseases (see, e.g., Honig and Rosenberg, Am. J. Med. 108:317-330 (2000)), myelodysplastic syndromes (such as aplastic anemia) (see, e.g., Greenberg, Leuk. Res. 22:1123-1136 (1998)), ischaemic syndromes (such as myocardial infarction) (see, e.g., Takemura et al., Rinsho Byori. 45:606-613 (1997)), liver diseases which are induced by toxins (such as alcohol) (see, e.g., Neuman, Rom. J. Gastroenterol. 11:3-7 (2002); Casey et al., Alcohol Clin. Exp. Res. 25(5 Suppl ISBRA):49S-53S (2001)), alopecia, damage to the skin due to UV light, lichen planus, atrophy of the skin, cataract and graft rejections.

Apoptosis-associated neurodegenerative diseases include Alzheimer's disease (see, e.g., Bamberger and Landreth, Neuroscientist 8:276-283 (2002)), Parkinson's disease (see, e.g., Lev et al., Prog. Neuropsychopharmacol. Biol. Psychiatry 27:245-250 (2003)), Huntington's disease (see, e.g., Hickey and Chesslet, Prog. Neuropsychopharmacol. Biol. Psychiatry 27:255-265 (2003)), amyotrophic lateral sclerosis and other diseases linked to degeneration of the brain, such as Creutzfeldt-Jakob disease. Apoptosis-associated diseases further include drug resistance associated with increased or decreased levels of a Bcl-2 anti-apoptotic family member protein, and also includes multiple chemotherapeutic drug resistance.

### Agents:

The screening methods according to the present invention are intended to identify agents by their biological activity mediated by binding with the hydrophobic pocket of an anti-apoptotic Bcl-2 family member protein formed by the BH1, BH2, and BH3 domains of the Bcl-2 family member protein. In particular, one or more of the disclosed methods, including methods using mutants of the Bcl-2 family member protein can be used to select suitable agents that can induce apoptosis in cells that over-express an anti-apoptotic Bcl-2 family member protein. In certain embodiments of the present invention suitable agents include molecules potentially capable of structurally interacting with Bcl-2 family member proteins through non-covalent interactions, such as, for example, through hydrogen bonds, ionic bonds, van der Waals attractions, or hydrophobic interactions. Thus, the agents will most typically include molecules with functional groups necessary for structural interactions with proteins, particularly those groups involved in hydrogen bonding. As used herein, the term “agent” is used interchangeably with the term “compound.”

Agents typically include small organic molecules such as, for example, aliphatic carbon or cyclical carbon (e.g., heterocyclic or carbocyclic structures and/or aromatic or polyaromatic structures). These structures can be substituted with one or more functional groups such as, for example, an amine, carbonyl, hydroxyl, or carboxyl group. In addition, these structures can include other substituents such as, for example, hydrocarbons (aliphatic, alicyclic, aromatic, and the like), nonhydrocarbon radicals (e.g., halo, alkoxy, acetyl, carbonyl, mercapto, sulfoxy, nitro, amide, and the like), or hetero substituents (e.g., those containing non-carbon atoms such as, for example, sulfur, oxygen, or nitrogen). In certain embodiments, the small organic molecules are antimycins or derivatives thereof.

Agents can also include biomolecules, i.e., molecules that exist in and/or can be produced by living systems as well as structures derived from such molecules. Biomolecules typically include, for example, peptides, saccharides, fatty acids, steroids, purines, pyrimidines, antimycins, and derivatives thereof. Biomolecules can include one or more functional groups such as, for example, an amine, carbonyl, hydroxyl, or carboxyl group.

In addition, agents include those synthetically or biologically produced and can include recombinantly produced structures.

In one embodiment, the agents comprise derivatives and/or analogs of an antimycin. Typically, the derivatives and/or analogs of antimycin are those antimycin molecules obtained by either synthetic, semi-synthetic means or by chemical modification of a naturally occurring antimycin molecule, such that the derivative comprises a chemical modification of the salicylate moiety and/or the dilactone moiety of an antimycin. Such derivatives can be prepared by chemically modifying an antimycin. Examples of suitable chemical modifications of a naturally occurring antimycin include addition, removal or substitution of the following substituents:
(1) hydrocarbon substituents, such as aliphatic (e.g., linear or branched alkyl, alkenyl, or alkynyl), alicyclic (e.g., cycloalkyl, or cycloalkenyl) substituents, aromatic, aliphatic and alicyclic-substituted aromatic nuclei, and the like, as well as cyclic substituents;
(2) substituted hydrocarbon substituents, such as those substituents containing nonhydrocarbon radicals which do not alter the predominantly hydrocarbon substituent; those skilled in the art will be aware of such radicals (e.g., halo (especially bromo, chloro, fluoro, or iodo), alkoxy, acetyl, carbonyl, mercapto, alkylmercapto, sulfoxy, nitro, nitroso, amino, alkyl amino, amide, and the like);
(3) hetero substituents, that is, substituents which will, while having predominantly hydrocarbyl character, contain other than carbon atoms. Suitable heteroatoms will be apparent to those of ordinary skill in the art and include, for example, sulfur, oxygen, hydroxyl, nitrogen, and such substituents as, for example, pyridyl, furanyl, thiophenyl, imidazolyl, and the like. Heteroatoms, and typically no more than one, will be present for each carbon atom in the hydrocarbon-based substituents. Alternatively, there can be no such radicals or heteroatoms in the hydrocarbon-based substituent and it will, therefore, be purely hydrocarbon.

In one embodiment, the antimycin derivative is of the following Formula (II):

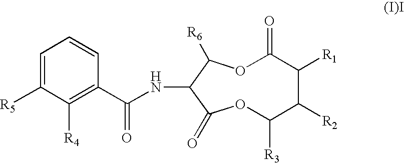

where each of positions R₁-R₆ can be independently modified. For example, each of R₁-R₃ and/or R₅ can independently be hydrogen and further R₁-R₃, and/or R₅ can independently be a C₁-C₁₀ (e.g., C₁-C₈) linear or branched alkane (e.g., methyl, ethyl, butyl, isobutyl, pentyl, isopentyl, and the like), hydroxyl, a C₁-C₁₀ (e.g., C₁-C₈) hydroxyalkane (e.g., hydroxymethyl, hydroxyethyl, hydroxypropyl, and the like), amino, an amino halogen salt (e.g., amino chloride, amino bromide or amino fluoride), a C₁-C₁₀ (e.g., C₁-C₈) di- or tri-alkylamine (e.g., methyl amine, dimethyl amine, ethyl amine, diethyl amine, and the like), a C₁-C₁₀ (e.g., C₁-C₈) amide (e.g., formylamino, acetylamino, propylamino, and the like), a C₁-C₁₀ (e.g., C₁-C₈) carboxylic acid (e.g., formic acid, acetic acid, propionic acid, butryic acid, isobutyric acid, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, ureido, guanidino, and the like). R₄ can be a methoxy group or any other group of a size that fits into the groove formed by the BH1, BH2 and BH3 domains of a Bcl-x_{L} family protein.

In another embodiment, the antimycin derivative comprises at least one of the following chemical modifications. According to Formula (II), R₁ to R₆ are typically as follows:
R₁ is hydrogen, C₁-C₁₀ (e.g., C₁-C₁₀) linear or branched alkane (e.g., methyl, ethyl, butyl, isobutyl, pentyl, isopentyl, and the like), hydroxyl, a C₁-C₁₀ (e.g., C₁-C₁₀) hydroxyalkane (e.g., hydroxymethyl, hydroxyethyl, hydroxypropyl, and the like), a C₁-C₁₀ (e.g., C₁-C₁₀) amide (e.g., N-formylamino, N-acetylamino, and the like), a C₁-C₁₀ (e.g., C₁-C₁₀) carboxylic acid (e.g., formic acid, acetic acid, propionic acid, butanoic acid, isobutanoic acids, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like);
R₂ is hydrogen, a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), a C₁-C₈ amide (e.g., N-formylamino, N-acetylamino, and the like), a C₁-C₈ carboxylic acid (e.g., formic acid, acetic acid, propionic acid, butanoic acid, isobutanoic acids, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like);
R₃ is hydrogen, a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), a C₁-C₈ amide (e.g., N-formylamino, N-acetylamino, and the like), a C₁-C₈ carboxylic acid (e.g., formic acid, acetic acid, propionic acid, butanoic acid, isobutanoic acids, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like);
R₄ is hydrogen, a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), an alkyl ether (e.g., methyl ether, ethyl ether, butyl ether, and the like) or a substituted alkyl group (e.g., an alkyl group containing an additional substituent, such as cyano, nitro, chloro, bromo, iodo, and the like);
R₅ is hydrogen, a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), amino, a C₁-C₈ di- or tri-amine (e.g., methyl amine, dimethyl amine, ethyl amine, diethyl amine, and the like), a C₁-C₈ amide (e.g., N-formylamino, N-acetylamino, and the like), a C₁-C₈ carboxylic acid (e.g., formic, acetic acid, propionic acid, butanoic acid, isobutanoic acid, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, ureido, guanidino, and the like); and
R₆ is a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), a C₁-C₈ amide (e.g., N-formylamino, N-acetylamino, and the like), a C₁-C₈ carboxylic acid (e.g., formic acid, acetic acid, propionic acid, butanoic acid, isobutanoic acids, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like);

In another embodiment, the antimycin derivative comprises at least two chemical modifications. One chemical modification reduces the affinity of the derivative for cytochrome b. The second chemical modification is in R₁-R₃ or R₆ (i.e., in the dilactone moiety).

Suitable chemical modifications, according to formula II, that decrease the affinity of the derivative for cytochrome b include, but are not limited to, one or more of the following:
R₄ is hydrogen, a C₁-C₈ linear or branched alkane (e.g., methyl, ethyl, butyl, isobutyl, pentyl, isopentyl, and the like), a C₁-C₈ hydroxyalkane (e.g., hydroxymethyl, hydroxyethyl, hydroxypropyl, and the like), an alkyl ether (e.g., methyl ether, ethyl ether, butyl ether, and the like), or a substituted alkyl group (e.g., an alkyl group containing an additional substituent, such as cyano, nitro, chloro, bromo, iodo, and the like); and
R₅ is hydrogen, a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), amino, a C₃-C₈ di- or tri-alkylamine (e.g., ethyl amine, diethyl amine, and the like), a C₁-C₈ carboxylic acid (e.g., formic, acetic acid, propionic acid, butanoic acid, isobutanoic acid, pentanoic acid, isopentanoic acids (e.g., isovaleric acid), hexanoic acid, isohexanoic acids, heptanoic acid, isoheptanoic acids, octanoic acid, isooctanoic acids, and the like), a C₂-C₈ amide (e.g., N-acetylamino, N-propylamino, N-butyrylamino, N-isobutyrylamino, N-pentanylamino, N-isopentanylamino, and the like); or a substituted alkyl group (e.g., an alkyl group containing an additional substituent, such as cyano, nitro, chloro, bromo, iodo, ureido, guanidino, and the like).

Suitable chemical modifications of the dilactone moiety include, but are not limited to, one or more of the following:
R₁ is hydrogen, C₁-C₁₀ (e.g., C₁-C₈) linear or branched alkane (e.g., methyl, ethyl, butyl, isobutyl, pentyl, isopentyl, and the like), hydroxyl, a C₁-C₁₀ (e.g., C₁-C₈) hydroxyalkane (e.g., hydroxymethyl, hydroxyethyl, hydroxypropyl, and the like), a C₁-C₁₀ (e.g., C₁-C₈) amide (e.g., N-formylamino, N-acetylamino, and the like), a C₁-C₁₀ (e.g., C₁-C₈) carboxylic acid (e.g., formic acid, acetic acid, propionic acid, butanoic acid, isobutanoic acids, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like);
R₂ is hydrogen, a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), a C₁-C₈ amide (e.g., N-formylamino, N-acetylamino, and the like), a C₁-C₈ carboxylic acid (e.g., formic acid, acetic acid, propionic acid, butanoic acid, isobutanoic acids, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like);
R₃ is hydrogen, a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), a C₁-C₈ amide (e.g., N-formylamino, N-acetylamino, and the like), a C₁-C₈ carboxylic acid (e.g., formic acid, acetic acid, propionic acid, butanoic acid, isobutanoic acids, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like); and
R₆ is a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), amino, a C₁-C₈ di- or tri-amine (e.g., methyl amine, dimethyl amine, ethyl amine, diethyl amine, and the like), or a substituted alkyl group (e.g., an alkyl group containing an additional substituent, such as cyano, nitro, chloro, bromo, iodo, and the like).

In another embodiment, the antimycin derivative is a 2-methoxy antimycin derivative of the following Formula (III):

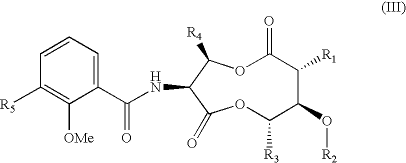

where each of positions R₁-R₅ can be independently modified, with the proviso that R₂ is an acyl group and R₅ is not acetamide, and further with the proviso that the antimycin derivative is not 2-methoxy antimycin A₃. For example, each of R₁ and R₃-R₅ can independently be hydrogen, a C₁-C₁₀ (e.g., C₁-C₁₀) linear or branched alkane (e.g., methyl, ethyl, butyl, isobutyl, pentyl, isopentyl, and the like), hydroxyl, a C₁-C₁₀ (e.g., C₁-C₁₀) hydroxyalkane (e.g., hydroxymethyl, hydroxyethyl, hydroxypropyl, and the like), amino, an amino halogen salt (e.g., amino chloride, amino bromide or amino fluoride), a C₁-C₁₀ (e.g., C₁-C₁₀) di- or tri-alkylamine (e.g., methyl amine, dimethyl amine, ethyl amine, diethyl amine, and the like), a C₁-C₁₀ (e.g., C₁-C₁₀) amide (e.g., formylamino, acetylamino, propylamino, and the like), a C₁-C₁₀ (e.g., C₁-C₁₀) carboxylic acid (e.g., formic acid, acetic acid, propionic acid, butryic acid, isobutyric acid, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, ureido, guanidino, and the like).

In certain embodiments, the 2-methoxy antimycin derivative according to Formula (III) comprises at least one of the following R groups:
R₁ is a C₁-C₁₀ linear alkane (e.g., methyl, ethyl, butyl, pentyl, hexyl, heptyl, octyl, and the like);
R₂ is either of the following Formula (IV) or Formula (V);

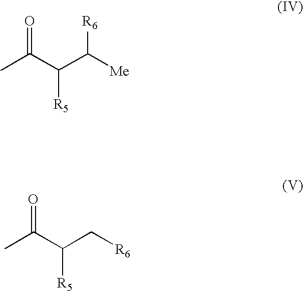

where R₅ and R₆ are each independently selected from the group consisting of a methyl group and a hydrogen;
R₃ is hydrogen, a C₁-C₈ linear or branched alkane (e.g., methyl, ethyl, butyl, isobutyl, pentyl, isopentyl, and the like), hydroxyl, a C₁-C₈ hydroxyalkane (e.g., hydroxymethyl, hydroxyethyl, hydroxypropyl, and the like), a C₁-C₈ amide (e.g., N-formylamino, N-acetylamino, and the like), a C₁-C₈ carboxylic acid (e.g., formic acid, acetic acid, propionic acid, butanoic acid, isobutanoic acids, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, and the like); and
R₄ is methyl,
   with the proviso that the antimycin derivative is not 2-methoxy antimycin A₃. In certain embodiments, the 2-methoxy antimycin derivative according to Formula (III) comprises each of R₁-R₄ as set forth above.

In yet another embodiment, the 2-methoxy antimycin derivative according to Formula (III) comprises the following Formula (Ma):

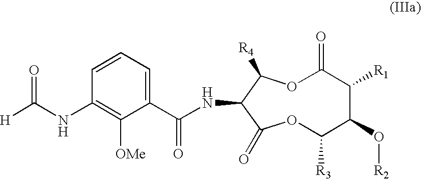

where R₁-R₄ can be independently modified as set forth above with respect to Formula (III).

In certain embodiments, the 2-methoxy antimycin derivative is of the following Formula (VI):

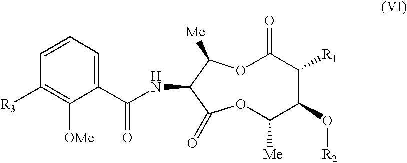

where each of positions R₁-R₃ can be independently modified, with the proviso that R₂ is an acyl group and R₃ is not acetamide, and further with the proviso that the antimycin derivative is not 2-methoxy antimycin A₃. For example, each of R₁ and R₃ can be hydrogen, a C₁-C₁₀ (e.g., C₁-C₈) linear or branched alkane (e.g., methyl, ethyl, butyl, isobutyl, pentyl, isopentyl, and the like), hydroxyl, a C₁-C₁₀ (e.g., C₁-C₈) hydroxyalkane (e.g., hydroxymethyl, hydroxyethyl, hydroxypropyl, and the like), amino, an amino halogen salt (e.g., amino chloride, amino bromide or amino fluoride), a C₁-C₁₀ (e.g., C₁-C₈) di- or tri-alkylamine (e.g., methyl amine, dimethyl amine, ethyl amine, diethyl amine, and the like), a C₁-C₁₀ (e.g., C₁-C₈) amide (e.g., formylamino, acetylamino, propylamino, and the like), a C₁-C₁₀ (e.g., C₁-C₈) carboxylic acid (e.g., formic acid, acetic acid, propionic acid, butryic acid, isobutyric acid, pentanoic acid, isopentanoic acids (e.g., isovaleric acid), hexanoic acid, isohexanoic acids, heptanoic acid, isoheptanoic acids, octanoic acid, isooctanoic acids, and the like), or a substituted alkyl group (e.g., an alkyl group containing an additional substituent, such as cyano, nitro, chloro, bromo, iodo, ureido, guanidino, and the like).

In a particular embodiment, the 2-methoxy antimycin derivative according to Formula (VI) comprises the following Formula (VIa):

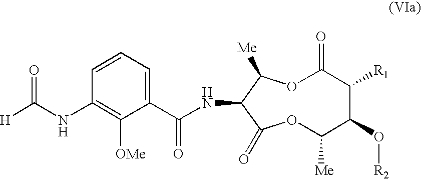

where R₁ and R₂ can be independently modified as set forth above with respect to Formula (VI).

In certain embodiments, the antimycin derivative is a lactam analogue, in which one or both lactone oxygens are replaced with nitrogen. For example, a dilactam ring can be substituted for the dilactone ring in an antimycin derivative as set forth above. In some variations, the antimycin derivative having a dilactam ring has the following Formula (VII):

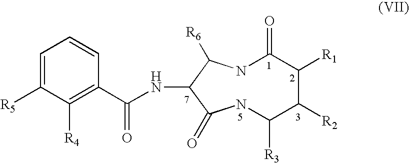

where each of R₁-R₆ can be independently modified as set forth above with respect to Formulas (II), (III), (IIIa), (VI), or (VIa). Suitable antimycin derivatives having a dilactam ring include, for example, 2-methoxy antimycin derivatives having the following Formula (VIIa):

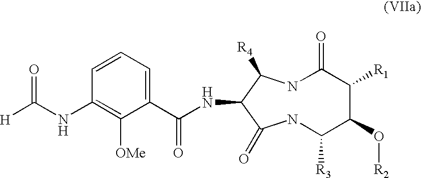

wherein each of R₁-R₄ can be independently modified as set forth above with respect to Formulas (III), (IIIa), (VI), or (VIa). Further, the ester oxygen of the lactone ring of any of the above compounds disclosed herein can be replaced with nitrogen to provide additional stability to the molecule.

Antimycin derivatives can be prepared by chemically modifying an antimycin according to standard chemical methods. Alternatively, antimycin derivatives can be prepared by de novo (“total”) chemical synthesis. See, e.g., International Patent Publication WO 01/14365.

In another embodiment, the agent is a portion of an antimycin, such as one of the functional moieties of an antimycin. Typically, such an antimycin derivative is a derivative of the dilactone moiety. Derivatives of the dilactone moiety can be prepared as further described in International Patent Publication WO 01/14365.

Libraries of agents derived from an antimycin can also be prepared by rational design. (See generally, Cho et al., Pac. Symp. Biocompat. 305-316 (1998); Sun et al., J. Comput. Aided Mol. Des. 12:597-604 (1998)). For example, libraries of antimycin derived structures can be prepared by syntheses of combinatorial chemical libraries (see generally DeWitt et al., Proc. Nat. Acad. Sci. USA 90:6909-6913 (1993); International Patent Publication WO 94/08051; Baum, Chem. Eng. News, 72:20-25 (1994); Burbaum et al., Proc. Nat. Acad. Sci. USA 92:6027-6031 (1995); Baldwin et al., Am. Chem. Soc. 117:5588-5589 (1995); Nestler et al., J. Org. Chem. 59:4723-4724 (1994); Borehardt et al., J. Am. Chem. Soc. 116:373-374 (1994); Ohlmeyer et al., Proc. Nat. Acad. Sci. USA 90:10922-10926 (1993); and Longman, Windhover's In Vivo The Business & Medicine Report 12:23-31 (1994).) Methods of making combinatorial libraries are known in the art, and include the following: U.S. Pat. Nos. 5,958,792; 5,807,683; 6,004,617; 6,077,954. The preparation of libraries suitable for use in the methods described herein are also described in International Patent Publication WO 01/14365.

### Mutant Bcl-2 Family Member Proteins:

The screening methods provided herein utilize Bcl-2 family member protein mutants. It has been discovered by Applicants that certain agents having the desired activity for modulating anti-apoptotic Bcl-2 family member protein exhibit reduced binding affinity for Bcl-2 family member proteins having one or more specific amino acid substitutions in the hydrophobic groove formed by the BH1, BH2, and BH3 domains of the protein. The desired agents further show reduced apoptosis-modulating activity in cells expressing an anti-apoptotic Bcl-2 family member protein mutants having one or more of the specific amino acid substitutions, as compared to cells expressing a corresponding wild-type Bcl-2 family member protein.

Anti-apoptotic Bcl-2 family member protein mutants suitable for use in the screening methods described herein exhibit no substantial alteration of tertiary structure relative to the corresponding wild-type Bcl-2 family member protein. In addition, in certain embodiments, the anti-apoptotic Bcl-2 family member protein mutant exhibits one or more of the following characteristics: (1) decreased binding affinity for an antimycin compound (e.g., antimycin A₁, A₂, A₃, or A₅); (2) reduced sensitivity to an antimycin compound (e.g., antimycin A₁, A₂, A₃, or A₅) when expressed in cells assayed for physiological changes associated with apoptosis; and/or (3) reduced inhibition of in vitro pore-formation activity in response to an antimycin compound. (See, e.g., antimycin binding assays, cell-based assays for apoptosis-associated changes, and pore-formation assays described herein, infra.) In an exemplary embodiment, the Bcl-2 family member protein mutant is a mutant Bcl-x_{L} protein having at least one of the following amino acid substitutions: glutamic acid (Glu, E) at position 92 is replaced by leucine (Leu, L) (E92L); phenylalanine (Phe, F) at position 97 is replaced by tryptophan (Trp, W) (F97W); leucine (Leu, L) at position 130 is replaced by alanine (Ala, A) (L130A); alanine (Ala, A) at position 142 is replaced by leucine (Leu, L) (A142L); phenylalanine (Phe, F) at position 146 is replace by leucine (Leu, L) (F146L); or tyrosine (Tyr, Y) at position 195 is replaced by glycine (Gly, G) (Y195G). In other embodiments, the anti-apoptotic Bcl-2 family member protein mutant has at least one mutation in the hydrophobic groove that corresponds to one of the above Bcl-x_{L} mutations.

The Bcl-2 family member mutant proteins can be produced by various methods known in the art. The manipulations which result in their production typically occur at the gene level. Any of various known recombinant DNA methods can be used to prepare nucleic acids encoding the desired mutant protein (see, e.g., Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999)). For example, expression cloning, genomic cloning, and PCR (see, e.g., Sambrook et al., supra; Ausubel et al., supra) can be used to obtain Bcl-2 family member nucleic acids which can be used for further manipulation. Nucleic acid sequences can also be produced by synthesis using standard methods (e.g., by use of a commercially available automated DNA synthesizer) (typically for shorter nucleic acids). The nucleic acids can then be further manipulated as desired using routine techniques. (See, e.g., Sambrook et al.; Ausubel et al., supra.) For example, Bcl-2 family member nucleic acids can be modified to prepare sequences encoding the desired Bcl-2 mutant protein using, e.g., standard in vitro site-directed mutagenesis (see, e.g., Hutchinson et al., J. Biol. Chem. 253:6551-6560 (1978)), the use of TAB® linkers (Pharmacia), PCR mutagenesis, and the like). Truncated Bcl-2 family member proteins and mutant proteins can also be produced by any of the disclosed methods and other methods well known to the skilled artisan. The truncated protein can comprise, for example, a protein wherein amino acid residues that comprise the membrane anchor region have been deleted.

Once obtained, a nucleic acid encoding the desired Bcl-2 family member protein can be inserted into an appropriate expression vector (i.e., a vector that contains the necessary elements for the transcription and translation of the inserted sequence). A variety of known host-vector systems can be utilized to express the Bcl-2 mutant protein. (See, e.g., Sambrook et al., supra; Ausubel et al., supra.) Once a suitable expression vector-host system and growth conditions are established, methods that are known in the art can be used to propagate it. The expression vector-host system can be selected, for example, for use in the cell-based evaluation of candidate agents (e.g., antimycin derivatives) according to the screening methods described herein (infra). Alternatively, the expression vector-host system can be selected for the efficient production of protein for subsequent isolation and purification of the expressed protein by standard methods such as, for example, chromatography (e.g., ion exchange, affinity, sizing column chromatography, high pressure liquid chromatography), centrifugation, differential solubility, or by any other standard technique for the purification of proteins. The purified Bcl-2 proteins can then be used to evaluate candidate agents for protein binding using, for example, the binding assays described herein (infra).

Bcl-2 family member proteins having mutations in the hydrophobic groove and which are suitable for use in the screening methods provided herein can be identified by evaluating their functional properties. Typically, suitable mutant proteins exhibit (1) no substantial alteration of tertiary structure relative to the corresponding wild-type Bcl-2 family member protein while (2) displaying decreased antimycin sensitivity in in vivo cell-based assays and/or decreased in vitro antimycin binding. (See, e.g., Table 2, which summarizes the characterization of suitable Bcl-x_{L} mutants, F97W, L130A, A142L, F146L, and Y195G; and Table 3, which summarizes Cα trace overlays of Bcl-x_{L}, point mutant structures with wild-type Bcl-x_{L}) In certain embodiments, suitable mutant proteins also exhibit reduced inhibition of pore-forming activity in response to antimycin. Also, suitable mutant proteins preferably exhibit wild-type levels of resistance to staurosporine (STS) in in vivo cell-based assays. (See, e.g., id.) The functional properties of the mutant Bcl-2 family member proteins can be evaluated using any suitable assay as described herein or otherwise known to the skilled artisan, including, for example, antimycin binding assays, cell-based assays for physiological changes associated with apoptosis, or pore-formation assays using membrane-enclosed vesicles.

TABLE 2
Summary of Characterization of Bcl-x_{L} Mutants

| | Predicted | | | | | |
|---|---|---|---|---|---|---|
| | Effect on | In vivo | In vivo | In vitro | | Major |
| | Antimycin | STS | Antimycin | Antimycin | In vitro Pore | Structural |
| Mutation | Interaction | Sensitivity | Sensitivity | Binding | Inhibition | Effects |
| WT | Binds in | − | + | + | + | *** |
| | BH3 pocket | | | | | |
| E92L | None | − | + | + | + | No |
| F97W | Clashing | − | +/− | − | − | No |
| | Contact | | | | | |
| L130A | Loss of | − | − | nd | nd | nd |
| | Contact | | | | | |
| A142L | Loss of | − | − | − | − | nd |
| | Contact | | | | | |
| F146L | Loss of | − | − | +/− | + | No |
| | Contact | | | | | |
| F146W | Clashing | + | +/− | +/− | + | Yes |
| | Contact | | | | | |
| Y195G | Loss of H- | − | − | nd | nd | nd |
| | bonding | | | | | |
| A200L | None | − | + | nd | nd | No |

TABLE 3
Summary of C α trace overlays of Bcl-x_{L} point mutants with wild-type
Bcl-x_{L}.

| | Resolution | R, R_{free} (%) | RMSD(C α) | Major Effect |
|---|---|---|---|---|
| WT | 1.9 Å | 23.8, 24.6 | — | — |
| E92L | 2.1 Å | 23.8, 25.6 | 0.21 Å | None |
| F97W | 2.7 Å | 19.7, 24.0 | 0.34 Å | None |
| F146L | 2.1 Å | 25.1, 27.2 | 0.25 Å | None |
| F146W | 2.2 Å | 25.4, 28.6 | 1.23/3.67 | Shift of α 3 |
| A200L | 2.2 Å | 25.3, 27.6 | 0.21 Å | None |

### Screening Methods for Identifying Agents

The screening methods of the present invention identify agents that modulate apoptosis by binding to the hydrophobic groove of Bcl-2 family member proteins. In one embodiment, the method generally comprises the following steps: (1) contacting a candidate compound independently with each of (a) a cell that over-expresses an anti-apoptotic Bcl-2 family member protein and (b) another cell that over-expresses a mutant Bcl-2 family member protein corresponding to the anti-apoptotic Bcl-2 family member protein, where the mutant protein has one or more mutations in the hydrophobic groove that, relative to the anti-apoptotic Bcl-2 family member protein, has no substantial effect on the tertiary protein structure while reducing the binding affinity of the protein for an antimycin compound; (2) determining whether the candidate compound modulates the activity of the anti-apoptotic Bcl-2 family member protein to produce an apoptosis-associated physiological change in the cell over-expressing the anti-apoptotic Bcl-2 family member protein; and (3) determining whether the candidate compound produces a reduced apoptosis-associated physiological change in the cell over-expressing the mutant Bcl-2 family member protein. In certain embodiments, the method further includes the steps of (4) contacting the candidate compound with a control cell that does not over-express either the anti-apoptotic Bcl-2 family member protein or the corresponding mutant and (5) determining whether the candidate compound does not substantially produce the apoptosis-associated physiological change in the control cell.

Apoptosis-associated physiological changes are indicative of binding of the candidate compound to the Bcl-2 family member protein (e.g., in the hydrophobic pocket) and can include an affect on cell death (e.g., determined by, for example, trypan blue dye exclusion), cell shrinkage, chromosome condensation and migration, mitochondria swelling, and/or disruption of mitochondrial transmembrane potential (i.e., the mitochondrial proton gradient).

The mutant Bcl-2 family member proteins suitable for use in the screening methods described herein include the mutant proteins described supra. For example, the corresponding Bcl-2 family member mutants can include (a) those Bcl-2 family member proteins having a mutation in the hydrophobic groove that, relative to the anti-apoptotic Bcl-2 family member protein, has no substantial effect on tertiary protein structure while having a reduced binding affinity for an antimycin; (b) those Bcl-2 family member proteins having a mutation that corresponds to a Bcl-x_{L} mutation that is E92L, F97W, L130A, A142L, F146L, or Y195G; and (c) a Bcl-x_{L} mutant protein having a mutation that is F97W, L130A, A142L, F146L, or Y195G.

In a particular embodiment, a candidate compound is contacted with mammalian tissue culture cells over-expressing an anti-apoptotic Bcl-2 family member protein, contacted with cells over-expressing a corresponding mutant Bcl-2 family member protein, and contacted with control cells to which no compound is added. Methods of expressing various Bcl-2 family member proteins in tissue culture cells are well known in the art. (See, e.g., Example 1, U.S. Pat. No. 5,998,583.) At various time points after contacting the candidate compound with the cells (e.g., at 6 and 24 hours), the cells from each group are trypsinized, and cell viability is determined by trypan blue dye exclusion. The number of viable cells are counted and normalized to the control group (i.e., % control=number of viable cells (treated group)/number of viable cells (control group)×100). A desired agent capable of modulating apoptosis of a cell by binding to the hydrophobic groove preferentially induces apoptosis in cells that over-express the Bcl-2 family member protein, but exhibits reduced induction of apoptosis in cells that over-express the corresponding mutant Bcl-2 family member protein (relative to cells over-expressing the anti-apoptotic Bcl-2 family member protein).

In another specific embodiment, a candidate compound is added to a) mammalian tissue culture cells over-expressing an apoptotic Bcl-2 family member protein, b) to cells over-expressing a corresponding mutant Bcl-2 family member protein, c) to cells having normal levels of the anti-apoptotic Bcl-2 family member protein, and d) to control cells to which no compound is added. At various time points after administration of the candidate compound (e.g., at 6 and 24 hours), the cells from each group are trypsinized, and cell viability is determined by trypan blue dye exclusion. The number of viable cells are counted and normalized to the control group (i.e.,% control=number of viable cells (treated group)/number of viable cells (control group)×100). A desired agent for modulating apoptosis of a cell by binding to the hydrophobic groove preferentially induces apoptosis in cells that over-express the Bcl-2 family member protein, but not cells having normal levels of the Bcl-2 family member protein, and further exhibits reduced induction of apoptosis in cells that over-express the corresponding mutant Bcl-2 family member protein (relative to cells over-expressing the anti-apoptotic Bcl-2 family member protein).

In yet another specific embodiment, the candidate compound is added a) to mammalian tissue culture cells over-expressing an anti-apoptotic Bcl-2 family member protein, b) to cells over-expressing a corresponding mutant Bcl-2 family member protein, and c) to control cells to which no compound is added. Optionally, the candidate compound is added to cells having normal levels of the anti-apoptotic family member protein. At various time points after administration of the candidate compound (e.g., at 6 and 24 hours), nuclear morphology is determined by a nucleic acid stain, such as for example, 4′-6-diamidino-2-phenylindole (DAPI). Cells in which apoptosis has occurred will exhibit characteristic changes in nuclear morphology, such as chromosome condensation and migration. Methods to monitor other physiological changes are disclosed in the Examples (infra).

In another embodiment, reagents and assay conditions which are useful for interrogating agents for utility in the present invention comprise: (1) cells which over-express an anti-apoptotic Bcl-2 family member (e.g., Bcl-2, Bcl-x_{L}, Bcl-w, A1, Mcl-1, and the like), (2) aqueous components which produce binding conditions, e.g., physiological buffers, (3) a reporter system, e.g., a cell, or a reporter molecule, and (4) a candidate compound being tested. The candidate compound can also be screened for toxicity to cells that do not over-express the anti-apoptotic Bcl-2 family member protein.

In certain embodiments, candidate compounds are initially screened for modulation of activity of cells that over-express the anti-apoptotic Bcl-2 family member protein. In one particular embodiment, a candidate compound is identified by its ability to preferentially induce apoptosis in cells transformed with a gene that encodes at least the Bcl-x_{L} BH3 binding pocket, but the compound has a reduced ability to induce apoptosis in cells over-expressing a corresponding mutant protein as described supra. The candidate compound is optionally tested for the absence of, or reduced induction of, apoptosis in a cell that does not over-express the anti-apoptotic Bcl-2 family member protein (e.g., one that has not been so transformed, or that is transformed with a control vector, or an anti-sense vector). In a particular embodiment, candidate compounds are assayed for their ability to preferentially induce apoptosis in a murine tumorigenic liver cell line which over-expresses the Bcl-x_{L} protein.

In another embodiment, the screening method includes determining the ability of a candidate compound to preferentially inhibit pore-forming activity by an anti-apoptotic Bcl-2 family member protein. Typically, a candidate compound that inhibits pore formation in this assay can be assayed in other methods provided herein. But, in an additional embodiment the activity of the compound can also be compared to a corresponding mutant Bcl-2 family member protein. This assay comprises a membrane enclosed vesicle, the vesicle having on its surface a Bcl-2 family member protein, such as Bcl-x_{L}, or Bcl-2 or the corresponding mutant protein. A reporter present within the vesicle acts as an indicator of the modulation of pore formation by the candidate compound. Suitable reporters include fluorescers, chemiluminescers, radiolabels, enzymes, enzyme cofactors, and the like.

One specific example of this assay comprises preparing large unilamellar vesicles (LUV's) containing a fluorescent reporter molecule. In a particular embodiment, LUV's (e.g., comprising 60% dioleophosphatidylcholine and 40% dioleoylphosphatidyl-glycerol) contain the fluorescent reporter calcein. When an anti-apoptotic Bcl-2 family member protein is inserted into the vesicle, the fluorescent reporter leaks out of the vesicle. Binding of a successful candidate compound being tested to the anti-apoptotic Bcl-2 family member protein disrupts pore formation, and leakage of the reporter from the vesicle is blocked. Further, successful candidate compounds typically exhibit a reduced ability to inhibit pore-forming activity of the corresponding mutant protein as compared to the anti-apoptotic Bcl-2 family member protein.

In yet another assay system, agents are identified by their ability, under binding conditions, to preferentially bind to the BH3 binding domain of an anti-apoptotic Bcl-2 family member protein (e.g., Bcl-2 or Bcl-x_{L} polypeptide) as compared to a corresponding mutant Bcl-2 family member protein. For example, one method utilizing this approach that may be pursued in the identification of such candidate compounds includes the attachment of a compound to a solid matrix, such as, e.g., agarose or plastic beads, microtiter wells, a petri dish, or a membrane composed of, for example, nylon or nitrocellulose, and the subsequent incubation of the attached compound in the presence of, independently, an anti-apoptotic Bcl-2 family member protein and a corresponding mutant Bcl-2 family member protein. Attachment to the solid support can be direct or by means of a compound-specific antibody bound directly to the solid support. After incubation, unbound protein is washed away, and bound protein is detected by methods known in the art such as, e.g., use of a labeled Bcl-2 family member-specific antibody. Other methods for determining compound binding are known in the art and include, for example, fluorescence polarization, isothermal titration calorimetry, surface plasmon resonance, NMR spectroscopy, and the like.

In yet other embodiments of the present invention, agents that modulate apoptosis by binding to the hydrophobic groove of a Bcl-2 family member protein are identified using a computer-based method. A “molecular docking” computer algorithm is used to independently score a candidate agent for binding to each of (1) the hydrophobic pocket of the Bcl-2 family member protein formed by the BH1, BH2, and BH3 domains and (2) the hydrophobic pocket of a corresponding mutant Bcl-2 family member protein as described supra. The two molecular docking scores for the candidate agent (i.e., for binding to each of the anti-apoptotic Bcl-2 family member protein and the corresponding mutant protein) are compared. A successful candidate agent exhibits a docking score for binding to the corresponding mutant protein that is significantly less than that for binding to the anti-apoptotic Bcl-2 family member protein.

Computer-based techniques for examining potential ligands (e.g., candidate agents) for binding to target molecules are well-known in the art. (See, e.g., Kuntz et al., J. Mol. Biol. 161:269-288 (1982); Kuntz, Science 257:1078-1082 (1992); Ewing and Kuntz, J. Comput. Chem. 18:1175-1189 (1997)). For example, the DOCK suite of programs is designed to find possible orientations of a ligand in a receptor site. (See, e.g., Ewing and Kuntz, supra). The orientation of a ligand is evaluated with a shape scoring function (an empirical function resembling the van der Waals attractive energy) and/or a function approximating the ligand-receptor binding energy (which is taken to be approximately the sum of the van der Waals and electrostatic interaction energies). After an initial orientation and scoring evaluation, a grid-based rigid body minimization is carried out for the ligand to locate the nearest local energy minimum within the receptor binding site. The position and conformation of each docked molecule can be optimized using the single anchor search and torsion minimization method of, for example, DOCK4.0. (See, e.g., Ewing and Kuntz, supra; Kuntz, supra).

In some embodiments of the computer-based screening methods, the structure of the Bcl-2 family member protein used for docking is derived from the Bcl-2 family member protein complexed with a polypeptide having a BH3 domain (e.g., a BH3 peptide). Alternatively, in other embodiments, the structure of unbound Bcl-2 family member protein, which typically has shallow, narrow hydrophobic groove, is used for docking. In one particular embodiment of the present invention, an agent which modulates apoptosis of a cell by binding to the hydrophobic pocket of the Bcl-2 family member protein formed by the BH1, BH2, and BH3 domains of the protein is identified by first utilizing a molecular docking algorithm to score a candidate compound for binding to the hydrophobic pocket of the Bcl-2 family member protein as determined by protein structure determination for an unliganded protein, e.g., the structure of the hydrophobic pocket without a ligand bound in to pocket. The molecular docking algorithm is then used to score the candidate compound for binding to the hydrophobic pocket of the Bcl-2 family member protein as determined by protein structure determination for the Bcl-2 family member protein bound to a ligand, such as the BH3 peptide, subsequent to subtracting the ligand coordinates; and then determining whether the docking score based on minimal docked conformation energies for the Bcl-2 family member protein hydrophobic pocket in the confirmation without the ligand is lower than the docking score for the Bcl-2 family member protein with the hydrophobic pocket in the confirmation if the ligand were bound to identify the agent. This method can identify certain agents of the present invention because ligand binding to the hydrophobic pocket of the Bcl-2 family member protein remodels the hydrophobic pocket, or groove, from the unbound structure (a closed confirmation), with widening and straightening of the cleft, Virtual screening strategies for known small molecule inhibitors of Bcl-2 family member protein, e.g., Bcl-2 or Bcl-x_{L}, were based on computational modeling of ligand docking to the open conformation of the peptide-bound groove. Using the docking simulations of this embodiment, for example using the docking algorithm of AUTODOCK 3.05, predict lower docking energy conformations for 2-MeAA₁ with the unliganded Bcl-x_{L} structure than the peptide-bound structure.

In certain embodiments, a database of agents is screened using the virtual docking techniques described herein to identify potential biologically active agents. For example, a database that represents a library of known structures (e.g., a combinatorial library; see description of combinatorial libraries, supra) can be constructed de novo using known methods for chemical database construction. Alternatively, for example, existing agent or compound databases are available for virtual screening. Known databases for virtual screening include, e.g., the MDL Available Chemicals Directory and the MDL Screening Compounds Directory (MDL Information Systems, San Leandro, Calif.); the Specs research compound database (Specs, Rijswijk, The Netherlands); and the China Natural Product Database (CNPD) (Neotrident Technology, Ltd, Kowloon, Hong Kong). Initial database mining can be performed to identify compounds having, for example, suitable characteristics for in vivo use (e.g., suitable solubility and permeability profiles) (using, for example, UC_Select, see, e.g., Skillman, 2000 Ph.D. thesis, University of California, San Francisco), followed by, e.g., visual inspection and removal of unreactive molecules.

In typical embodiments, heuristic docking and consensus scoring strategies are used in the virtual screening methods of the present invention (i.e., different docking and scoring methods are applied to evaluate the screening results). For example, following a primary screening using, e.g., DOCK4.0 (supra), top-scoring compounds can be re-scored using other docking algorithms such as, for example, GOLD, FlexX, PM (see Muegge and Martin, J. Med. Chem. 42:791-804 (1999), and/or AutoDock3.0 (see Morris et al., J. Comput. Chem. 19:1639-1662 (1998)). Optionally, following a primary and any subsequent screen(s) using individual docking algorithms, a consensus score (Cscore) can be determined by combining results from any of the individual docking programs used to score the candidate compounds (see Clark et al., J. Mol. Graph. Model 20:281-295 (2002)). Based on the scoring results from a secondary or other subsequent screen, a subset, for example, of the top-scoring molecules from the primary screen can be selected for further analyses (e.g., a tertiary virtual screen or, alternatively or additionally, biological screening assays such as, for example, any of the assays described herein or otherwise known in the art). Typically, 1 to 10% of the top scoring compounds are selected to recover all or most of the compounds of interest for secondary screening. (Waszkowycz et al., IBM Systems J. 40:360 (2001)).

In another embodiment of the present invention, biologically active agents are identified by evaluating the ability of the agents to modulate glucose uptake and/or lactate production in cells expressing a Bcl-2 family member protein. It has been discovered by Applicants that a desired apoptosis-modulating agent such as, for example, an antimycin derivative (e.g., 2-methoxy antimycin derivatives) increases cellular glucose uptake or lactate production in proportion to the level of expression of a Bcl-2 family member target protein.

Generally, the method includes the following steps: (1) administering a candidate compound independently to each of two cells, or populations of cells, expressing a Bcl-2 family member protein, where one cell, or cell population, has a higher level of expression of the Bcl-2 family member protein relative to the other cell, or cell population; (2) determining in each cell the level of glucose uptake or lactate production; and (3) determining whether the cell having higher expression of the Bcl-2 family member protein has a higher level of glucose uptake or lactate production relative to the cell having lower expression of the Bcl-2 family member protein.

Methods for assaying glucose production or lactate production are well-known in the art. (See, e.g., Schultz and Ruzicka, Analyst 127:1293-1298 (2002); Schultz et al., Analyst 127:1583-1588 (2002).) For example, glucose and lactate concentrations can be assayed as substrates in first-order NAD-linked enzymatic reactions, with NADH generation monitored by absorbance at 340 nm. The glucose reagent can include final concentrations of, e.g., >1500 U/l hexokinase, >3200 U/l glucose-6-phosphate dehydrogenase, 2.1 mM ATP, and 2.5 mM NAD⁺. The lactate reagent can include final concentrations of, e.g., 2000 U/ml LDH and 2.5 mM NAD⁺ in glycine buffer. For experiments, cells are typically maintained in an appropriate buffer (e.g., Hanks balanced salt solution (HBSS)). The candidate compound is administered to the cells, followed by an incubation period to allow depletion of glucose and accumulation of lactate. A fixed volume of tissue culture supernatant (e.g., buffer solution incubated in the presence of treated cells) is then added to glucose or lactate reagent and 340 nm absorbance is recorded.

Further, glucose uptake and lactate production can be analyzed by, for example, sequential injection analysis of cells attached to beads. (See, e.g., Schultz and Ruzicka, supra; Schultz et al., supra.) For example, cells can be attached to, e.g., Cytopore™ beads (Amersham Pharmacia Biotech, Upsala, Sweden; hydrated in Hanks balanced salt solution (HBSS; Gibco-BRL, Grand Island, N.Y.); autoclaved according to the manufacturer's instructions; and incubated in, e.g., serum-containing media). Typically, cells are transferred to the bead slurry (at a ratio of, e.g., 50 cells per bead) and grown in spinner culture flasks with gentle stirring, followed by collection of the cell-coated beads for metabolic studies at cell densities of for example, about 100 to about 500 cells per bead. For experiments, cell culture medium can be replaced with an appropriate buffer solution such as, e.g., HBSS.

In typical embodiments, the microsequential injection analysis of glucose uptake or lactate production is carried out using automated instrumentation. Methods for automated sequential injection analysis of cellular metabolism are known in the art. (See, e.g., Schultz and Ruzicka, supra; Schultz et al., supra). For example, studies can be carried out using a FIAlab automated sequential injection analyzer (e.g., FIAlab 3000; FIAlab Instruments, Medina, Wash.) with a multi-position (e.g., 6-position) lab-on-valve (LOV) manifold controlled by a bi-directional pump. In particular embodiments, a multi-position valve (MPV) with a dedicated syringe pump is added as a microbioreactor module. An assay is typically initiated by packing a column of cells attached to beads in the microbioreactor, which is upstream of the LOV flow cell. The cells-on-beads are perfused with the candidate compound in the buffer solution, followed by a stop-flow period (e.g., 120 s) to allow depletion of glucose and accumulation of lactate in the interstitial volume of the microbioreactor. Following the stop-flow period, a volume of the interstitial fluid from the cell column is injected through to the LOV cell (previously loaded with glucose or lactate reagent) and absorbance at 340 nm recorded.

In yet another embodiment of the present invention, biologically active agents are identified by evaluating the ability of the agents to inhibit oxygen consumption (e.g., inhibition of oxidative phosphorylation or inhibition of the electron transfer chain) in a cell line adapted to growth in a 2-methoxy antimycin derivative as described herein. Methods of measuring oxygen consumption in cells are generally known in the art. (See, e.g., Kumar et al., Arch. Biochem. Biophys. 420:169-175 (2003); Lou et cd., J. Exp. Biol. 203:1201-1210 (2000).) Generally, the method includes the following steps: (1) contacting a candidate compound to a cell adapted to growth in a 2-methoxy antimycin A derivative (a 2-methoxy antimycin resistant cell line); (2) contacting the candidate compound to a cell that is not adapted to growth in the 2-methoxy antimycin A derivative; (3) determining in each cell the level of oxygen consumption; and (4) determining whether the cell adapted to growth in the 2-MeOAA resistant cell line has a higher level of oxygen consumption relative to the cell that is not adapted to growth in the 2-MeOAA derivative.

In specific embodiments, the cell adapted to growth in the 2-MeOAA derivative (“2-MeOAA-adapted cell”) is adapted to growth in 2-methoxy antimycin A₁ or A₃. The 2-MeOAA-adapted cell is typically derived from a cell line such as, e.g., a RPMI-8226 cell line. Preferably, the cell that is not adapted to growth in the 2-MeOAA derivative (“non-adapted cell”) is of a cell line that is parental to the 2-MeOAA-adapted cell (i.e., the adapted cell is derived from the non-adapted cell line). Further, in certain embodiments, the 2-MeOAA-adapted and non-adapted cells are independently contacted with an inhibitor of oxidative phosphorylation or an inhibitor of the electron transfer chain such as, for example, an antimycin A (e.g., antimycin A₁ or A₃); and the level of oxygen consumption for each of the cells contacted with antimycin A is also determined. Inhibitors of oxidative phosphorylation or the electron transfer chain such as, e.g., antimycin A, inhibit oxygen consumption in both cells, while compounds having the desired activity (e.g., 2-MeOAA₁ or 2-MeOAA₃) only has this effect on the 2-MeOAA-adapted cell.

In other embodiments, combinatorial libraries of candidate compounds (e.g., antimycin derivatives) can be screened for biological activity using any of the methods described herein. For example, the methods can be used to identify combinatorial library compounds that modulate apoptosis by binding to the hydrophobic groove of a Bcl-2 family member protein. One such method for testing a candidate compound for the ability to bind to and potentially modulate apoptosis is as follows: (1) incubating at least one candidate compound from the combinatorial library independently with each of (a) an anti-apoptotic Bcl-2 family member protein and (b) a corresponding mutant Bcl-2 family member protein as described supra, such incubation for a time sufficient to allow binding of the combinatorial library compound to the protein; (2) removing non-bound compound from each of (a) and (b); (3) determining the presence of the candidate compound bound to (a) and (b); and (4) comparing the relative amounts of the candidate compound bound to (a) and (b) to determine whether the compound preferentially binds to the anti-apoptotic Bcl-2 family member protein relative to the corresponding mutant protein.

In a preferred embodiment, the agent (e.g., an antimycin derivative) exhibits reduced binding affinity for cytochrome b. Candidate compounds can be screened for such reduced binding affinity for cytochrome b. Methods for measuring binding to cytochrome B can, for example, include measuring the effect of the candidate compound on cytochrome bc₁ activity according to the methodology described by Miyoshi et al. (Biochim. Biophys. Acta 1229:149-154 (1995)). Briefly, submitochondrial particles are prepared from bovine heart mitochondria according to standard methods. (See, e.g., Matsuno-Yagi and Hatefi, J. Biol. Chem. 260:14424-14427 (1985).) The particles are treated with sodium deoxycholate (0.3 mg/mg protein) before dilution with reaction buffer. (See, e.g., Esposti and Lenaz, Biochim. Biophys. Acta 682:189-200 (1982).) Cytochrome bc₁ complex activity is measured at 30° C. as the rate of cytochrome c reduction with DBH as an electron donor. The reaction buffer can comprise 0.25 M sucrose, 1 mM MgCl₂, 2 mM KCN, 20 μM cytochrome c and 50 mM phosphate buffer (pH 7.4). The final mitochondrial protein concentration is 15 μg/ml.

In another embodiment, ATP production by mitochondria is measured as a measure of cytochrome b activity. For example, following an incubation (e.g., about 1 hour) of cells with the candidate compound, cells are harvested, and intracellular ATP concentrations are determined by, for example, an ATP-dependent luciferase-luciferin assay (Sigma, St. Louis, Mo.). An antimycin, such as A₁, and/or A₃, is used as a control. Reduced cytochrome b binding is indicated by a smaller reduction in intracellular ATP levels by the candidate compound than by the antimycin control.

The following examples are provided merely as illustrative of various aspects of the invention and shall not be construed to limit the invention in any way.

### EXAMPLES

### Example 1

To examine the sensitivity of cells over-expressing Bcl-x_{L} to various mitochondrial inhibitors and apoptosis inducers, cell lines over-expressing Bcl-x_{L} were prepared and tested.

Briefly, a DNA fragment encoding the full-length mouse Bcl-x_{L} cDNA was isolated from the plasmid pBS-BCL-x_{L} (Tzung et al., Am. J. Path. 150:1985-1995 (1997), incorporated herein by reference in its entirety) by digestion with the restriction endonuclease EcoRI. This EcoRI fragment was cloned into the EcoRI site of the mammalian expression vector pSFFV (Fuhlbrigge et al., Proc. Nat. Acad. Sci. USA 85:5649-5653 (1988)) in both sense and antisense orientations, to form expression plasmids pSFFV-Bc/-x_{L}-WT(sense) or pSFFV-Bc/-x_{L}(antisense), respectively. The tumorigenic murine hepatocyte cell line TAMH was transfected by lipofection (Lipofectamine, Life Technologies, Rockville, Md., according to the manufacturer's recommendations) with the plasmids pSFFV-neo (the control), pSFFV-Bc/-x_{L}-WT(sense) or pSFFV-Bc/-x_{L}(antisense). Characterization of and culture conditions for the cell lines have been previously published (Wu et al., Proc. Nat. Acad. Sci. USA 91:674-78 (1994); Wu et al., Cancer Res. 54:5964-5973 (1994)). Transfectants were selected for the acquisition of neomycin resistance by growing cells in the presence of 750 μg/ml of G418. Bulk transfectants were cloned by limiting dilution and individual clones were screened by immunoblot analysis to determine the level of Bcl-x_{L}, protein expression as described below.

Bcl-x_{L} protein expression was determined by Western blot analysis. Cell pellets or purified mitochondrial pellets were lysed in 1% Triton X-100, 5 mM Tris (pH 8.0) and 150 mM NaCl. Each lane was loaded with 20 μg of protein and electrophoresed (120 V) on a 12% SDS-polyacrylamide gel. Proteins were then electrically transferred to a nitrocellulose membrane. Immunodetection was performed using the rabbit anti-Bcl-x_{L} polyclonal antibody 13.6 (Gottschalk et al. Proc. Nat. Acad. Sci. USA 91:7350-7354 (1994)) followed by a biotinylated goat anti-rabbit antibody (Vector, Burlingame, Calif.; 1:500 dilution) and horseradish peroxidase conjugated streptavidin (Zymed, S. San Francisco, Calif.; 1:1000 dilution). Chemiluminescence (ECL, Amersham, Arlington Heights, Ill.) was used for detection. Expression of Bcl-x_{L} expression was indicated by the appearance of a band of approximately 29 kDa.

Bcl-x_{L} protein levels were determined by comparing the intensity of the 29 kDa band on a Western (immunoblot) blot between selected transfectants and the parental TAMH hepatocyte cell line. TABX2S cells (transfected with pSFFV.Bcl-x_{L} (sense)) was found to express a 4- to 5-fold higher level of Bcl-x_{L} protein as compared with the parental (control) TAMH.neo cells. The antisense transfectant TABX1A (transfected with pSFFV.Bcl-x_{L} (anti-sense)), on the other hand, was found to express little or no Bcl-x_{L} protein.

Mitochondrial expression of Bcl-x_{L} protein was examined by Western blot analysis of mitochondrial lysates prepared from TABX2S cells and TABX1A cells. Briefly, mitochondrial pellets were prepared by centrifugation and the pellets were lysed in 1% Triton, 5 mM Tris (pH 8.0) and 150 mM NaCl. Each lane of a 12% SDS-polyacrylamide gel was loaded with 20 μg of protein and electrophoresed (120 V) through the gel. Proteins were then electrically transferred to a nitrocellulose membrane. Detection of Bcl-x_{L} protein was as described above. Consistent with the results for overall cellular expression of Bcl-x_{L} protein, the level of mitochondrial Bcl-x_{L} protein was approximately 6 fold higher in TABX2S (pSFFV.Bcl-x_{L} (sense)) cells than TAMH.neo cells (control).

Selected transfectants were then tested for whole cell sensitivity to several apoptotic agents. Transfected cells were cultured to reach approximately 80% confluency prior to plating an equal number of cells from selected clones on 12-well tissue culture plates. The transplanted cells were treated with the following apoptotic agents: 5 μM doxorubicin for 48 hours; 5 μM cisplatin for 48 hours; or with 200 U/ml tumor necrosis factor (TNF) plus 1 μg/ml actinomycin D for 18 hours. Cell viability was determined by trypan blue dye exclusion. The percentage of viable cells was calculated by the number of viable cells (treated with a particular apoptogenic agent) divided by the number in the control group (untreated).

The sensitivity of the tested transfectants to treatment with apoptotic agents was inversely correlated with the level of Bcl-x_{L} expression. Cells over-expressed Bcl-x_{L} were less sensitive to the apoptogenic agent than control cells. For example, after treatment with doxorubicin (5 μM) for 48 hours, 50% of control TAMH.neo cells (control), 88% of TABX2S cells (over-expressing Bcl-x_{L}) and 20% of TABX1A cells (under-expressing Bch x_{L}) remained viable. A similar trend was observed with cisplatin or TNF treatment. Thus, cells which over-expressed Bcl-x_{L} were less sensitive to the apoptogenic agent than control cells, and conversely, cells which expressed an anti-sense construct, (pSFFV.Bcl-x_{L} (antisense)) were more sensitive than control cells.

TABX2S cells and TABX1A cells were also examined for the effects of various mitochondrial inhibitors. To test the apoptotic responses of these cells following direct perturbation of mitochondrial function, the cells were treated with rotenone (a mitochondrial complex I inhibitor), sodium azide (a mitochondrial complex IV inhibitor), antimycin A (a mitochondrial complex III inhibitor), valinomycin (an ionophore), and oligomycin (an ATP synthase or mitochondrial complex V inhibitor). Briefly, antimycin A (Sigma, St. Louis, Mo.) and rotenone were dissolved in dimethyl sulfoxide (DMSO) to form a stock solution, while valinomycin and oligomycin were dissolved in chloroform and ethanol, respectively, to form stock solutions. Azide was diluted from an aqueous stock solution. Antimycin A (a mixture of antimycins A₁-A₄) (0 to 5 μg/ml), rotenone (0 to 2.5 μg/ml), valinomycin (0 to 10 μg/ml), oligomycin (0 to 10 and azide (0 to 2 μM) were serially diluted into culture medium. Controls received an equivalent concentration of diluent. At various time points after drug treatment, cells were trypsinized, and cell viability was determined by trypan blue dye exclusion. The number of viable cells were counted and normalized to control group (i.e., % control=number of viable cells (treated group)/number of viable cells (control group)×100).

TABX2S cells were found to be markedly more sensitive than TABX1A and TAMH.neo cells to antimycin A over a wide range of concentrations. When the LD₅₀ of antimycin A was estimated from the dose-response curve, a seven-fold difference was found between TABX2S cells (LD₅₀=1.2 μM) and TABX1A or TAMH.neo cells (LD₅₀=8.3 μM). Following the addition of antimycin A to the cell culture, cell death was readily apparent within 2 hours in TABX2S cells, but not in TABX1A cells. The morphology of the dying cells was examined by light microscopy, which indicated that the TABX2S cells treated with antimycin A had an appearance consistent with apoptosis. The cells were also stained with Annexin V-EGFP and propidium iodide (PI), according to the manufacturer's instructions (Clontech, Palo Alto, Calif.). TABX2S cells treated with antimycin A exhibited a redistribution of phosphatidylserine to the outer plasma membrane, which is consistent with the induction of apoptosis. There were no significant differences in the sensitivity of the two cell lines to rotenone, sodium azide, valinomycin or oligomycin. Furthermore, the cell death induced by rotenone or valinomycin was not apparent until six to eight hours after treatment. Thus, cells over-expressing Bcl-x_{L} were more sensitive to antimycin A, but not to other mitochondrial inhibitors.

The effects of Bcl-x_{L} over-expression on non-tumorigenic cells was also examined. In particular, the sensitivity of cells that over-express Bcl-x_{L} to antimycin A was further examined in the non-tumorigenic mouse liver cell lines, AML-12 (ATCC CRL-2254) and NMH. Briefly, AML-12 cells were transfected as described above with pSFFV.Bcl-x_{L}(sense) and pSFFV.neo. AML-12-pSFFV. Bcl-x_{L}(sense) cells expressed approximately 3 to 4 fold higher Bcl-x_{L} protein levels than did AML-12 cells transfected with the control plasmid, pSFFV.neo, when assayed by Western blot analysis. The AML-12.Bcl-x_{L} cells demonstrated increased sensitivity to antimycin A, which is consistent with the results from TAMH cells. Similar results were also found with the mouse liver cell line NMH and with two other TAMH clones stably transfected with a vector that over-expresses Bcl-x_{L}. TAMH cells that over-express the related family member protein Bcl-2 were also more sensitive to antimycin A than were control cells.

Thus, cells which over-express Bcl-x_{L}, or Bcl-2 exhibited increased sensitivity to antimycin A. In particular, this inhibitor preferentially induced apoptosis in Bcl-x_{L}-over-expressing liver cell lines, confirming that certain mitochondrially active agents can overcome or bypass the anti-apoptotic effect of Bcl-x_{L} over-expression. Since over-expression of Bcl-x_{L} or Bcl-2 resulted in a decreased apoptotic sensitivity and has been implicated in multidrug resistance in cancer cells and carcinogenesis, this finding has clinical implications. In particular, this difference represents a significant therapeutic window which can be exploited for preferentially inducing apoptosis in cells over-expressing Bcl-x_{L} or Bcl-2, while cells which do not over-express Bcl-x_{L} or Bcl-2 are minimally affected.

### Example 2

In this example, various biochemical and biophysical indices associated with antimycin A treatment were examined and correlated with cell death. Specifically, reactive oxygen species (“ROS”) and ATP production were examined soon after initiating antimycin A treatment. Other parameters of mitochondrial function were also measured.

Electrons as reducing equivalents are fed into the mitochondrial electron transfer chain at the level of Coenzyme Q (CoQ) from the primary NAD⁺- and FAD-linked dehydrogenase reaction and are transferred sequentially through the cytochrome chain to molecular oxygen. As discussed above, antimycin A inhibits complex III (CoQH₂-cytochrome c reductase) downstream of CoQ. Complex III serves as an electron transfer station for transfer of electrons from CoQ to cytochrome c. Because CoQ is the major source of ROS derived from the mitochondrial respiratory chain (Turrens et al., Arch. Biochem. Biophys. 237:408-414 (1985)), inhibition of complex III often leads to increased ROS formation. The production of ROS in this example was measured by incubating control or antimycin A-treated cells with dihydroethidium. ROS present in the sample oxidizes dihydroethidium to the fluorescent product, ethidium (Rothe et al., J. Leukocyte Biol., 47:440-448 (1990)).

Briefly, TABX2S and TABX1A cells were harvested and resuspended at 5×10⁵ cells/ml. These cells were incubated with 5 μM dihydroethidium in tissue culture media for 45 minutes at 37° C. and then submitted for flow cytometric analysis. One hour after antimycin A treatment, when apoptosis was not apparent, the levels of ethidium were increased to a similar extent in both TABX2S and TABX1A cells. Similarly, when peroxide levels were measured by incubating the cells with dichlorodihydrofluorescein (H₂-DCF-DA), the increase in peroxide production was the same between the two cell lines. Thus, antimycin A treatment did not stimulate greater formation of ROS in antimycin A-sensitive (TABX2S) cells compared to antimycin A-resistant (TABX1A) cells.

Correlation of ATP production with cell death was examined by comparing the ATP levels in antimycin A-treated cells and control cells treated with DMSO vehicle alone. Similarly treated cells were tested for viability by trypan blue dye exclusion. Mitochondrial ATP production is driven by the electrochemical gradient generated along the respiratory chain. Following complex III inhibition by antimycin A, electron flow is blocked and ATP synthesis is interrupted.

To determine whether there was a negative correlation between ATP production and cell death, TABX2S and TABX1A cells were treated with (1) DMSO, (2) 2 μg/ml antimycin A, or (3) 2 μg/ml antimycin A plus fructose (50 mM added 15 minutes before and 15 minutes after administration of antimycin A). Fructose is a substrate that provides ATP production through the glycolytic pathway. After a 30 to 60 minute incubation, cells were harvested and intracellular ATP concentrations were determined by an ATP-dependent luciferase-luciferin assay (Sigma, St. Louis, Mo.). The ATP concentrations in DMSO-treated cells were taken as 100%. In parallel experiments, cell viability was determined after six hours.

Intracellular ATP levels were found to decrease by 70 to 75% in both TABX2S and TABX1A cells within 30 minutes of antimycin A treatment. Supplementation with fructose restored the ATP level to approximately 60% of control in both cell lines, but had no effect on subsequent cell death. Thus, ATP levels did not correlate with the extent of apoptosis. For instance, even though there was a higher ATP level in antimycin A-treated TABX2S cells supplemented with fructose than in antimycin A-treated TABX1A cells without fructose, significantly more apoptosis occurred in the former (33% survival vs. 87% survival). These data argue against a primary role of ATP depletion in mediating apoptosis in antimycin A-treated TAMH cells.

To further test if the mitochondrial respiratory chain in cells which over-express Bcl-x_{L} was more sensitive to antimycin A inhibition, cellular respiration was measured by oximetry. Briefly, TABX2S cells (over-expressing Bcl-x_{L}) and control cells (TAMH.neo) were suspended in air-equilibrated complete medium at a density of 3 million cells per milliliter and placed in a thermostatted electrode chamber at 37° C. The cells were treated with 1 μg/ml antimycin A. Polarographic measurements were made with a Clark-type oxygen electrode with continuous recording. Both cell types showed similar reductions in oxygen consumption. At higher concentrations of antimycin A, oxygen consumption was almost completely inhibited in TAMH.neo control cells, while TABX2S cells maintained about 20 percent of basal oxygen consumption. Thus, the sensitivity of cells which over-express Bcl-x_{L} to antimycin A was not a result of heightened effects on ATP levels, ROS generation or cell respiration.

The effect of antimycin A on mitochondrial function was further evaluated with the mitochondrial dye, JC-1 (5,5′,6,6′-tetrachloro-1,1′,3,3′-tetraethylbenzimidazolcarbo-cyanine iodide) (Molecular Probes, Eugene, Oreg.), a lipophilic, cationic carbocyanine dye, which has a fluorescence emission at 520 nm (green). JC-1 normally exists in solution as a monomer emitting a green fluorescence. When JC-1 assumes a dimeric configuration (J-aggregate) in a reaction driven by ΔΨₘ, it emits a red fluorescence (Reers et al., Biochem. 30:4480-86 (1991)). The use of JC-1 allows simultaneous analysis of mitochondrial volume (green fluorescence) and ΔΨₘ (red fluorescence). (See Mancini et al., J. Cell. Biol. 138:449-469 (1997)).

Briefly, at 15 and 30 minutes, 5×10⁵ cells were washed, trypsinized and resuspended in 1 ml of growth media. Each sample was stained with 10 μg/ml of JC-1 prepared in DMSO. After 10 minutes of incubation at 37° C., cells were transferred to ice and analysis was performed using a FACScan flow cytometer (Becton Dickinson). The excitation wavelength was 488 nm whereas measurement was performed at 520 and 585 nm for green and red fluorescence, respectively. Green and red fluorescence were measured on FL1 and FL2 channels, respectively. A minimum of 10,000 cells per sample were analyzed. Comparisons were made based on the results of at least three experiments.

There was a clear increase in JC-1 green fluorescence (mitochondrial volume), accompanied by a decline in JC-1 red fluorescence (mitochondrial transmembrane potential) in TABX2S cells one hour after antimycin A treatment. In contrast, JC-1 green and red fluorescence remained relatively unchanged in TABX1A cells. It should be noted that in the control cells (DMSO vehicle-treated cells), neither JC-1 green nor red fluorescence changed after one hour. When earlier time points were examined in TABX2S cells, there was already a significant increase (shift to right) in JC-1 green fluorescence as early as 15 minutes after addition of antimycin A, whereas JC-1 red fluorescence showed little change at this time. This finding suggested that the change of mitochondrial volume precedes that of ΔΨₘ.

The ultrastructural characteristics of TABX2S and TABX1A cells were further studied by electron microscopy. Briefly, cells were fixed in half strength Karnovsky's fixative and post-fixed in 1% collidine-buffered osmium tetroxide. After dehydration, cells were embedded in Epon 812. Ultrathin sections were stained using saturated aqueous uranyl acetate and lead tartrate and examined using a JEOL 100 SX transmission electron microscope operating at 80 kV. At two hours after exposure to antimycin A, TABX2S cells had become shrunken and displayed chromatin condensation and margination in the nuclei. These data confirm the apoptotic nature of the cell death. The mitochondria were markedly swollen with widening of the cristae, consistent with the increased JC-1 green fluorescence observed previously in this example. JC-1 staining, however, was found to be more sensitive in detecting mitochondrial changes because mitochondrial swelling was not apparent at 30 minutes or one hour when assayed by electron microscopy. The mitochondrial morphology was normal in the antimycin A-treated TABX1A (control) cells.

Mitochondrial PT is caused by opening of a large conductance channel in the inner mitochondrial membrane. Opening of a large conductance channel allows free distribution of solutes of less than 1,500 Da and results in dissipation of the proton gradient and osmotic swelling of mitochondria due to the higher solute concentration in the matrix. In isolated mitochondria, the colloid osmotic swelling associated with PT pore opening can be followed by measuring the optical density change at 540 nm (Kantrow et al., Biochem. Biophy. Res. Comm. 232:669-671 (1997)). Because antimycin A-treated TABX2S cells demonstrated increased JC-1 green fluorescence by flow cytometry and mitochondrial swelling by electron microscopy, which suggested the occurrence of PT, the effect of antimycin A on osmotic swelling of isolated mitochondria was tested.

Briefly, mitochondria were isolated from TABX2S cultured cells by a modification of the procedure of Maltese et al. (J. Biol. Chem. 260:11524-11529 (1985)). Typically, 0.5 to 1×10⁸ cells were harvested and washed once with homogenization buffer (250 mM sucrose, 10 mM Tris-HCl, 1 mM EDTA and 1 mg/ml BSA, (pH 7.4)). The cell suspension was exposed to nitrogen at 250 psi for 30 minutes in a “cell disruption bomb” (Parr, Moline, Ill.) or homogenized in a Dounce homogenizer with a loose-fitting pestle until >90% of cells were broken. The homogenate was centrifuged at 800×g for 10 minutes. The supernatant was removed and centrifuged at 10,000×g for 10 minutes at 4° C. The pellet was resuspended and again centrifuged at 10,000×g for 10 minutes. The mitochondrial pellet thus obtained was resuspended and adjusted to 0.5 mg protein/ml in an isotonic buffer consisting of 100 mM KCl, 75 mM mannitol, 25 mM sucrose, 5 mM Tris-phosphate, 10 mM Tris-HCl (pH 7.4), 0.05 mM EDTA and 5 mM succinate. For light scattering studies (e.g., for measurement of PT), the mitochondrial suspension was placed in a quartz cuvette, and continuous measurements of light absorption at 540 nm were obtained using a PerkinElmer Lambda 2 spectrophotometer.

Antimycin A added directly to the purified mitochondrial preparation at a concentration of 2 μg/ml caused mitochondrial swelling, which was detected by a rapidly occurring drop in absorbance at 540 nm in mitochondria prepared from TABX2S cells. A rapid fall in light absorbance is characteristic of large amplitude swelling. In contrast, mitochondria from TABX1A cells did not exhibit similar permeability changes and swelling, even at much higher concentrations of antimycin A. The addition of 100 mM CaCl₂ resulted in mitochondrial swelling of both TABX2S and TABX1A mitochondria. In contrast to these results with antimycin A, Bcl-x_{L}-expressing mitochondria were moderately resistant to calcium-triggered mitochondrial swelling.

Using the ΔΨₘ-sensitive JC-1 probe, the effects of antimycin A on isolated mitochondria were also tested. Isolated mitochondria were loaded with JC-1 prior to treatment, and mitochondrial labeling was determined using FACS. Relative to either initial mitochondrial red fluorescent staining or the basal fluorescence intensity of mitochondria treated with an uncoupler, CCCP, antimycin A caused a much greater decrease in ΔΨₘ of mitochondria having high levels of Bcl-x_{L} (TABX2S) than control mitochondria (TABX1A). Antimycin A-treated mitochondria with high levels of Bcl-x_{L} had lower levels of JC-1 staining than parallel samples treated with CCCP. Uncoupled mitochondria still retain a significant Donnan potential because of trapped anionic species and it is likely that antimycin-induced PT and/or swelling of mitochondria led to a further reduction of this residual potential. Mitochondria from TAMH.neo cells had an intermediate response to antimycin A.

In summary, examination of mitochondrial characteristics of transfected cells over-expressing Bcl-x_{L} in response to antimycin A demonstrated that ATP depletion and increased ROS production, which are direct consequences of complex III inhibition, did not correlate with cell death. Rather, antimycin A induced mitochondrial swelling in cells which over-express Bcl-x_{L}, as demonstrated by the flow cytometry and electron microscopy data discussed above. In addition, the findings that isolated mitochondria which over-express Bcl-x_{L} undergo rapid swelling after addition of antimycin A, while control mitochondria are completely resistant These data clearly demonstrated the local effect of Bcl-x_{L} in conferring antimycin sensitivity on mitochondria. Thus, antimycin A causes selective cell death by a mechanism independent of its mitochondrial complex III inhibition, but dependent on Bcl-x_{L} protein levels.

### Example 3

This example demonstrated that antimycin A-induced cell death was caspase independent. Bcl-2-like proteins can suppress apoptosis through direct and indirect effects on the cytosolic caspase-activating apoptosome complex (caspase-9, APAF-1 and cytochrome c) or by maintaining mitochondrial membrane integrity and osmotic homeostasis (Cosulich et al., Curr Biol. 9:147-150 (1999)). Thus, antimycin A could initiate apoptosis in Bcl-x_{L}-over-expressing cells by inducing Bcl-x_{L} to promote, rather than oppose caspase activation, possibly by altering interactions with APAF-1 (Pan et al., J. Biol. Chem. 273:5841-5845 (1998); Hu et al., Proc Nat. Acad. Sci. USA. 95:4386-4391 (1998)).

TABX2S and TABXIA cells were exposed to the broad spectrum caspase inhibitor, benzyloxycarbonyl-Val-Ala-Asp-fiuoromethyl ketone (zVAD-fmk). Antimycin A-induced death of TABX2S cells was found to be caspase-independent, as shown by the inability of zVAD-fmk to rescue such cells from cell death. This result indicated that the pro-apoptotic activity of antimycin A did not require caspase activity.

### Example 4

In this example the ability of antimycin A to promote mitochondrial depolarization in conjunction with Bcl-x_{L} expression was tested using a rhodamine 123 (“Rh-123”) retention assay (Petit et al., Eur. J. Biochem. 194:389-397 (1990); Imberti et al., J. Pharmacol. Exp. Ther. 265:392-400 (1993)). Rh-123 is a cationic lipid-soluble fluorescent dye that accumulates in mitochondria in proportion to the mitochondrial membrane potential. Mitochondria were isolated from TABX2S cells (over-expressing Bcl-x_{L}) and from control cells, prepared as described in Example 2. The isolated mitochondria were loaded with Rh-123 by incubation with 10 μM Rh-123 for 30 minutes, washing and resuspention in buffer. Five minutes after adding antimycin A, or a control diluent, the level of Rh-123 retained by the mitochondria was determined by flow cytometry. Less than 40% of Rh-123 was retained in antimycin A-treated TABX2S mitochondria, compared with greater than 80% retained in control mitochondria. These results indicated that antimycin A induced membrane depolarization, with rapid kinetics, in mitochondria from TABX2S cells, but not in control mitochondria.

### Example 5

To probe a potential interaction between antimycin A and Bcl-x_{L}, docking analysis was performed using the crystallographic structure of the Bcl-x_{L} protein and antimycin A coordinates from the NMR structure (Muchmore et al., Nature 381:335-341 (1996); Sattler et al., Science 275:983-986 (1997)) and the Available Chemicals Directory (Molecular Design, Ltd., San Leandro, Calif.). Further, a 3D structure has been determined by x-ray crystallography and NMR (Kim et al., J. Am. Chem. Soc. 121:4902-4903 (1999)). The program suite, DOCK (Kuntz, Science 257:1078-1082 (1992)), was used to determine if there was a compatible site on Bcl-x_{L} for binding of antimycin A and, if so, an optimal binding configuration. The DOCK program systematically moves the molecular structure of antimycin A along the surface of the Bcl-x_{L} structure and searches for a potential binding site based on shape complementarity, electrostatic interaction, hydrogen bond formation and other chemical energies. An optimal binding site was identified in the Bcl-x_{L} structure. Antimycin A was predicted to bind in an extended conformation to the hydrophobic pocket of Bcl-x_{L} formed by three conserved domains in the Bcl-2 family, BH1, BH2, and BH3. This binding site overlapped with the dimerization interface for Bak BH3 peptide and Bcl-x_{L} previously determined by NMR spectroscopy (Sattler et al., Science 275:983-986 (1997)). Two structures of the hydrophobic pocket have been used to probe the interaction of compounds. The first is the structure of the pocket when occupied by the BH3 peptide and the second is the structure of the pocket when not occupied. The depth of with of the pocket is reduced in the unbound state. This structure provides better contact calculations for potential compounds of interest, such as 2-methoxy antimycin A.

### Example 6

Based on the computer modeling prediction that antimycin A could directly bind to the hydrophobic pocket of Bcl-x_{L}, fluorescence spectroscopy was used to detect such a direct interaction. Antimycin A₃ exhibits a fluorescence maximum at 428 nm. The binding of antimycin A₃ to protein causes an increase in fluorescence intensity at the same wavelength providing a means for detecting the binding of antimycin A₃ to protein.

In this assay, 0 to 5 μM antimycin A₃ (Sigma Chemical Co., St. Louis, Mo.) was added to a physiological buffer (50 mM Tris-HCl pH 8.0, 0.2 M NaCl, 2 mM EDTA, 0.5% v/v glycerol) containing recombinant Bcl-2 or Bcl-x_{L} protein under conditions that permitted antimycin A₃ to bind to the BH3-binding domain of Bcl-2 or Bcl-x_{L}, (22.5° C. on a Hitachi F-2500 fluorescence spectrofluorimeter equipped with a thermostatted cell holder). Bovine serum albumin (BSA), which is known to bind antimycin A, and lysozyme, which does not, were used as positive and negative controls, respectively. The excitation wavelength was 335 nm, and the maximum emission wavelength for antimycin A₃ was 428 nm with a slit width of 10 nm. The samples were mixed in a quartz cuvette and checked for inner filter effect over the range of antimycin A₃ for this study. Blanks containing antimycin A₃ at the same concentration as the experimental samples were used as controls in all measurements and necessary background corrections were made.

Recombinant human Bcl-2ΔC22 (a recombinant human Bcl-x_{L} lacking the C-terminal 22-amino acid residue membrane anchor sequence) and mouse Bcl-x_{L}ΔC20 (a murine Bcl-x_{L} lacking the C terminal 20-amino acid residue membrane anchor sequence) fused with poly-His at the N-terminus were chromatographically purified to homogeneity. The concentrations of antimycin A₃ and stock solutions of recombinant proteins were quantitated using an extinction coefficient of 7.24/mM/cm at 320 nm and by Bradford assay, respectively. The stoichiometric ratio of antimycin A₃ and Bcl-2 producing the maximal change in antimycin A₃ fluorescence was determined with incremental addition of antimycin A₃ to a 1.98 μM solution of recombinant Bcl-2 in a volume of 2.1 milliliters. The change in volume resulting from the addition of antimycin A₃ was less than 5%. For peptide displacement experiments, a solution of 2 μM antimycin A₃ and 3 μM Bcl-2 was allowed to reach binding equilibrium at 4° C. prior to fluorescence measurements. Native peptide corresponding to the BH3 domain of Bak (72-GlyGlnValGlyArgGlnLeuAlallelleGlyAspAsp IleAsnArg-87 (SEQ ID NO:1)) or a mutant peptide with a single amino acid change (Leu78Ala-BH3) was added to the solution and the fluorescence measurements were repeated.

The fluorescence of the solution containing recombinant Bcl-2 and antimycin A₃ was increased above the fluorescence of antimycin A₃ alone, indicating that binding had occurred. The fluorescence intensity of antimycin A₃ also increased in the presence of BSA (the positive control), but not in the presence of lysozyme (negative control). The intrinsic fluorescence at 428 nm of antimycin A₃ increases by as much as 18% in the presence of Bcl-2 protein. The maximum change in fluorescence intensity of antimycin A₃ was observed at a molar stoichiometric ratio of antimycin A₃ to Bcl-2 of 1:1, as determined from a Job plot.

The BH3 peptide is also known to bind to the hydrophobic pocket of Bcl-x_{L} and Bcl-2. To determine if the site of antimycin A₃ interaction was the hydrophobic pocket of Bcl-2, a competitive binding assay was used. The relative concentrations of antimycin A₃ and Bcl-2 were adjusted to maximize formation of the antimycin A₃:Bcl-2 complex, as indicated by the fluorescence increase of antimycin A₃. BH3 peptide was then added to the preformed antimycin A₃:Bcl-2 complex, as described above. The fluorescence intensity of antimycin A₃ was inversely related to the concentration of BH3 peptide added. At a molar excess of BH3 peptide, antimycin A₃ fluorescence coincided with that of solutions of free antimycin A₃ (without Bcl-2), indicating the displacement of antimycin A₃ from Bcl-2. No overlapping fluorescence was observed from either the BH3 peptide or Bcl-2:BH3 peptide complex, and BH3 peptide alone did not affect antimycin A₃ fluorescence. BH3 peptide displaced antimycin A₃ from Bcl-2 polypeptide with an approximate Michaelis constant of 2.5 μM.

The ability of the mutant Bak BH3 peptide, Leu78Ala-BH3 (L78A-BH3), to displace antimycin A₃ bound to Bcl-2 polypeptide was also tested. The affinity of L78A-BH3 peptide for the Bcl-x_{L} hydrophobic pocket was diminished by two orders of magnitude compared to native Bak BH3 peptide. The L78A-BH3 peptide showed significantly reduced ability to displace antimycin A₃ from Bcl-2. Equivalent displacement of antimycin A₃ occurred at a forty fold higher concentration of L78A-BH3 peptide than that required for the native Bak BH3 peptide, which demonstrated the specificity of antimycin A₃-binding to the hydrophobic pocket of Bcl-2. The displacement of antimycin A₃ from Bcl-x_{L}, similarly required much higher concentrations of the L78A BH3 peptide. These results are consistent with the docking model in which antimycin A₃ is predicted to bind to Bcl-x_{L}, at the same binding site as the BH3 peptide the hydrophobic pocket.

### Example 7

The effects of antimycin A in TABX2S cells are similar to the reported mitochondrial and pro-apoptotic effects of peptides derived from the BH3 domain of Bax-like proteins (Chittenden et al., EMBO J. 14:5589-5596 (1995); Cosulich et al., Curr Biol. 7:913-920 (1997); Holinger et al., J Biol. Chem. 274:13298-13304 (1999)). Based on this observation, the Bak-derived BH3 peptide was tested to determine if it also selectively depolarized mitochondria from TABX2S cells (over-expressing Bcl-x_{L}).

In this experiment, the synthetic 16-residue Bak BH3 peptide (SEQ ID NO: 1; Example 6) was added to mitochondria from TABX2S cells (over-expressing Bcl-x_{L}) and to control cells. The addition of the Bak BH3 peptide at 3.5 μM induced similar Rh123 dye leakage by TABX2S mitochondria as that produced by antimycin A. Mitochondria from TABX1A cells were minimally affected by the same concentration of BH3 peptide, or by antimycin A. Thus, antimycin A acts like Bak BH3 peptide in inducing membrane depolarization. Although high levels of Bcl-x_{L} maintain mitochondrial integrity in intact cells or isolated organelles exposed to a wide range of stressors, the addition of antimycin A or Bak BH3 peptide overcomes this resistance to depolarization. In contrast, the control cells, which express Bcl-x_{L} at physiological levels, were resistant to BH3 peptide-induced membrane depolarization.

This dichotomy can perhaps best be explained by the specific interaction of pro-apoptotic BH3 peptides with the hydrophobic groove in the Bcl-x_{L} structure (Sattler et al., Science 275:983-986 (1997)). Reduced levels of Bcl-x_{L}, resulted in a lower number of binding sites for BH3 peptides and resistance to BH3-mediated effects. A similar mechanism may explain the specific effects of antimycin A on Bcl-x_{L}-expressing mitochondria. These results suggest that antimycin A acts as a molecular mimic of endogenous pro-apoptotic proteins. Low expression of Bcl-x_{L} reduced the mitochondrial toxicity of both antimycin A and BH3 peptide.

### Example 8

In this example the ability of antimycin A to prevent pore formation by Bcl-x_{L}, was tested. The Bcl-x_{L} protein has reversible pore-forming activity. Recombinant human Bcl-x_{L} lacking the C-terminal 20-residue membrane anchor sequence, Bcl-x_{L}ΔC20, forms pores in large unilamellar vesicles. A reporter, calcein, can leak out of the vesicles through these pores. If antimycin A affects Bcl-x_{L}ΔC20 pore formation, the leakage of calcein will change, as can be measured by a change in fluorescence.

Large unilamellar vesicles composed of 60% dioleoylphosphatidylcholine and 40% oleoylphosphatidylglycerol were prepared by the extrusion method of Mayer et al. (Biochim. Biophys. Acta 858:161-168 (1986)). Briefly, a dry film of lipid was resuspended in an aqueous solution containing 40 mM calcein (Molecular Probes, Eugene, Oreg.), 25 mM KCl and 10 mM HEPES (pH 7.0). After 5 freeze-thaw cycles, the lipidic solution was extruded through 2 Nucleopore filters, 0.1 μm pore diameter. Nonencapsulated material was removed from the vesicles using a SEPHADEX G-50 column (Pharmacia, Uppsala, Sweden), with 10 mM HEPES (pH 7.0), 100 mM NaCl, as the elution buffer. The size of the vesicle suspension was measured by a Coulter N4 Plus-Sizer to confirm that the mean diameter of the vesicle sample was close to the expected size (100 nm). The osmolalities of all solutions were measured in a cryoscopic osmometer (Wescor Inc., Logan, Utah) and adjusted to 0.21 Osmol/kg by the addition of sodium chloride, as necessary. Lipid concentration was measured as described previously (Stewart, Anal. Biochem. 104:10-14 (1989)).

Calcein leakage was determined by adding 2-4 μg of purified Bcl-x_{L}ΔC20 (5 μg/ml, 161 nM) to a solution of 100 mM NaCl, 10 mM HEPES (pH 5.0) containing the large unilamellar vesicles (50 μM final lipid concentration) described above. Changes in the fluorescence intensity were measured in an Aminco-SLM spectrofluorimeter. BH3 peptides and antimycin derivatives were incubated with Bcl-x_{L} for 5 minutes prior to addition to the liposome suspension. Assays were performed at 37° C. in a thermostatted cuvette with constant stirring. Excitation and emission wavelengths for calcein were 495 nm and 520 nm, respectively, at a slit width of 4 nm. The 100% fluorescence level for leakage was obtained by detergent lysis (0.1% Triton X-100) of the vesicles containing entrapped calcein.

In vesicles preloaded with calcein, about 40% of the reporter leaks from the vesicles within about 3 minutes of Bcl-x_{L}ΔC20 addition. Leakage of calcein was inhibited in a dose-dependent fashion by antimycin A. At a concentration of 12 μM, antimycin A completely blocked Bcl-x_{L} pore-forming activity.

The ability of the Bak BH3 peptide to inhibit leakage of calcein was also tested. Native BH3 peptide inhibited Bcl-x_{L}-induced calcein efflux from synthetic liposomes, with 50% inhibition at about a 20:1 molar ratio of Bak BH3 peptide:Bcl-x_{L} protein. This inhibition is equivalent to the approximately 20:1 molar ratio of antimycinA:Bcl-x_{L} that is required to achieve a 50% inhibition of calcein leakage. In contrast, the mutant L78A-8H3 peptide has a minimal effect on Bcl-x_{L}-induced pore formation even at a 100-fold molar excess. Thus, antimycin A is capable of blocking the ability of Bcl-x_{L} to act as a membrane pore.

### Example 9

Studies of cellular respiration, ATP levels and reactive oxygen species in antimycin A-treated cell lines strongly suggested that the observed differences in cell viability could not be explained by the known effects of antimycin A on mitochondrial electron transfer or oxidative phosphorylation. To definitively address whether the Complex III inhibitory activity of antimycin A was involved in the selective death of cells over-expressing Bcl-x_{L}, a structure-activity relationship for antimycin A₃ was determined.

In this example, two derivatives of antimycin A₃ were prepared, antimycin A₃ methyl ether (2-methoxy ether antimycin A₃) and phenacyl ether antimycin A₃. The structure of antimycin A₃ was shown above (Formula (I), where R₁ is a butyl group). (See also van Tamelen et al, J. Am. Chem. Soc. 83:1639 (1961)). Antimycin A₃ methyl ether has the following Formula (VIII) and an absolute configuration of [2R, 3R, 4S, 7S, 8R]:

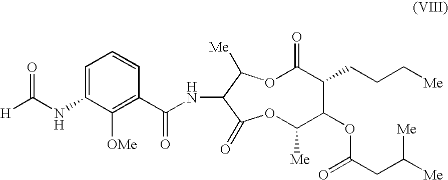

Antimycin A₃ methyl ether is prepared directly from antimycin A₃ as follows: Briefly, antimycin A₃ (14.0 mg) was dissolved in ethyl ether and a stream of diazomethane was passed through the reaction mixture until the yellow color persisted. The reaction mixture was treated with acetic acid until it became colorless. The mixture was reduced to dryness under reduced pressure and chromatographed on a silica gel to yield 14.3 mg of antimycin A₃ methyl ether. The resulting product was characterized by NMR, infrared spectroscopy and mass spectroscopy.

The phenacyl ether derivative of antimycin A₃ was prepared as follows: A solution of antimycin A₃ (5.7 mg, 10.95 mmol) in dry acetonitrile was treated with phenacyl bromide (4.4 mg, 21.9 mmol) and powdered potassium carbonate (6.0 mg, 43.8 mmol). The mixture was allowed to stir at room temperature for 18 hours. The reaction mixture was applied directly to a silica gel chromatography column. The product was eluted with 20% ethyl acetate/hexane to yield 5.4 mg (78%) of the product as a colorless oil. The resulting product was characterized by NMR, infrared spectroscopy and mass spectroscopy.

### Example 10

The antimycin A₃ methyl ether derivative prepared in Example 9 was studied to determine its affect on the apoptotic pathway in cells over-expressing Bcl-x_{L}. The methyl ether derivative was previously shown to be inactive as an inhibitor of cytochrome bc₁. (See, e.g., Miyoshi et al., Biochim Biophys Acta 1229:149-154 (1995); Takotake et al., Biochim Biophys Acta 1185:271-278 (1994).) The methyl ether also has a negligible effect on cellular O₂ consumption compared to the original antimycin A₃ compound. TABX2S (over-expressing Bcl-x_{L}), TAMH.neo (control) and TABX1A (antisense) cell lines were treated with 2-methoxy antimycin A₃. This derivative exhibited selective cytotoxicity for cells that over-express Bcl-x_{L}, but not for control cells. This pattern was identical to that seen with antimycin A₃, indicating that inhibition of cellular respiration by this antimycin was not required for Bcl-x_{L}-related apoptosis.

To confirm these data, assays were also performed with mitochondrial fractions from each cell line using the mitochondrial probe JC-1. Mitochondria from cells over-expressing Bcl-x_{L} (TABX2S cells) were strongly depolarized after addition of the 2-methoxy derivative at a concentration of 2 μg/ml. As observed for the parent compound, antimycin A₃, mitochondria with normal levels of Bcl-x_{L} expression were not affected by the 2-methoxy analog.

Finally, the 2-methoxy antimycin A₃ derivative was shown to bind recombinant Bcl-2 protein. 2-methoxy antimycin A₃ derivative is non-fluorescent due to the additional electrophilic substituent on the benzene ring. Thus, binding of 2-methoxy antimycin A₃ to the Bcl-2 protein can be measured in a competitive binding assay by monitoring fluorescence from antimycin A₃. For these experiments, antimycin A₃ (2 μM) and either 2-methoxy ether antimycin A₃ or phenacyl ether antimycin A₃ (2 μM) were added simultaneously to Bcl-2 polypeptide (3 μM) and allowed to equilibrate for 7.5 minutes at 22.5° C. before measuring the fluorescence intensity of antimycin A₃. The fluorescence of a prebound antimycin A₃-recombinant Bcl-2 complex decreased exponentially with the addition of 2-methoxy antimycin A₃, indicating competition for the antimycin A₃ binding site on Bcl-2. As an additional control for binding specificity, the effect of the phenacyl ether derivative of antimycin A₃ was also tested. Although of similar hydrophobicity, the phenacyl ether derivative did not displace antimycin A₃ from Bcl-2. These results strongly suggest that the cellular and mitochondrial sensitivity to antimycin A₃ in Bcl-x_{L}-expressing cell lines resulted from direct binding of antimycin A₃ to Bcl-x_{L} protein. Furthermore, the 2-methoxy ether antimycin A₃ derivative inhibited Bcl-x_{L} pore formation in a liposome permeability assay almost as well as antimycin A₃.

The results demonstrate that the antimycins have two structurally distinguishable protein-binding activities, one for binding to cytochrome bc₁, and the other for binding to Bcl-2 family member proteins, and that these activities are separable.

### Example 11

### Characterization of Bcl-x_{L} Mutant Proteins

Bclx_{L} mutants were derived from pSFFV-Bcl-x_{L}-WT (Example 1) using site directed mutagenesis (QuikChange XL, Stratagene). Briefly, mutagenic primers spanning each target site were used to amplify fragments containing the desired mutations. Residual wild-type template was then removed by digesting with the methylation-dependent endonuclease, DpnI. For recombinant expression, Bcl-x_{L}CΔ22 lacking the COOH-terminal membrane anchor sequence was generated by PCR. PCR products were digested with NdeI and XhoI and ligated into pET22b(+) (Novagen). All constructs were confirmed by sequence analysis.

### Staurosporine (STS) and Antimycin Toxicity in TAMH Cells Over-Expressing Bcl-x_{L} Mutants.

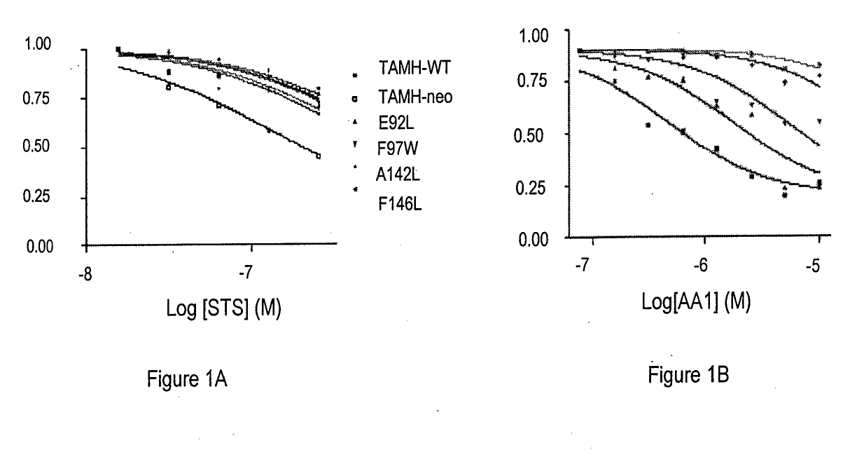

TAMH cells were transfected with DNA encoding each of the mutant Bcl-x_{L} proteins by lipofection (See Example 1 for method). For analysis of Bcl-x_{L} expression, 20 μg of cell protein was separated by 20% SDS-PAGE and transferred to nitrocellulose membranes. Immunodetection of Bcl-x_{L} was carried out using rabbit anti-Bcl-x_{L} antibody and Protein A/horseradish peroxidase conjugate, followed by chemiluminescent detection. Cells were grown to about 80% confluence in 96-well plates followed by addition of 100 μl of 2×AA₁ or staurosporine (STS) solution in complete medium. STS is a natural product originally isolated from the bacterium Streptomyces and found to be capable of inducing apoptosis in certain cells and which induction of apoptosis could be blocked by the expression of a Bcl-2 family protein. Cell viability was determined spectrophotometrically after 24 h treatment as the ratio of reduced and oxidized Alamar Blue (BIOSOURCE) at 570 nm and 600 nm, respectively. All results were normalized against DMSO controls. LD₅₀ values were calculated by non-linear regression analysis using Prism software (Graphpad). Results are shown in FIG. 1. Similar results were obtained using sulforhodamine B assays for total cell protein.

### Recombinant Bcl-x_{L} Purification.

A pET22b (Novagen) vector coding for Bcl-x_{L}(ΔC), a mutant Bcl-x_{L} protein without the COOH terminal region, was transformed into Escherichia coli BL21(DE3) cells that carried pUB520 (encoding human Arg tRNA) and grown to an A₆₀₀ of 0.6. Protein expression was induced with 0.1 mM isopropyl β-D-thiogalactoside at 30° C. The cells were resuspended 1:5 (w/v) in PEB buffer (50 mM Tris, pH 8.0, 200 mM NaCl, 0.2 mM phenylmethylsulfonyl fluoride, 5 mM β-mercaptoethanol, 5 mM imidazole, and 1% (v/v) glycerol), and stirred for 20 min at 4° C. Cells were disrupted by pulse sonication, and the soluble fraction was loaded onto a nickel-nitrilotriacetic acid column (Qiagen) equilibrated with PEB buffer. The column was washed with 40 mM imidazole, eluted with 200 mM imidazole, and the protein fractions were pooled and dialyzed (50 mM Tris, pH 8.0, 200 mM NaCl, 0.2 mM phenylmethylsulfonyl fluoride) at 4° C. overnight. The dialyzed protein was concentrated to 10 mg/ml and fractionated on a Superdex 75 gel filtration column (Amersham Biosciences). The fractions containing Bcl-x_{L}(ΔC) protein were pooled, exchanged into low-salt buffer (same as for previous dialysis buffer, except 50 mM NaCl), and loaded onto a MonoQ anion exchange column (Amersham Biosciences). Protein was eluted from the column with increasing NaCl gradient, pooled and concentrated. Purity was >99% as determined by silver staining, Bcl-x_{L}(ΔC) protein concentrations in 6 M guanidine HCl were determined from 280 nm absorbance using extinction coefficients of 41940 M⁻¹ cm⁻¹ for WT, E92L, A142L, and F146L, and 47630 m⁻¹ cm⁻¹ for F97W Bcl-x_{L}(ΔC) proteins.

### Binding of Antimycin A1 to Wild-Type and Mutant Bcl-x₁, Proteins.

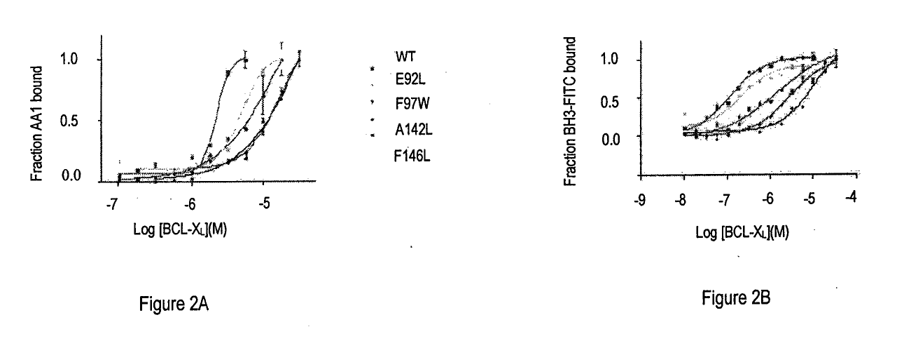

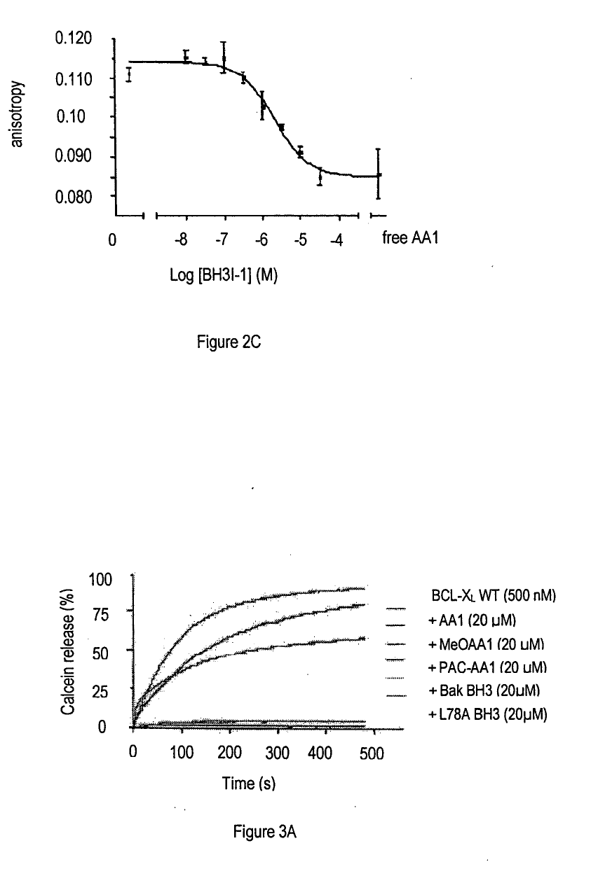

Fluorescence anisotropies of AA₁ and FITC-labeled BAK BH3 peptide were measured using a Fluoromax-3 spectrometer equipped with autopolarizers. All reagents were prepared in 0.2 μm-filtered PBS with fresh 1 mM DTT. Excitation and emission wavelengths were 340 nm and 420 nm for AA₁, and 480 nm and 520 nm for FITC-labeled BAK BH3 peptide respectively. Slit widths were set at 10 nm for both excitation and emission. AA₁ (200 nM) or BH3 peptide (25 nM) were equilibrated with different concentrations of Bcl-x_{L}(CΔ22) and mutant Bcl-x_{L} proteins for at least 1 h at room temperature. Each data point represents the mean of three independent measurements. Fluorescence anisotropy values were converted to fraction of ligand bound (f_{B}) and expressed on a semi-log plot with non-linear curve fitting. (Lakowicz, Principles of Fluorescence Spectroscopy, 2nd Ed., pp 308-309, Kluwer Academic/Plenum Publishers, New York). Results are shown in FIG. 2.

### Antimycin Inhibition of Pore-Forming Activity of Wild-Type and Mutant Bcl-x₁, Proteins.

Large unilamellar vesicles composed of 60% dioleoylphophatidylcholine and 40% dioleoylphosphatidylglycerol were prepared by the extrusion method of Mayer et al., supra. Lipid stocks, in chloroform, were mixed and dried under a stream of nitrogen gas. The lipid was resuspended by vortexing for 30 min in a solution of 40 mM calcein (Molecular Probes), 25 mM KCl, and 10 mM KOAc, pH 5.0. After 10 freeze-thaw cycles, the lipid suspension was extruded through two 0.1 μm pore diameter Nucleopore filters. Non-entrapped dye was removed by passage over a Sephadex G10 column (Amersham Biosciences). Lipid concentration was measured using the ammonium ferrothiocyanate method (Stewart, Anal. Biochem. 104:10-14, 1980).

For pore assays, recombinant truncated Bcl-2 family protein or mutant protein (Bcl-x_{L}(ΔC)) (500 nM) was added to large unilamellar vesicles (60 μM lipid concentration) in 100 mM KCl, 10 mM KOAc, pH 5.0, and fluorescence measured (490 nm excitation, 520 nm emission) with a Fluoromax-3 spectrophotometer. Peptides of the BH3 domain were incubated with the Bcl-x_{L}(ΔC) 5 min prior to mixing with liposomes. AA₁ was added to liposomes 1 min before adding the Bcl-x_{L}(ΔC) protein. Samples for kinetic assays were analyzed in a thermostatted cuvette at 37° C. with constant stirring. Dose responses were measured in black quartz microplates (Hellma) at room temperature. Calcein release was expressed as percentage of maximum release with detergent lysis (0.1% Triton X-100). Pore inhibition was calculated using cumulative dye release normalized to results obtained in absence of inhibitors, and the IC₅₀ values were determined by non-linear regression analysis. Results are shown in Table 4.

### Crystallographic Studies.

Purified wild-type and mutant Bcl-x_{L}(ΔC) proteins were concentrated to 1 mM and crystallized by hanging drop vapor diffusion at 4° C. The mother liquor consisted of 50 mM MES, pH 6.0., 1.9 M ammonium sulfate. Crystals were flash-frozen in liquid nitrogen after soaking in mother liquor plus 30% trehalose (Sigma) for 1 min. Data sets were collected at 100 K with a Rigaku x-ray generator (100 mA and 50 kV) and a Raxis IV imageplate. DENZO and SCALE-PACK (Otwinowski and Minor, in Macromolecular Crystallography (Charles, et al. eds.), pp. 307-326, Academic Press, San Diego, Calif., (1997)) were used to process the diffraction data.

The program EPMR (Kissinger et al., Biol. Crystall. D 55:484-491 (1999)) was used to find a molecular replacement solution using the Bcl-x_{L}^{wt} structure (Protein Data Bank code 1MAZ) as a starting model. The space group for Bcl-x_{L} and all mutant proteins was determined to be P4₁,2₁,2₁. A free R set (Bruger, Nature 355:472-475 (1992)) of 10% was set aside using the CCP4 program FreeRflag. The Xfit 4.0 program from the Xtalview suite (McRee, J. Struct. Biol. 125:156-165 (1999)) was used to visualize and modify the structure. The CNS (Brunger et al., Biol. Crystall. D 54:905-921 (1998)) program package was used for model refinement and simulated annealing composite omit 2FₒF_{c} maps were used to guide model rebuilding. The stereochemical properties of all structures were examined by PROCHECK (Laskowski et al., J. Appl. Cryst. 26:283-291 (1997)). Subsequent structural alignments, analysis, and figures were done with Swiss PDB viewer (Guex and Peitsch, Electrophoresis 18:2714-2723 (1997)), with pictures rendered using POVRay (available at the website for Persistence of Vision Raytracer, Pty. Ltd.). A summary of crystallographic statistics is provided in Table 4.

TABLE 4
Summary of Crystallographic Statistics.

| | | WT | E92L | F97W | A142L | F146L |
|---|---|---|---|---|---|---|
| Data Statistics | | | | | | |
| Unit Cell | (a) | 63.34 | 63.39 | 64.39 | 63.3 | 63.19 |
| | (b) | 63.34 | 63.89 | 64.39 | 63.3 | 63.19 |
| | (c) | 109.82 | 109.29 | 110.98 | 110.14 | 109.87 |
| Space Group | | P4₁2₁2 | P4₁2₁2 | P4₁2₁2 | P4₁2₁2 | P4₁2₁2 |
| Resolution (Å) | | 1.95 | 2.1 | 2.7 | 2.2 | 2.0 |
| Completeness (%) | | 94.3 | 99.9 | 95.9 | 98.4 | 98.9 |
| R_{merge}(%) | | 3.3 | 3.6 | 7.7 | 7.0 | 4.0 |
| Refined Statistics | | | | | | |
| R_{cryst} | | 20.3 | 21.2 | 19.1 | 20.5 | 20.8 |
| R_{free} | | 21.6 | 23.9 | 23.0 | 23.4 | 20.8 |
| Test Size (%) | | 10 | 10 | 10 | 10 | 10 |
| No. Mol. in Asym Unit | | 1 | 1 | 1 | 1 | 1 |
| No. of non-hydrogen atoms | | | | | | |
| Protein | | 1154 | 1154 | 1156 | 1158 | 1155 |
| Water | | 235 | 143 | 16 | 100 | 222 |
| RMSD from ideal values | | | | | | |
| Bond lengths (Å) | | 0.0075 | 0.0070 | 0.0070 | 0.0054 | 0.0072 |
| Bond angles (°) | | 1.067 | 1.186 | 1.179 | 1.104 | 1.203 |
| Ramachandran plot (%) | | | | | | |
| Most favored regions | | 96.9 | 94.5 | 89.0 | 95.3 | 92.1 |
| Additional allowed regions | | 3.1 | 4.7 | 10.2 | 3.9 | 7.9 |
| Generously allowed regions | | 0 | 0.8 | 0.8 | 0.8 | 0 |
| Disallowed regions | | 0 | 0 | 0 | 0 | 0 |

A series of point mutations were introduced to alter specific residues in the BCL-x_{L}, hydrophobic groove contact with AA from the docking model. The following single amino acid substitutions were made in human Bcl-x_{L}: E92L, F97W, A142L, and F146L. Stable transfectants of TAMH murine hepatocytes for each of the mutated Bcl-x_{L} plasmids as well as wild-type Bcl-x_{L}. Mutant Bcl-x_{L} (Bcl-x_{L}^{wt}) and wild type proteins (Bcl-x_{L}^{wt}) were expressed at similar levels.

To assess the effect of mutations of Bcl-x_{L} function, TAMH/Bcl-x_{L}^{mu} cells were tested for survival during STS treatment. Dose-response curves show that each of the Bcl-x_{L} mutant proteins produced equivalent levels of protection against STS-induced cell death. LD₅₀ values for STS with cells expressing Bcl-x_{L} mutants were not significantly different from Bcl-x_{L} wild-type cells (LD₅₀=0.58±0.1 μM). Vector-only control cells expressed low levels of endogenous Bcl-x_{L} and were significantly more sensitive to STS (LD₅₀ 0.11±0.01 μM) than any of the Bcl-x_{L}, or Bcl-x_{L} mutants cell lines (p<0.05). (Table 5).

The Bcl-x_{L} and Bcl-x_{L} mutant cells were next challenged with antimycin A₁. In contrast to the results with STS, antimycin A sensitivity varied substantially among the Bcl-x_{L} mutant cell lines. Compared with Bcl-x_{L} wild-type cells (LD₅₀=0.47±μM) the E92L and F97W Bcl-x_{L} mutants had reduced sensitivity to antimycin (LD₅₀=1.72±0.3 μM and 5.12±0.9 μM, respectively), whereas the A142L and F146L Bcl-x_{L} mutant cells were completely insensitive to antimycin A₁. (Table 5).

AA-Insensitive Bcl-x_{L} Mutants have Lower Binding Affinity for Antimycin A₁.

Recombinant Bcl-x_{L}, mutant mutants (Bcl-x_{L}^{mu}(ΔC)) and wild-type proteins were purified from bacterial extracts by nickel-nitrilitriacetic acid affinity gel filtration, and anion-exchange column chromatography. Direct quantitative measurements of AA₁ binding to Bcl-x_{L}(ΔC) proteins were obtained from fluorescent anisotropy under equilibrium conditions. Binding constants were calculated using non-linear regression analysis. (See Table 4). AA₁ has a much weaker binding affinity with the F97W, A142L and F146L mutants (K_{d}=17.56±5.2 μM, 41.77±21.4 μM, and 20.04±9.4 μM. respectively) than with Bcl-xMAC) protein (2.36±1.41 μM). The binding affinity of AA₁ with the E92L mutant (K_{d}=5.06±0.86 μM) was reduced 2- to 3-fold relative to Bcl-x_{L}^{wt}. Notably, the ranking of in vitro AA₁ binding affinities for the Bcl-x_{L}^{mu}(ΔC) proteins is in register with the in vivo sensitivities to AA.

TABLE 5
Summary of AA₁ activity on wild-type and mutant Bcl-x_{L} proteins.

| Cytotoxicity | | | Dissociation Constant | | Pore Inhibition | |
|---|---|---|---|---|---|---|
| | STS LD₅₀ | AA₁ LD₅₀ | | BH3 K_{D} | AA₁ IC₅₀ | BAK BH3 |
| Bcl-x_{L} | (μM) | (μM) | AA₁ K_{D} (μM) | (μM) | (μM) | IC₅₀ (μM) |
| WT | 0.58 ± 0.14 | 0.47 ± 0.07 | 2.36 ± 1.41 | 0.11 ± 0.029 | 2.03 ± 0.24 | 0.58 ± 0.36 |
| E92L | 0.52 ± 0.12 | 1.72 ± 0.34² | 5.06 ± 0.86¹ | 0.22 ± 0.05 | 2.47 ± 0.30 | 2.00 ± 0.87 |
| F97W | 0.34 ± 0.10 | 5.12 ± 0.94² | 17.56 ± 5.20² | 2.89 ± 0.41² | 3.45 ± 0.34² | 71.61 ± 9.01² |
| A142L | 0.49 ± 0.11 | >10 | 41.77 ± 21.49¹ | 13.00 ± 5.57¹ | 4.10 ± 1.24¹ | ND |
| F146L | 0.38 ± 0.04 | >10 | 20.04 ± 9.41¹ | 1.26 ± 3.57² | 3.60 ± 0.40² | 10.62 ± 3.15² |

¹indicates p < 0.05
²indicates p < 0.01
ND indicates that the non-linear regression was unable to fit this curve due to a lack of inhibition

The non-peptide Bcl-x_{L} inhibitors, BH3I-1 and BH3I-2, interact with Phe-97 and Ala-142 in the Bcl-x_{L} hydrophobic pocket by NMR chemical shift perturbation (Degterev et al., Nat. Cell Biol. 3:173-182 (2001); Lugovskoy et al., J. Amer. Chem. Soc. 124:1234-1240 (2002)). It was determined that BH3I-1 competes with AA₁ for Bcl-x_{L}(ΔC) binding. The K, for Bh3-I-1 displacement of AA bound to Bcl-x_{L}(ΔC) was 1.874±0.617 μM, similar to that reported for displacing BH3 peptide (Degterev et al., supra).

The affects of the hydrophobic groove mutations on binding of BAK BH3 domain peptides was also determined. Fluorescent anisotropy measurements were conducted using the FITC-labelled 16-residue BAK-BH3-peptide (SEQ ID NO: 1). The F97W, A142L, and F146L mutations resulted in substantially diminished BH3 peptide binding compared with Bcl-x_{L}^{wt}(ΔC). Interestingly, the relative affinities of BAK BH3 peptide with the Bcl-x_{L}^{mu}(ΔC) proteins (Bcl-x_{L}^{wt}>E92L>F97˜W F146L>A142L) paralleled those determined for AA.

### Pore Forming Activities of Mutant Bcl-x_{L} Proteins.

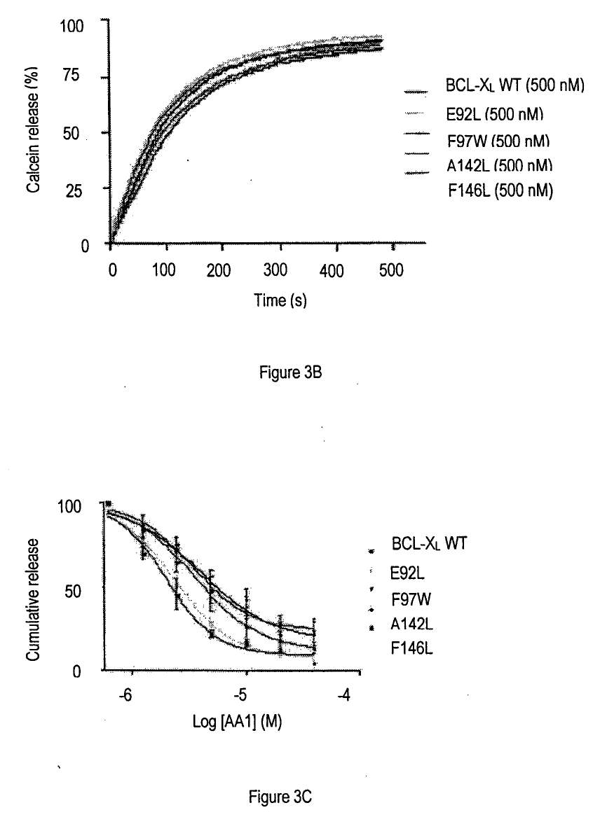

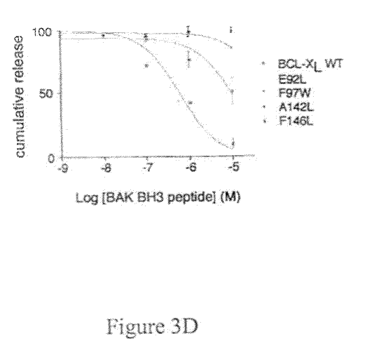

Synthetic lipid vesicles were loaded with the self-quenching fluorescent dye, calcein, to measure membrane pore formation b recombinant Bcl-x_{L}(ΔC) proteins as previously described. Addition of AA₁, 2-OMeAA₁, or the BAK BH3 peptide inhibited Bcl-x_{L} pore-forming activity, whereas a modified antimycin bearing a bulky phenacyl substituent, a mutated BAK peptide (L78A) with low Bcl-x_{L} affinity and BH3I-1 had no effect. (FIG. 3A).

The mutant versions of Bcl-x_{L}(ΔC) had similar pore-forming properties as Bcl-xMAC) (FIG. 3B). The sensitivity of Bcl-x_{L}^{mu}(ΔC) pores to AA₁ and to BAK BH3 peptide was measured under similar experimental conditions (FIGS. 3C and 3D) and non-linear regression analysis of the dose-response curves was performed to obtain IC₅₀ values (Table 4). Although the clustering of AA₁ IC₅₀ values in the liposome assay was tighter than observed for the binding affinities of AA₁ using soluble Bcl-x_{L}(ΔC), the same ordering of responses was obtained: WT>E92L>F97W˜F146L A142L. The BAK BH3 peptide ICₛₒ values also exhibited the same ordering observed for the binding affinities obtained from the equilibrium binding assay.

Preservation of Tertiary Fold with Mutant Bcl-x_{L} Proteins.

The structures of Bcl-x_{L}^{wt}(ΔC) and the four Bcl-x_{L}^{mu}(ΔC) proteins, E92L, F97W, A142L, and F146L, were solved by x-ray crystallography. Overall, the mutations produced only local effects on the wild-type structure. An α-carbon overlay of the wild-type protein with the mutant structures was carried out. The largest differences were primarily localized to the α3 helix between residues Tyr-101 and His-113. The RMSD of the Bcl-x_{L}mutants ranged from 0.17 to 0.48 Å (Cα atoms).

E92L—In the docking model, the backbone carbonyl of Glu-92 contacts the 033 atom of AA₁, whereas the side chain projects outside the hydrophobic groove with no close ligand contacts. The structure of E92L Bcl-x_{L}(ΔC) at 2.1 Å compared with Bcl-x_{L}^{wt}(ΔC) shows no main chain movement and only a minor displacement between the Leu and Glu side chains. The hydrogen bonds bridging Gln-88 and Asn-198 were missing, weakening interactions between the BH3 α-helical domain and the COOH-terminal region of Bcl-x_{L}(ΔC). Overall, the structure was very similar to wild-type, with a Cα RMSD of 0.17 Å, and an all-atom RMSD of 0.40 Å.

F97W—The Phe-97 residue lies in close proximity to the dilactone ring of AA₁ in the docking model. Thus, the greater bulk of a Trp side chain in this position was expected to cause a steric clash with bound AA₁. The structure of F97W Bcl-x_{L}(ΔC) was solved to 2.7 Å. The F97W substitution did not significantly disrupt the backbone structure (overall RMSD Cα of 0.30 Å, F97W RMSD of 0.29 Å). However, to compensate for the bulkier Trp side chain, Phe-101 rotates about χ₂ approximately 80 degrees. The maximal backbone displacement for F97W Bcl-x_{L}(ΔC) occurred in this region, with residues 101 through 106 having an RMSD Cα of 0.67, although this displacement was significantly less than in the structure of a Bcl-x_{L}/BH3 peptide complex (Protein Data Bank code 1BXL). Thus, the steric effects of Trp-97 on AA₁ affinity should be predominant.

A142L—The A142 residue was positioned adjacent to Phe-97 in the hydrophobic groove. The docking model for AA₁ predicted a significant steric clash between the dilactone ring of AA₁ and the Leu-142 side chain. The structure of A142L Bcl-x_{L}(ΔC) was solved to 2.2 Å. The A142L Bcl-x_{L}(ΔC) structure was very similar to Bcl-x_{L}^{wt}, with a RMSD (Cα) of 0.44 Å. The bulkier leucine side chain caused a compensatory chain of movement of the Phe-97 and Tyr-101 side chains. Furthermore, repositioning of the Tyr-101 backbone promoted alternative orientations of Ala-104 and Phe-105, which flipped from higher energy positive backbone φ/ψ angles to lower energy negative φ/ψ angles. Despite the movement of the Phe-105 main chain, the side chain orientation was conserved with Bcl-x_{L}^{wt}(ΔC). Because Ala-104 and Phe-105 also had negative φ/ψ angles in the BH3 peptide/Bcl-x_{L} structure, the binding pocket in the ligand-bound conformation of A142 Bch x_{L}(ΔC) should be preserved without significant structural changes.

F146L—Unlike the other Bcl-x_{L} mutations considered here, the docking model predicted a loss of van der Waals contacts to the alkyl chain of AA₁ with the F146L substitution. The F146L Bcl-x_{L}(ΔC) structure was solved to 2.2 Å. The F146L Bcl-x_{L}(ΔC) structure showed an overall RMSD (Cα) of 0.49 Å. There was little displacement of the F146L residue compared with Bcl-x_{L}^{wt}(ΔC). The aliphatic and aromatic side chains of the α3 helix and neighboring residues to F146L adopted wild-type orientations with the exception of the rotation of Lys-108 about χ₂. As with the A142L Bcl-x_{L}(ΔC) structure, residues Ala-104 and Phe-105 converted from positive to negative φ/ψ angles. In addition, the Arg-103 backbone had adopted a positive φ/ψ configuration in F146L Bcl-x_{L}. Notably, the average B-factors across all structures, including Bcl-x_{L}^{wt}(ΔC), for residues 101-105 in the α3 helix were about twice that of the rest of the molecule. Thus, the alterations in backbone configuration at residues 103-105 in the Bcl-x_{L}^{mu} proteins may reflect an inherent flexibility of this region. The F146L Bcl-x_{L} structure also demonstrated enlargement of an interior cavity abutted by Phe-146, which may reduce the overall stability of the protein (Baldwin et al., I. Mol. Biol. 259:542-559 (1996); Xu et al., Prot. Sci. 7:158-177 (1998)). In Bcl-x_{L}^{wt}(ΔC), this cavity has a calculated area of 34 Å², which increased to 54 Å² in the F146L mutant structure.

In prior examples set forth above it was demonstrated that expression of the anti-apoptotic protein Bcl-x_{L} rendered cells hypersensitive to antimycin A. Antimycin A bound directly to Bcl-x_{L}(ΔC) in competition with BH3 peptide ligands that occupy the hydrophobic groove, consistent with the identification of this interface as the likely antimycin A binding site by molecular modeling. Site-directed mutagenesis has been used to validate Bcl-x_{L} as a direct target for antimycin A and map the Bcl-x_{L} binding site for antimycin A in greater detail.

Three out of four mutations in the Bcl-x_{L} hydrophobic groove (F97W, A142L, and F146L) eliminated or strongly attenuated the ability of Bcl-x_{L} to sensitize TAMH cells to antimycin A₁ treatment. Each of the mutants had nearly wild-type anti-apoptotic activity with staurosporine treatment, discounting loss of protein function as an explanation for the resistance to antimycin A₁. However, it has been demonstrated that reduced binding affinities of antimycin A₁ with the Bcl-x_{L}^{mu}(ΔC) proteins, with a good correlation between binding constant and in vivo sensitivity to antimycin A₁.

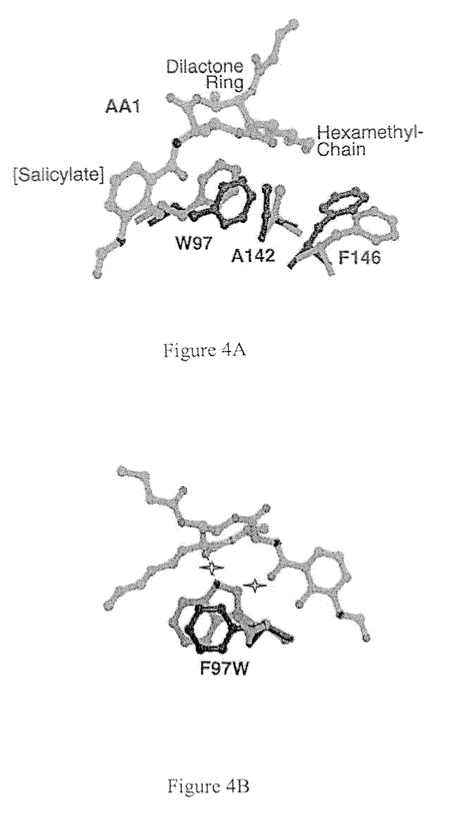

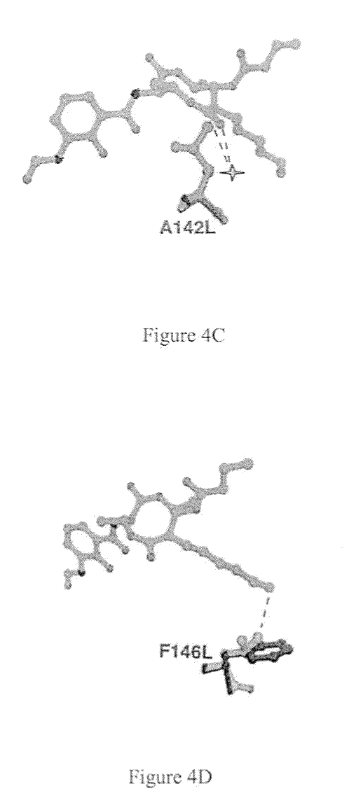

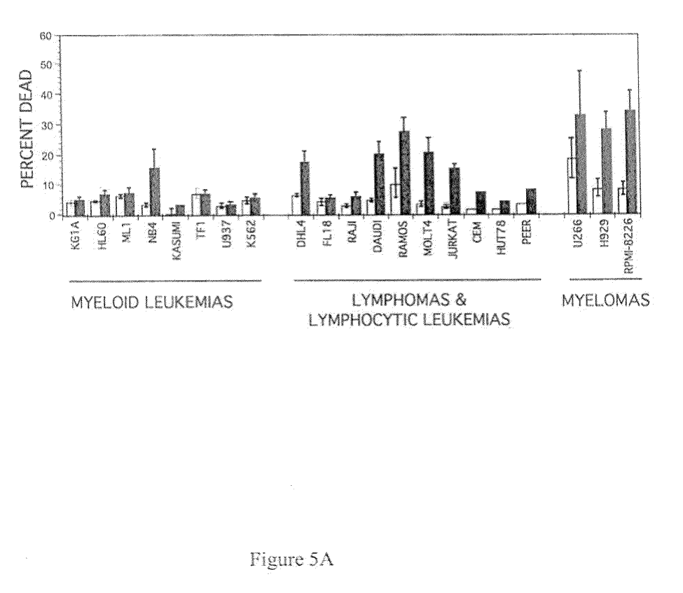

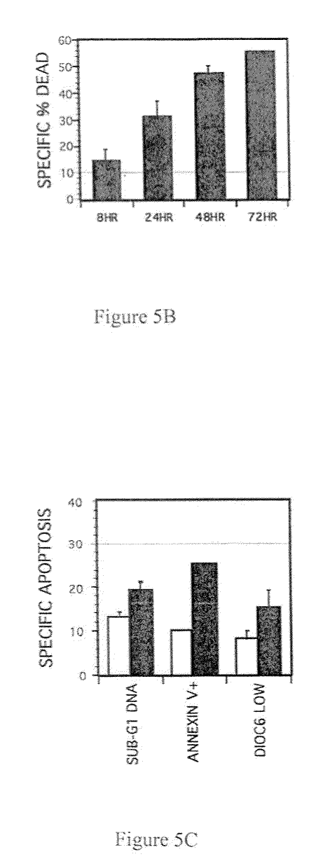

The Bcl-x_{L} mutations were designed for local perturbations on ligand-protein geometry. The crystal structures of the Bcl-x_{L}(ΔC) mutants demonstrated retention of the tertiary protein fold, allowing interpretation of the binding data in terms of the molecular docking model. The docking model for antimycin A₁ utilized the structure of Bcl-x_{L}(ΔC) bound to BAK BH3 peptide. Compared with the overall shift between ligand-bound and free Bcl-x_{L} conformations (Cα RMSD=2.8 Å), there was minimal displacement of the residues predicted to be antimycin A₁ contacts in these structures (Cα RMSD for Phe-97, Ala-142, and Phe-146=1.3 Å) (FIG. 4A).

The relative antimycin A₁ binding affinities of the Bcl-x_{L}^{mu}(ΔC) proteins were as follows: WT>E92L>F97W˜F146L>A142L. Incorporating these single mutations into the AA₁ docking model allowed for the prediction of their effects on AA₁ binding. Both F97W and A142L mutations were modeled to produce steric hindrances to the docked AA₁. In the former case, the increased bulkiness of the tryptophan side chain made close contacts (approximately 2.6 Å) to the dilactone ring of AA₁ (FIG. 4B), whereas the A142L substitution created a significant steric clash to the AA₁ dilactone ring (FIG. 4C). The 8-fold and 20-fold increases in the K_{d} of AA₁ binding for F97W and A142L Bcl-x_{L}^{mu}(ΔC), respectively, compared with Bcl-x_{L}^{wt}(ΔC), were comparable with these predictions. To form a stable complex, significant compensatory movements of the Bcl-x_{L} binding pockets or AA₁ from the starting docking model must occur.

The phenyl group at Phe-146 was oriented perpendicularly to the hexyl-chain of AA₁ in the docking model (FIG. 4D). This predicted interaction consisted of van der Waals contacts and electrostatic interactions between the partial negative charges of the phenyl ring and partial positive charge of the terminal methyl group (C27). Substitution of leucine for phenylalanine removed both types of contacts with AA₁ accounting for the approximate 10-fold decrease in AA₁ affinity with this mutant protein. Overall, the ligand-bound structure models based on the Bcl-x_{L}^{mu}(ΔC) crystal structures provide reasonable interpretations for the measured AA₁ binding constants. The E92L mutation was not expected to alter AA binding based on the docking model, as it was located at the periphery of the binding pocket and contributed only a carbonyl oxygen contact with AA₁. Thus, the 2- to 3-fold reduction of AA₁ binding affinity with the E92L mutation provided an estimate of the general effects on binding for mutations at partially buried residues. The mild reduction in affinity may be due to some destabilization of the Bcl-x_{L}, tertiary structure by the loss of two salt bridges.

The anti-apoptotic activities of Bcl-x_{L} and Bcl-2 act through and are regulated by associations with pro-apoptotic proteins. The Bcl-x_{L}^{mu} proteins retained normal anti-apoptotic activity despite substantially weakened binding to the pro-apoptotic BH3 peptide. Bcl-x_{L} binding affinities for BH3 peptides depended on hydrophobic interactions at the floor of the cleft with several conserved non-polar residues (Val-74, Leu-78, and Ile-81) in the peptides. Modeling studies of the BAK BH3 peptide to the Bcl-x_{L} mutants suggested that the F146L substitution weakened interactions with BAK Val-74, whereas the F97W mutation needed to be moderately displaced to avoid a clash with the side chain of Bak Leu-78. However, Bcl-x_{L} A142L required a much larger adjustment to avoid a clash between the backbone of BAK Leu-78 and the Bcl-x_{L} Leu-142 side chain. Overall, these results suggested that the mutant phenotypes embodied here were more compatible with the pro-apoptotic binding partner exerting negative control over the anti-apoptotic function of Bcl-x_{L}, rather than vice versa. However, only a single pro-apoptotic BH3 domain (BAK) has been tested. For example, Bcl-x_{L} and Bcl-2 proteins with mutations preventing binding to BAK nevertheless strongly interacted with the BH3 only BAD protein in a previous report (Ottillie et al., J. Biol. Chem. 272:272:20866-20872 (1997)).

The hydrophobic groove mutations did not affect the pore-forming ability of purified Bcl-x_{L}(ΔC) in synthetic liposomes, suggesting that this property was endowed by the global protein fold and packing geometry of Bcl-x_{L}(ΔC). AA₁ inhibited pore formation of mutant and wild-type proteins with similar IC₅₀ values. The insensitivity of this assay for discriminating mutants with different AA binding affinities implied AA₁ interacts differently with soluble versus membrane-inserted Bcl-x_{L}(ΔC). Using dansylated lipid in fluorescence resonance energy transfer experiments, it was determined that AA₁ does not interfere with the insertion of Bcl-x_{L} into lipid membranes whereas BAK BH3 inhibits full membrane insertion of Bcl-x_{L}. This finding explained why the distribution of BAK BH3 IC₅₀ values for the Bcl-x_{L}, mutant series reflected the range of affinities for soluble Bcl-x_{L} protein, because pore inhibition took place at a pre-insertion step. The conserved ordering of mutant protein activities in AA binding and pore assays (i.e., WT>E92L>F97W˜F146L>A142L) argued for similarities in the soluble and membrane-inserted binding interfaces. The AA₁-Bcl-x_{L}(ΔC) interaction in a lipid environment appeared to be significantly less constrained by the Bcl-x_{L} mutations considered herein compared with the soluble form of the protein.

The mutational study results in this example strongly support the earlier conclusion set forth above that AA selectively kills Bcl-x_{L} expressing cells by directly targeting Bcl-x_{L}. The single amino acid mutations produced minor shifts in the binding pocket geometry, allowing a high degree of confidence in extrapolating from crystal structures to the ligand-bound protein conformation. The principal basis for the antimycin A resistance of the mutant Bcl-x_{L} proteins appeared to be lower binding affinity, as reflected by the strong correlation between in vitro binding constants and cytotoxicity. In aggregate, these results agreed with the starting molecular model of how AA₁ bound to the Bcl-x_{L} hydrophobic groove.

### Example 12

### In Vitro and In Vivo Effects of 2-Methoxy Antimycin Compounds

High levels of anti-apoptotic Bcl-2 family member proteins are associated with multi-drug resistance in human cancers. (See Reed, Hematol. Oncol. Clin. North Am. 9:451-473 (1995); Amundson et al., Cancer Res. 60:6101-6110 (2000)). In contrast, expression of related pro-apoptotic proteins, such as Bax, increases drug sensitivity in various tumor models. (See McPake et al., Oncol. Res. 10:235-244 (1998)). Heterodimeric interactions between pro- and anti-apoptotic Bcl-2 family members act as an axis for their opposing functions in apoptosis. (See Mahajan et al., Nat. Biotechnol. 16:547-552 (1998)). Pro-apoptotic Bcl-2 homology 3 domain (BH3) peptides that bind to the heterodimer interface of anti-apoptotic Bcl-2 family member proteins initiate apoptotic cell death if introduced into cells. (See Chittenden et al., EMBO J. 14:5589-5596 (1995); Holinger et al., J. Bio. Chem. 274:13298-13304 (1999)). Small molecular mimics of BH3 peptides can be developed as novel anti-cancer drugs, with activity against tumor cells resistant to conventional chemotherapeutic agents. Using a cell-based screen for inhibitors of the anti-apoptotic Bcl-x₁, protein, it was observed that antimycin A, an inhibitor of mitochondrial electron transport at complex III, selectively killed Bcl-x_{L}-expressing cells, as did 2-methoxy analogs (2-OMeAA) without inhibitory action on respiration. (See, Example 10 above, and Tzung et al., Nat. Cell Biol. 3:183-191 (2001)). Specific and stoichiometric binding of antimycin A and 2-OMeAA to recombinant Bcl-x_{L} and Bcl-2 proteins that was efficiently competed by a pro-apoptotic BH3 peptide was subsequently demonstrated. (See Tzung et al., supra; Kim et al., Biochemistry 40:4911-4922 (2001)). It was also determined that 2-OMeAA inhibited the intrinsic pore-forming activity of Bcl-x_{L} in synthetic liposomes, demonstrating that this compound can directly inhibit a molecular function associated with anti-apoptotic Bcl-2 family member proteins. In vivo anti-tumor activity of 2-OMeAA is demonstrated in the following example.

### Experimental Procedures

### Cell Culture

The RPMI-8226 cell line was supplied by W. Dalton (Univ. Arizona). U266 and NCI-H929 cell lines were obtained from the American Type Culture Collection (Rockville, Md.). Cell lines were grown in RPMI 1640 (Gibco, Grand Island, N.Y.) supplemented with 5% fetal bovine serum (Hyclone, Logan, Utah). Normal bone marrow samples were obtained from allogeneic transplant donors at the Fred Hutchinson Cancer Research Center (Seattle, Wash.), with appropriate patient consent and International Review Board approval. Primary cells were maintained in Iscove's medium (Gibco) supplemented with 10% bovine calf serum, 100 ng/ml stem cell factor (Amgen, Thousand Oaks, Calif.), and 50 ng/ml interleukin-3 (Biosource, Camarillo, Calif.).

### GI₅₀ Assays

RPMI-8226 cells were grown in 96 well plates for 24 h prior to addition of 2-methoxy antimycins. Antimycin stocks in dimethyl sulfoxide (DMSO) were serially diluted in RPMI. Cells were incubated for 48 h with compounds at final compound concentrations ranging from 10⁻⁴ to 10⁻⁸ M. MTT cell proliferation assays were performed in quadruplicate and percent growth inhibitions were calculated as (A₅₇₀ (control cells)-A₅₇₀ (treated cells))/A₅₇₀ (control cells). GI₅₀ was extrapolated from semi-log plots of the dose response for each compound. Alternatively, cells were plated in 96-well round-bottomed microplates in complete medium, to which various doses of antimycin A or 2-OMeAA were added 12 to 16 h later, in triplicate. After 72 h drug exposures, 1 μCi/ml [³H]-thymidine was added to wells. Cells were incubated for an additional 24 h, and then transferred to GF/C filter plates (Packard) using a plate washer. Filters were dried, added to scintillant, and counted in a TopCount® scintillation counter (Packard). Drug response is expressed as the percentage of vehicle-treated controls, and each value shown is the average of three determinations.

### Flow Cytometry

Cell survival was assayed by exclusion of propidium iodide (PI) (10 mg/ml) by unfixed cells. Annexin V-FITC (Pharmingen) or 3,3′-dihexyloxacarbocyanine iodide (DiOC₆(3)) (Molecular Probes) staining was analyzed together with PI to discriminate early apoptotic cells. Apoptotic cells were also measured as sub-G₁ events among ethanol-fixed cells stained with 10 mg/ml PI. All cell samples were analyzed using a benchtop FACSCalibur® (Becton Dickinson, San Jose, Calif.) flow cytometer. Flow data were analyzed using MultiCycle AV software (Phoenix Flow Systems). For intracellular pH measurements, cell pellets were washed once and resuspended in 2 ml of HEPES-buffered medium (no phenol red and no serum). Carboxy-SNARF®-1 acetoxymethyl (AM) ester (Molecular Probes, 1 mM stock in DMSO, stored at −20° C.), a long wavelength fluorescent pH indicator, was added to a final concentration of 10 μM, and the cells were incubated for 30 min at 37° C. Following incubation with SNARF®-1 AM, cells were sedimented and the pellets were held on ice. Immediately before analysis, pellets were resuspended in Earle's balanced salt solution (experimental buffer) or high-[K⁺] buffer containing nigericin (calibration samples).

The analysis by flow cytometry (Becton Dickinson FACScan) was done with excitation at 488 nm, and emission at 585 and 640 nm (corresponding to the H⁺-bound and -free forms of carboxy-SNARF®-1 AM, respectively). Determination of the number of cells in the various populations was performed by drawing regions on the profiles generated by analyzing pH calibration samples. The calibration samples were generated by incubating untreated cells with SNARF®-1 AM in high potassium buffers (20 mM NaCl, 130 mM KCl, 1 mM MgCl₂, 1 mM CaCl₂, 5 mM HEPES; titrated to the appropriate pH with HCl and NaOH) containing nigericin (Sigma, 1 mM stock in absolute ethanol, stored at 4° C.) at 10 μM final concentration and fixed pH ranging from 6.5 to 8.5 (nigericin was added after the pH titration).

### Microsequential Injection-Lab on Valve Studies

Detailed methods have been previously published (Schulz et al., Analyst 127:1583-1588 (2002)). Cytopore™ beads (Amersham Pharmacia Biotech, Upsala, Sweden) were hydrated in Hanks balanced salt solution (Gibco-BRL, Grand Island, N.Y.) and autoclaved according to manufacturers instructions. Beads were incubated in serum-containing media overnight. TAMH cells were transferred to the bead slurry at an approximate ratio of 50 cells per bead and grown in spinner culture flasks with gentle stirring. Beads were collected for metabolic studies at cell densities of approximately 100-500 cells/bead. For experiments, cell medium was replaced with HBSS.

Studies were carried out using a FIAlab® 3000 automated sequential injection analyzer (FIAlab Instruments, Medina, Wash.), consisting of a 6-position lab-on-valve (LOV) manifold controlled by a precision bi-directional syringe pump. A 6 port multiposition valve (MPV) with a dedicated syringe pump was added as a bioreactor module. FIAlab software version 5.0 was used to control all of the system components and for data collection and analysis. The flow cell in the LOV was illuminated by a long wave UVA pencil light (Spectronics Corp., Westbury, N.Y.) with a 600 μm UV fiber optic connection to a CCD spectrophotometer (Ocean Optics, Dunedin, Fla.). The entire apparatus was placed inside an incubator set at 37° C.

Glucose and lactate concentrations were assayed as substrates in first-order NAD-linked enzymatic reactions, with NADH generation monitored by absorbance at 340 nm. Infinity™ glucose reagent, glucose and lactate standards, and bovine heart lactate dehydrogenase (LDH) (Sigma, St. Louis, Mo.) were prepared fresh daily. The glucose reagent included final concentrations of >1500 U/L hexokinase, >3200 U/L glucose-6-phosphate dehydrogenase, 2.1 mM ATP and 2.5 mM NAD⁺. The lactate reagent contained final concentrations of 2000 U/ml LDH and 2.5 mM NAD⁺ in glycine buffer.

An assay cycle is initiated by packing a column of cells attached to beads in the microbioreactor, which is upstream of the LOV flow cell. The cells-on-beads were perfused with 2-OMeAA in HBSS, followed by a stop flow period (120 s) to allow depletion of glucose and accumulation of lactate in the interstitial volume of the microbioreactor. After the stop flow period, 3 μL of the interstitial fluid from the cell column is injected through to the LOV flow cell previously loaded with glucose or lactate reagent and 340 nm absorbance recorded for 30 s after mixing. Calibration standards were used to convert endpoint absorbance to concentrations. Each data point represents the mean of three independent measurements done on new columns of TAMH cells-on-beads.

### Mitochondrial Membrane Potential Measurements

RPMI-8226 cells (2×10⁷) were resuspended in buffer containing 130 mM KCl, 5 mM malate, 5 mM glutamate, 2 mM KPO₄, 5 mM HEPES, pH 7.0 with 5 μM safranin O dye, in a stirred cuvette at 28° C. Fluorescence was measured at 495 nm excitation, 586 nm emission on a RF-5301 PC spectrofluorophotometer (Shimadzu, Japan). Safranin 0 fluorescence was quenched following addition of 0.0025% digitonin as the dye accumulated in active mitochondria (Fiskum et al., Methods Enzymol. 322:222-234 (2000)). Depolarization of the mitochondrial inner membrane caused a shift of safranin 0 localization from mitochondria to cytoplasm, evident as increased fluorescence.

### Metabolic Studies of RPMI-8226 Cells

RPMI-8226 cells were starved for glucose by growing in glucose-free RPMI for 24 h in the presence of 2% dialyzed serum and 2 mg/ml sodium pyruvate. This was followed by continuous treatment for 5 and 24 h with AA₁ at 2.5 μM and 2-OMeAA₁ at 1 μM in the presence of indicated concentrations of glucose in RPMI media supplemented with 5% dialyzed serum. The control samples consist of untreated cells, cultured as above and in the presence of 2 mg/ml glucose.

The supernatants from the cells were used for the measurement of glucose concentrations by using a Sigma diagnostic assay kit (Glucose HK assay kit). The manufacturer's procedure was modified, to adapt it to a 96 well plate assay (serial dilution of the glucose standard and samples were made, not to exceed 30-40 μg/well; a maximum of 50 μl of sample was used, in a total volume of 250 μl/well). Data represent micrograms of glucose per 50 μl of supernatant.

### Tumor Xenografts

Six to nine-week old Nod/Les2 Scid/J mice were inoculated with 3×10⁷ RPMI-8226 cells by interscapular subcutaneous injection. Mice were maintained under specific pathogen-free conditions. Palpable tumor nodules were measured in two dimensions with calipers and tumor volumes calculated in mm³ as (length×width²)/2. Blood samples were collected by retro-orbital bleed for human light chain measurements by ELISA with lambda-specific antibody and horseradish peroxidase detection (BD Biosciences). Animals were sacrificed by halothane inhalation, and histologic examination of tumors and internal organs was performed. All experiments were approved by the Fred Hutchinson Cancer Research Center Institutional Animal Care and Use Committee.

### Preparation and In Vivo Dosing of 2-OMe Antimycin A

2-OMe antimycin A₁ was synthesized from antimycin A₁ (Sigma) and dissolved in phosphate-buffered saline with 20% Cremaphor, 25% ethanol (3 mg/ml) for parenteral administration by tail vein injection in a total volume of 100 μl. Control mice received injections of Cremaphor/ethanol vehicle.

### Tissue Sections

Tissues were fixed in 10% buffered formalin, embedded in paraffin, sectioned and stained with hematoxylin and eosin. TUNEL staining was performed using 0.3 units/ml terminal deoxynucleotidyl transferase and biotinylated dATP with development by avidin-biotin peroxidase method. Bcl-x_{L} immunohistochemistry was performed using anti-Bcl-x_{L} antibody (BD Biosciences) followed by biotinylated secondary antibody and peroxidase-labeled ABC reagent (Vector, Burlingame Calif.).

### Results

The cytotoxicity of antimycin A in a panel of human hematopoietic cell lines was assessed using propidium iodide (PI) exclusion assays. Each of the tested myeloma cell lines (RPMI-8226, U266, NCI-H929) was sensitive to 5 to 20 μg/ml antimycin A, as were several leukemia and lymphoma cell lines (DHL4, Daudi, Ramos, Molt4 and Jurkat) (FIG. 5A). RPMI 8226 cell death was demonstrated by 8 h of antimycin A treatment, with significant accumulation of dead cells by 18 to 24 h (FIG. 5B). In contrast, seven of eight myeloid leukemia cell lines were resistant to 20 μg/ml antimycin A, even after 48 h of treatment. NB4, an acute promyelocytic cell line, was modestly antimycin-sensitive.

Antimycin A- and 2-methoxy antimycin A-induced cell death of RPMI-8226 myeloma cells occurred by apoptosis, as demonstrated by accumulations of cells with increased annexin V staining (annexin V⁺, PI⁻), sub-G₁ DNA content, or reduced mitochondrial membrane potential (FIG. 5C and data not shown). Cells treated with AA and 2-OMeAA developed classic apoptotic morphologies as recognized by light microscopy, including fragmented nuclei and marginated chromatin.

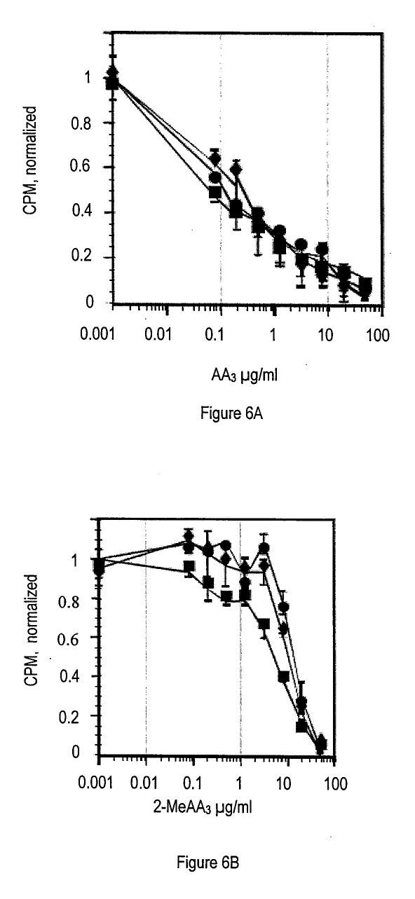

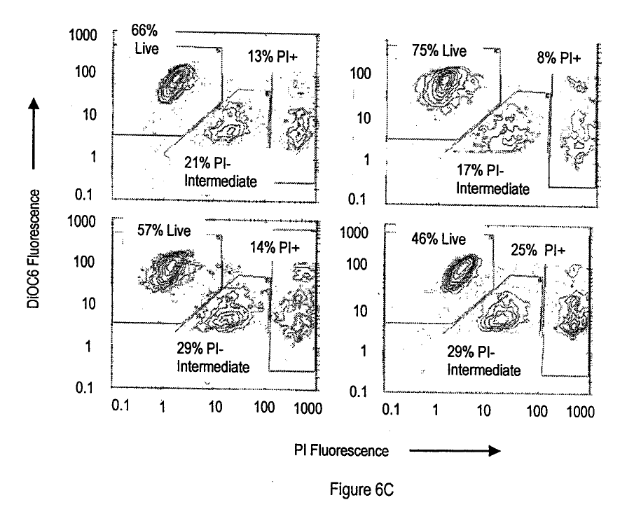

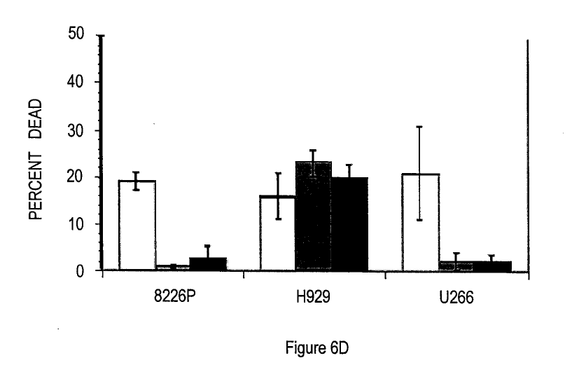

In [³H]-thymidine incorporation assays, AA caused 50% growth inhibition (GI₅₀) of RPMI-8226, U266, and NCI-H929 myeloma cells at 100 ng/ml, 50 ng/ml, and 200 ng/ml, respectively (FIG. 6A). IC₅₀ values for 2-OMeAA-treated cells were 100-fold higher (15, 5, and 10/μg/ml) than with antimycin A, consistent with the negligible inhibition of oxidative phosphorylation by this compound (FIG. 6B) (Tzung et al., Nat. Cell Biol. 3:183-191 (2001); Rieske, Biochim. Biophys Acta. 456:195-247 (1976)). In contrast, cell killing measured as PI uptake, was similar in the three cell lines at antimycin A and 2-OMeAA concentrations of 5 and 20 μg/ml (FIG. 6C).

Oxidative phosphorylation inhibitors at low doses were, in general, not effective at killing myeloma cells. Only H929 cells were killed by oligomycin (2-10 mg/ml), an inhibitor of F0/F1 ATPase, and none of the myeloma cell lines were killed by the complex I inhibitor rotenone (0.5-2.5 mg/ml) (FIG. 6D and data not shown).

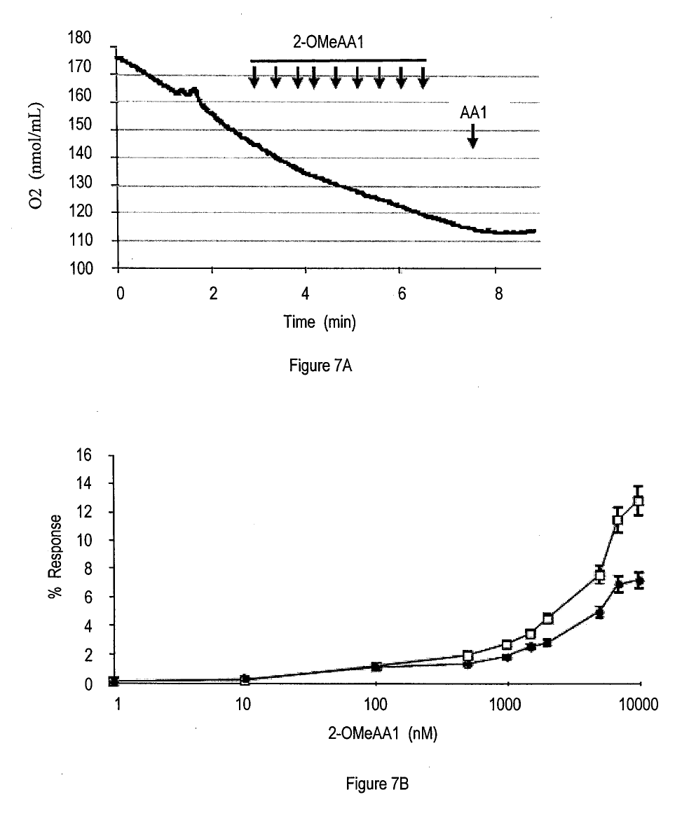

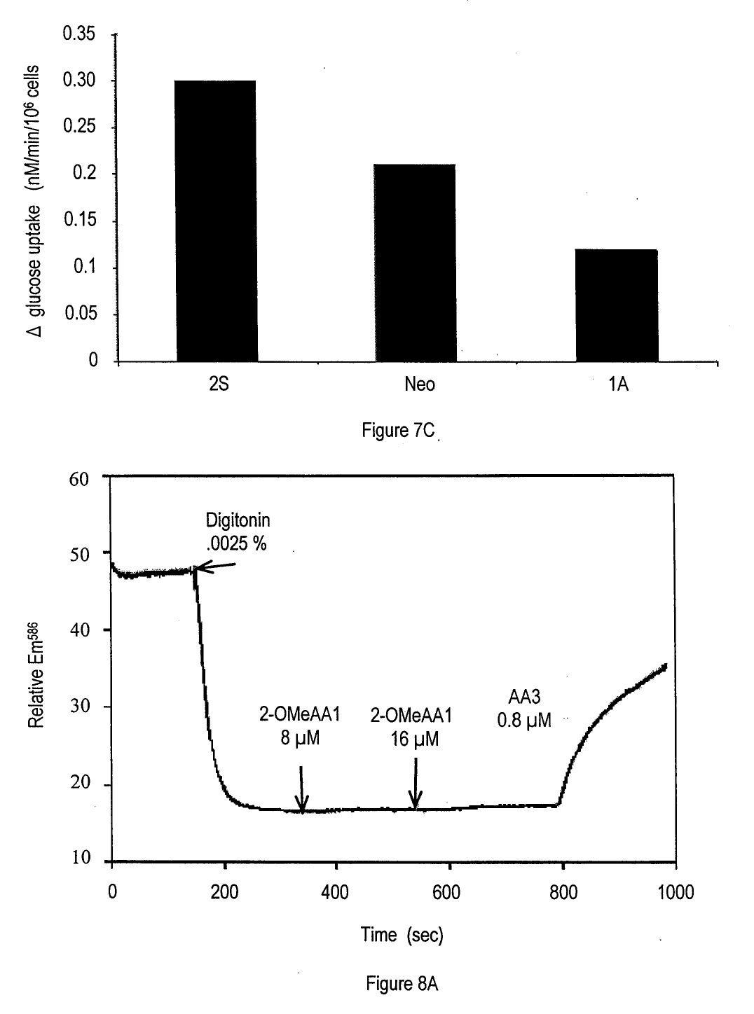

To confirm that the 2-OMe antimycin A did not act to inhibit respiration at the concentrations effective in cytotoxicity assays, O₂ consumption was measured in TAMH hepatocyte and RPMI-8226 myeloma cell lines using a Clark electrode. Respiratory rates of both cell lines were maintained through repeated additions of 10 μM 2-OMeAA, to a final concentration of 90 μM (FIG. 7A). In contrast, addition of 10 μM of antimycin A dampened respiration by >90% and acute decreases in O₂ consumption were observed with 1-2 μM antimycin A. Antimycin A is often used in experimental models of chemical hypoxia. The metabolic response to hypoxia is dominated by a shift to glycolytic metabolism characterized by increased rates of glucose uptake and reductive conversion of pyruvate to lactic acid. Real-time glucose consumption and lactate production by TAMH cells grown on microcarrier beads were monitored using a microsequential injection-lab on valve (μSI-LOV) system (Schulz et al., Analyst 127:1293-1298 (2002)). Antimycin A-treated cells increased glucose uptake and lactate production at doses between 10-100 nM. Unexpectedly, cells treated with 10-100 nM 2-OMeAA demonstrated similar increases in glycolytic metabolism within the first 2 min of treatment (FIG. 7B). Since 2-OMeAA has no effect on cellular respiration at these concentrations, 2-OMeAA induces aerobic glycolysis in TAMH cells. The glycolytic response to 2-OMeAA was proportional to cellular Bcl-x_{L} protein expression (FIG. 7C).

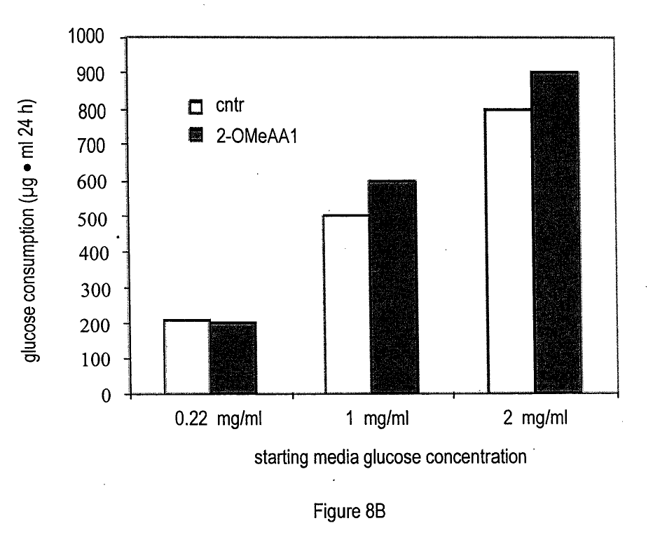

Metabolic responses to 2-OMeAA in RPMI-8226 cells grown in suspension culture were determined. No acute changes in mitochondrial membrane potential were observed with 2-OMeAA, in contrast to the response to low concentrations of antimycin A (FIG. 8A). However, 2-MeOAA treatment stimulated glycolysis in RPMI-8226 cells, assessed as depletion of glucose from the culture media and reduction in intracellular pH (FIG. 8B). Natural antimycin A is composed of several closely related compounds, with the major components represented by antimycin A₁-A₅ (Dickie et al., J. Med. Chem. 6:424-427 (1963)). O-methylated derivatives of antimycin A₁-A₃ and A₅ were tested individually with RPMI-8226 cells (Table 5). Similar growth inhibition (GI₅₀) was observed for 2-OMe antimycin A₁, A₂, and A₃, while 2-OMe antimycin A₅ was approximately ⅓ as active, suggesting that alkyl R groups at positions R₂ and R₁ of the dilactone ring have modest effects on activity. Antimycin A₁ and A₂ share an n-hexyl substituent at position R₁, while A₁ and A₃ have in common an isovaleryl group at position R₂ (Dickie et al., J. Med. Chem. 6:424-427 (1963)). The compound with the highest activity, 2-OMe Antimycin A₁, was selected for in vivo evaluation.

TABLE 5
GI₅₀ concentrations for RPMI-8226 cells treated with 2-methoxy
antimycin compounds.

| COMPOUND | GI₅₀ (μM) |
|---|---|
| 2-OMeAA₁ | 8.56 |
| 2-OMeAA₂ | 12.18 |
| 2-OMeAA₃ | 10.22 |

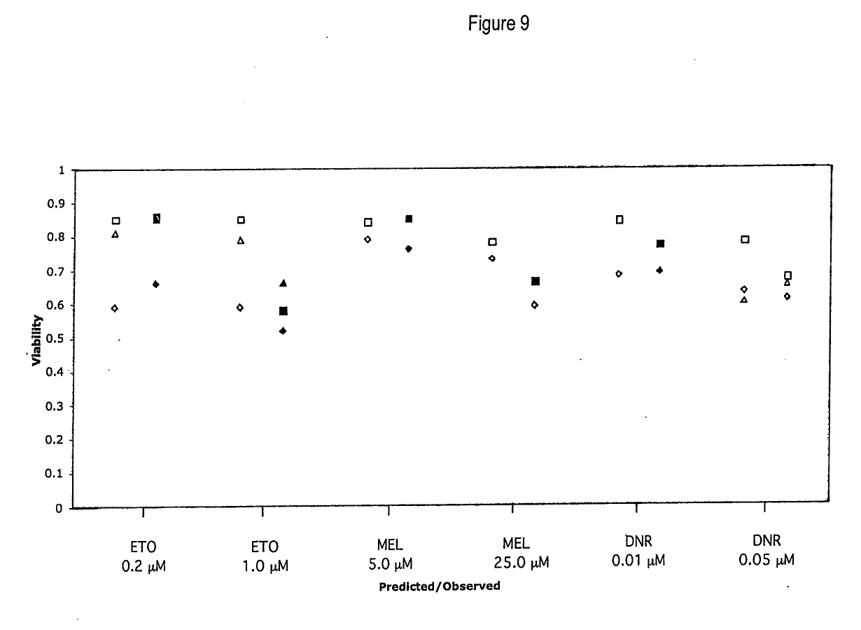

In the TAMH cell lines used to screen for Bcl-x_{L} inhibitors, apoptotic response to 2-OMeAA was inversely related to chemosensitivity with standard agents, consistent with targeting of different cell death pathways. Current anti-myeloma regimens incorporate multiple drugs with different mechanisms of action. RPMI-8226 cells were treated with 2-OMeAA₁ in combination with standard chemotherapeutic agents used in myeloma treatment: etoposide, melphalan, or daunorubicin (Sonneveld and Segeren, Eur. J. Cancer 39:9-18 (2003)). Supra-additive killing was observed with suboptimal combinations of 2-OMeAA₁ and etoposide or melphalan (FIG. 9).

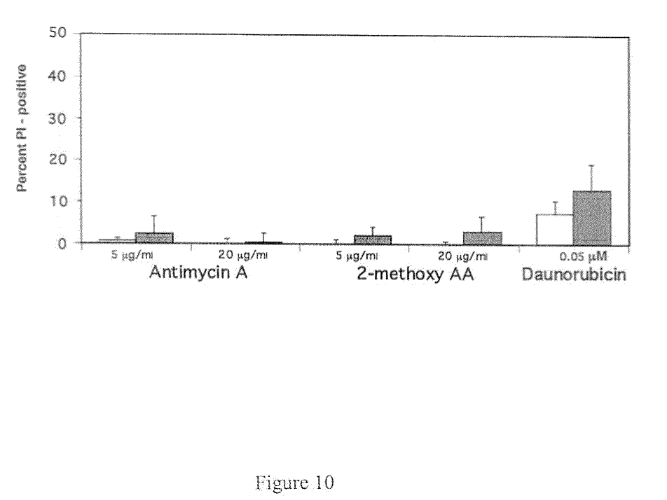

Bcl-x_{L}, is expressed in normal bone marrow hematopoietic precursors, where it is essential for cell survival (Park et al., Blood 86:868-876 (1995); Motoyama et al., Science 267:1506-1510 (1995)). To address the toxicity of 2-OMeAA in normal cells, unfractionated human bone marrow cells were treated in vitro with 2-OMeAA (5-20 μg/ml) for 24-48 h, and cell viability was measured in flow cytometry assays. Primary lymphoid and myeloid bone marrow cell subpopulations, identified in light scatter profiles, were insensitive to 2-OMeAA at these doses as judged by PI (FIG. 10) and annexin V staining (data not shown).

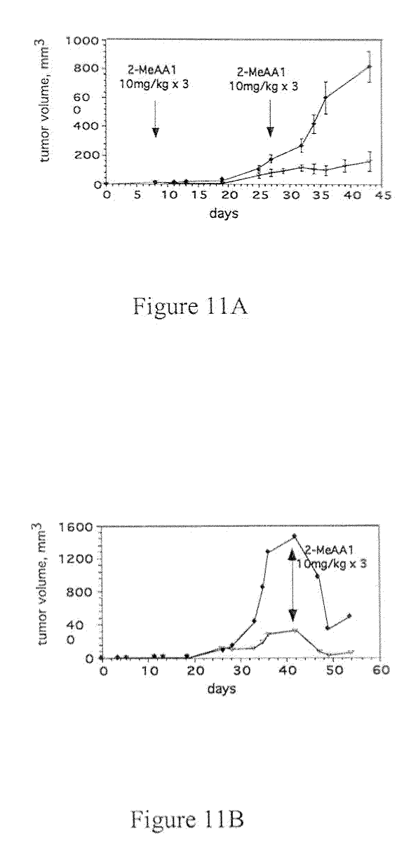

Natural antimycin A is highly lethal in mice with an LD₅₀ of 0.893 mg/kg for a single intravenous dose (Nakayama et al., J. Antibiotics Japan Ser. A. 63-66 (1956)). Although 2-OMeAA does not inhibit mitochondrial respiration at concentrations tested in vitro, its toxicity in vivo was unknown. In particular, the possibility existed that de-methylation could regenerate the highly toxic parent compound in vivo. NOD/SCID mice were treated with three intravenous doses of 2-OMeAA₁ administered at 10 mg/kg on alternate days, and 6/6 mice survived without apparent toxicity. Three of six mice died after intravenous administration of 2-OMeAA at 20 mg/kg. No gross abnormalities were observed at necropsy of treated mice. Therefore, 10 mg/kg dosing was used for testing of in vivo anti-tumor efficacy. To determine whether 2-OMeAA₁ has anti-tumor activity in vivo, a total of 12 NOD/SCID mice in three experiments were inoculated with 3×10⁷ RPMI-8226 human myeloma cells by interscapular subcutaneous injection. In the first experiment, subcutaneous nodules were palpable for seven of eight mice at 4 days after injection, while the remaining mouse had a measurable nodule on day 6, and human lambda light chain was detected by ELISA in serum samples of all mice by day 14. In total, six mice received three intravenous doses of 10 mg/kg 2-OMeAA₁ on alternate days starting on day 6. Six control mice received injections of Cremaphor/ethanol vehicle without drug. One mouse each in the treatment group and the control group died shortly after the third injection. Serum levels of human light chain were reduced an average of 87% in treated mice after the third 2-OMeAA₁ injection. Five of six mice dosed with 2-OMeAA₁ showed tumor regression during treatment, while tumor nodules progressed in all six of the untreated mice (FIG. 11A). At 12 to 15 days after the last dose of 2-OMeAA₁ regrowth of tumor nodules was noted in the treatment group. A second round of alternate day treatments with 10 mg/kg 2-OMeAA₁ on days 26 to 31 again led to regression of tumor nodules in six of six treated mice. A two-way ANOVA of this data reveals a highly significant effect of 2-OMeAA₁ treatment (F=66.83, df=1, p<0.0001).

No adverse effects were noted in any of the mice treated with 2-OMeAA₁. Delayed treatments of two tumor-bearing control mice with 10 mg/kg 2-OMeAA on days 43, 46 and 48 also led to regression of tumor nodules (FIG. 11B).

Tumor sections taken 24 and 48 h after a single treatment with 10 mg/kg 2-OMe AA₁(intravenous) showed widespread apoptosis with numerous fragmented nuclei. Apoptotic nuclei and fragments were also labeled by TUNEL staining. Bcl-x_{L} staining of tumor sections was heterogeneous, similar to the expression of Bcl-2 and Bcl-x_{L} in human solid tumors.

### Discussion

Recent discoveries of several small molecule Bcl-x_{L}, inhibitors with cytotoxic activity have revealed two mechanisms of inhibition, both associated with binding to the hydrophobic groove interface. The compounds BH31-1 and BH31-2 bind to the Bcl-x_{L}, hydrophobic groove with low micromolar affinity and displace pro-apoptotic peptides/proteins (Degterev et al., Nat. Cell Biol. 3:173-182 (2001)). These compounds do not interfere with Bcl-x_{L} membrane pore-forming ability. 2-methoxy antimycin A, a non-toxic analog of the respiratory poison antimycin A, also binds to the Bcl-x_{L} hydrophobic groove with low micromolar affinity, but has weak displacement activity for pro-apoptotic peptides bound at this site (See above and Tzung et al., Nat. Cell. Biol. 3:183-191 (2001); Kim et al., Biochemistry 40:4911-4922 (2001)). In contrast to the BH31 compounds, 2-OMeAA interferes strongly with Bcl-x_{L} pore formation at cytotoxic concentrations.

Methylation of AA at the 2-hydroxyl position of the salicylate ring reduced oxidative phosphorylation inhibitory activity at complex III by 1000-fold. The relative safety of this compound in vivo was evident from its LD₅₀ dose of 20 mg/kg (for a schedule of 3 intravenous doses on alternate days) compared to an LD₅₀ of 0.893 mg/kg (single intravenous dose) for the parent compound (Nakayama et al., J. Antibiotics Japan Ser. A. 63-66 (1956)). The predominant toxicities for antimycin A have been noted in lung, heart and kidney (Greselin and Herr, J. Agric. Food Chem. U. 22:996-998 (1974)). No cellular injury or inflammation was evident in histologic examinations of normal tissues from mice treated with 10 mg/kg 2-OMeAA₁. These results suggest that the parent antimycin A molecule was not regenerated to a significant extent in vivo. In addition, preliminary LC/MS analyses of plasma collected after administration of 2-OMeAA₁ have not demonstrated reformation of antimycin A₁.

Aerobic glycolysis is a hallmark of cancer cells, commonly referred to as the Warburg phenomenon. Warburg postulated that a mitochondrial oxidative phosphorylation defect was a prerequisite for tumorigenesis, with a more gradual up-regulation of glycolysis during progression to neoplasia (Warburg, Science 123:309-314 (1956)). Fixed (intrinsic) deficiencies in oxidative phosphorylation have not been identified as a general feature in cancer, however, despite several decades of investigation. More recently, two transcription factors deregulated in cancers, hypoxia-inducible factor-1 and Myc, have been shown to promote a metabolic shift to aerobic glycolysis by transactivation of glycolytic enzymes and glucose transporters (Semenza et al., Novartis Found Symp. 240:251-260 (2001)). As 2-OMeAA also stimulates aerobic glycolysis without apparently inhibiting oxidative phosphorylation, Bcl-xL may also function as a critical regulator of the balance of oxidative phosphorylation and glycolytic metabolism (Vander Heiden et al., J. Biol. Chem. 277:44870-44876 (2002)).

The 2-OMeAA-sensitive human myeloma cell line RPMI-8226 was xenografted into immunodeficient mice to test the in vivo anti-tumor efficacy of the compound. RPMI-8226 myeloma cells grown as subcutaneous tumor nodules were sensitive to 10 mg/kg 2-OMeAA₁ given intravenously. Regression of both early tumor nodules (<10 mm³) and large nodules (>1000 mm³) was observed within the first week of administering 2-OMeAA₁. Regrowth of early tumor nodules was observed by 10 to 14 days after the initial three doses of 2-OMeAA₁, but a second round of treatment resulted in a more prolonged response.

Normal tissues expressing Bcl-x_{L} include bone marrow, kidney and lymphoid organs (Gonzalez-Garcia et al., Development 120:3033-3042 (1994)). Murine Bcl-x_{L} protein also binds and is inhibited by low micromolar concentrations of 2-OMeAA (See above and Tzung et al., Nat. Cell. Biol. 3:183-191 (2001); Kim et al., Biochemistry 40:4911-4922 (2001)). Nonetheless, there was little evidence of damage to normal tissues in mice treated with 2-OMeAA₁ at doses that caused substantial apoptotic death and macroscopic regression of human myeloma xenografts. The increased susceptibility of tumor cells to 2-OMeAA may be due to the high levels of Bcl-x_{L} or Bcl-2 present in many cancers. As previously demonstrated, the apoptotic response of transfected hepatocyte cell lines to 2-OMeAA was increased with higher cell Bcl-x_{L} levels. This paradoxical effect represents a “gain of function” associated with inhibition of the Bcl-x_{L}-associated pore activity in vitro. Preferential killing of cells with “high” levels of Bcl-x_{L} might afford a desirable therapeutic window for cancer therapy with 2-OMeAA.

The three 2-OMeAA-sensitive human myeloma cell lines (RPMI-8226, U266, NCI-H929) express Bcl-x_{L}, as do the leukemia and lymphoma cell lines that are most sensitive to 2-OMeAA (Tu et al., Cancer Res. 58:256-262 (1998); Catlett-Falcone et al., Immunity 10:105-115 (1999); Yanase et al., J. Interferon Cytokine Res. 18:855-861 (1998); Alam et al., Eur. J. Immunol. 27:3485-3491 (1997); Tagami et al., Oncogene 19:5736-5746 (2000); Campos et al., Leuk Lymphoma 33:499-509 (1999)). Bcl-x_{L}, expression in multiple myeloma has been reported to correlate with disease severity and chemoresistance (Tu et al., Cancer Res. 58:256-262 (1998)). However, Bcl-x_{L} is also expressed prominently in some of the cell lines resistant to 2-OMeAA (e.g., K562). Antimycin A binds to Bcl-2, and may also bind other related anti-apoptotic proteins with conserved hydrophobic clefts (Kim et al., Biochemistry 40:4911-4922 (2001)).

The in vivo response of human myeloma cells to 2-OMeAA demonstrated that endogenous Bcl-2-associated mechanisms of tumor cell survival/drug resistance were viable targets for the treatment of multi-drug resistant cancers and, further, that such pathways can be inhibited without causing significant toxicity. The in vitro findings of improved myeloma cell death when 2-OMeAA was combined with standard myeloma chemotherapeutics further supported the targeting of Bcl-2-associated survival mechanisms for new anti-tumor therapies.

Although the foregoing invention has been described in some detail by way of illustration and example for purposes of clarity of understanding, it will be obvious that certain changes and modifications may be practiced within the scope of the appended claims. The scope of the invention should, therefore, be determined not with reference to the above description, but instead should be determined with reference to the appended claims along with their full scope of equivalents. All publications and patent documents cited in this application are incorporated by reference in their entirety for all purposes to the same extent as if each individual publication or patent document were so individually denoted.

What is claimed is:

1. A method for identifying an agent which modulates apoptosis of a cell by binding to the hydrophobic pocket of an anti-apoptotic Bcl-2 family member protein formed by the BH1, BH2, and BH3 domains of the protein, the method comprising:
(1) admixing a candidate compound independently with each of
(a) a first cell which over-expresses an anti-apoptotic Bcl-2 family member protein; and
(b) a second cell which over-expresses a mutant Bcl-2 family member protein which corresponds to the anti-apoptotic Bcl-2 family member protein, the mutant Bcl-2 family member protein having a mutation in the hydrophobic groove that, relative to the anti-apoptotic Bcl-2 family member protein,
(i) has no substantial effect on tertiary protein structure; and
(ii) reduces binding affinity for an antimycin;
(2) determining whether the candidate compound produces an apoptosis-associated physiological change in the first cell; and
(3) determining whether the candidate compound produces a reduced apoptosis-associated physiological change in the second cell relative to the first cell.

2. The method of claim 1, further comprising the steps of:
(4) admixing the candidate compound with a control cell which does not over-express either the anti-apoptotic or the mutant Bcl-2 family member protein; and
(5) determining whether the candidate compound does not substantially produce the apoptosis-associated physiological change in the control cell.

3. The method of claim 1, wherein the mutation in the hydrophobic groove corresponds to a Bcl-x_{L} mutation is E92L, F97W, L130A, A142L, F146L, or Y195G.

4. The method of claim 1, wherein the anti-apoptotic Bcl-2 family member protein is Bcl-x_{L} or Bcl-2.

5. The method of claim 4, wherein the anti-apoptotic Bcl-2 family member protein is Bcl-x_{L} and the mutation in the hydrophobic groove is E92L, F97W, L130A, A142L, F146L, or Y195G.

6. The method of claim 1, wherein the apoptosis-associated physiological change is cell shrinkage, chromosome condensation and migration, mitochondrial swelling, or disruption of mitochondrial transmembrane potential.

7. The method of claim 6, wherein the cellular change comprises disruption of mitochondrial transmembrane potential.

8. The method of claim 1, wherein the cell that over-expresses the anti-apoptotic Bcl-2 family member protein is transfected with a gene that encodes the anti-apoptotic Bcl-2 family member protein.

9. The method of claim 1, wherein the candidate compound is an antimycin derivative.

10. The method of claim 9, wherein the antimycin derivative is a 2-methoxy antimycin derivative.

11. A method for identifying an agent which modulates apoptosis of a cell by binding to the hydrophobic pocket of an anti-apoptotic Bcl-2 family member protein formed by the BH1, BH-2, and BH3 domains of the protein, the method comprising:
(1) utilizing a molecular docking algorithm to score a candidate compound for binding to the hydrophobic pocket of the Bcl-2 family member protein form by the BH1, BH2, and BH3 domains as determined by protein structure determination for the unliganded protein;
(2) utilizing the molecular docking algorithm to score the candidate compound for binding to the hydrophobic pocket of the Bcl-2 family member protein formed by the BH1, BH2, and BH3 domains as determined by protein structure determination for the protein bound to a BH3 peptide subsequent to subtracting the peptide coordinates; and
(3) determining whether the docking score based on minimal docked conformation energies for (1) is lower than the docking score for (2) to identify the agent.


SEQUENCE LISTING (1 sequence)

SEQ ID NO: 1 - protein, 16 aa, Homo sapiens
GQVGRQLAIIGDDINR